(19) United States
(12) Patent Application Publication
(10) Pub. No.: US 2011/0105491 A1
(43) Pub. Date: May 5, 2011

(54) THIAZOLIDINE COMPOUNDS AS OREXIN RECEPTOR ANTAGONISTS

(76) Inventors: Hamed Aissaoui, Pulversheim (FR); Christoph Boss, Allschwil (CH); Julien Hazemann, Sierentz (FR); Ralf Koberstein, Lorrach (DE); Romain Siegrist, Allschwil (CH); Thierry Sifferlen, Wentzwiller (FR)

(21) Appl. No.: 13/002,365

(22) PCT Filed: Jul. 7, 2009
(86) PCT No.: PCT/IB2009/052949

§ 371 Date: Jan. 3, 2011

(30) Foreign Application Priority Data
Jul. 7, 2008 (IB) PCT/IB2008/052723
Sep. 11, 2008 (IB) PCT/IB2008/053661

(51) Int. Cl. A61K 31/538; C07D 417/14; C07D 513/04; C07D 471/04; C07D 417/10; A61K 31/427; A61K 31/429; A61K 31/437; A61K 31/4245; A61K 31/433; A61K 31/498; A61K 31/4439; A61K 31/4375; A61K 31/497; A61P 25/20; A61P 25/30; A61P 25/00; A61P 25/18; A61P 25/32

(52) U.S. Cl. 514/230.5; 548/200; 548/181; 548/180; 548/154; 546/121; 548/126; 544/105; 544/355; 546/269.7; 546/123; 544/405; 514/365; 514/368; 514/300; 514/364; 514/362; 514/249; 514/342; 514/255.05

(57) ABSTRACT

The invention relates to thiazolidine derivatives of the formula (I) wherein A, B, and R¹ are as described in the description, to salts, especially pharmaceutically acceptable salts, of such compounds and to their use as medicaments, especially as orexin receptor antagonists.

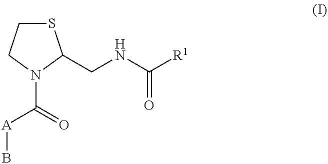

The present invention relates to novel thiazolidine compounds of formula (I) and their use as pharmaceuticals. The invention also concerns related aspects including processes for the preparation of the compounds, pharmaceutical compositions containing one or more compounds of formula (I), and especially their use as orexin receptor antagonists.

Orexins (orexin A or OX-A and orexin B or OX-B) are novel neuropeptides found in 1998 by two research groups, orexin A is a 33 amino acid peptide and orexin B is a 28 amino acid peptide (Sakurai T. et al., Cell, 1998, 92, 573-585). Orexins are produced in discrete neurons of the lateral hypothalamus and bind to G-protein-coupled receptors (OX₁ and OX₂ receptors). The orexin-1 receptor (OX₁) is selective for OX-A, and the orexin-2 receptor (OX₂) is capable to bind OX-A as well as OX-B. Orexins are found to stimulate food consumption in rats suggesting a physiological role for these peptides as mediators in the central feedback mechanism that regulates feeding behaviour (Sakurai T. et al., Cell, 1998, 92, 573-585). On the other hand, it was also observed that orexins regulate states of sleep and wakefulness opening potentially novel therapeutic approaches to narcolepsy as well as insomnia and other sleep disorders (Chemelli R. M. et al., Cell, 1999, 98, 437-451). Furthermore, in vitro and in vivo evidence for a critical role of orexin signaling in the ventral tegmental area in neural plasticity relevant to addiction has been published (S. L. Borgland et al. Neuron, 2006, 49, 589-601).

Thus, orexin receptors may have numerous implications in pathologies as known from the literature, such as dysthymic, mood, psychotic and anxiety disorders; diabetes and appetite, taste, eating, or drinking disorders; hypothalamic diseases; disturbed biological and circadian rhythms; sleep disturbances associated with diseases such as neurological disorders, neuropathic pain and restless leg syndrome; insomnias related to psychiatric disorders; sleep apnea; narcolepsy; idiopathic insomnias; parasomnias; benign prostatic hypertrophy; all dementias and cognitive dysfunctions in the healthy population and in psychiatric and neurologic disorders; and other diseases related to general orexin system dysfunctions. The compound (2R)-2-{(1S)-6,7-dimethoxy-1-[2-(4-trifluoromethyl-phenyl)-ethyl]-3,4-dihydro-1H-isoquinolin-2-yl}-N-methyl-2-phenyl-acetamide (WO2005/118548) is currently in clinical development for primary insomnia. In the rat, the compound has been shown for example to decrease alertness, characterized by decreases in both active wake and locomotion; and to dose-dependently increase the time spent in both REM and NREM sleep (F. Jenck et al., Nature Medicine 2007, 13, 150-155). The compound has also been shown to enhance memory function in a rat model (WO2007/105177) and is also active in a rat model of post-traumatic stress disorder (WO2009/047723).

The present invention provides thiazolidine derivatives, which are non-peptide antagonists of human orexin receptors. These compounds are in particular of potential use in the treatment of e.g. eating disorders, drinking disorders, sleep disorders, or cognitive dysfunctions in psychiatric and neurologic disorders.

Up to now, several low molecular weight compounds are known having a potential to antagonise either specifically OX₁ or OX₂, or both receptors at the same time. Piperidine derivatives useful as orexin receptor antagonists are disclosed in WO01/96302. Morpholine derivatives useful as orexin receptor antagonists are disclosed in WO02/44172. N-Aroyl cyclic amine derivatives useful as orexin receptor antagonists are disclosed in WO02/90355.

The present invention describes novel thiazolidine compounds as orexin receptor antagonists.

i) A first aspect of the invention consists of a compound of the formula (I)

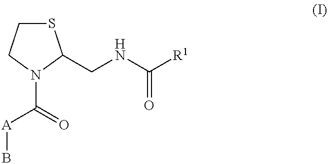

wherein
A represents aryl or heteroaryl, wherein the aryl or heteroaryl is independently unsubstituted or mono- or di-substituted, wherein the substituents are independently selected from the group consisting of (C₁₋₄)alkyl, (C₃₋₆)cycloalkyl, (C₁₋₄)alkoxy, trifluoromethyl, —NR²R³ and halogen;
B represents aryl or heteroaryl, wherein the aryl or heteroaryl is independently unsubstituted or mono-, di-, or tri-substituted, wherein the substituents are independently selected from the group consisting of (C₁₋₄)alkyl, (C₁₋₄)alkoxy, fluoroalkyl, fluoroalkoxy, cyano, and halogen;
R¹ represents aryl or heteroaryl, wherein the aryl or heteroaryl is independently unsubstituted or mono-, di-, or tri-substituted, wherein the substituents are independently selected from the group consisting of (C₁₋₄)alkyl, (C₁₋₄)alkoxy, halogen, cyano, fluoroalkyl, fluoroalkoxy, and —NR²R³; or R¹ represents heterocyclyl wherein said heterocyclyl is unsubstituted or mono- or di-substituted wherein the substituents are independently selected from the group consisting of (C₁₋₄)alkyl, (C₁₋₄)alkoxy, halogen, and oxo;
R² represents hydrogen or (C₁₋₄)alkyl; and
R³ represents hydrogen or (C₁₋₄)alkyl.

In this patent application, a dotted line shows the point of attachment of the radical drawn. For example, the radical drawn below

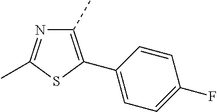

is the 5-(4-fluoro-phenyl)-2-methyl-thiazol-4-yl group.

The term “halogen” means fluorine, chlorine, or bromine, preferably fluorine or chlorine. For the substituent “A” the term halogen preferably means bromine.

The term “alkyl”, used alone or in combination, refers to a straight or branched chain alkyl group containing one to four carbon atoms. The term “(C_{x-y})alkyl” (x and y each being an integer), refers to an alkyl group as defined before containing x to y carbon atoms. For example a (C₁₋₄)alkyl group contains from one to four carbon atoms. Examples of (C₁₋₄)alkyl groups are methyl, ethyl, propyl, isopropyl, n-butyl, isobutyl, sec.-butyl and tert.-butyl. Preferred are methyl and ethyl. Most preferred is methyl.

The term “alkoxy”, used alone or in combination, refers to an alkyl-O— group wherein the alkyl group is as defined before. The term “(C_{x-y})alkoxy” (x and y each being an integer) refers to an alkoxy group as defined before containing x to y carbon atoms. For example a (C₁₋₄)alkoxy group means a group of the formula (C₁₋₄)alkyl-O— in which the term “(C₁₋₄)alkyl” has the previously given significance. Examples of (C₁₋₄)alkoxy groups are methoxy, ethoxy, n-propoxy, isopropoxy, n-butoxy, isobutoxy, sec.-butoxy and tert.-butoxy. Preferred is methoxy.

The term “fluoroalkyl” refers to an alkyl group as defined before containing one to three carbon atoms in which one or more (and possibly all) hydrogen atoms have been replaced with fluorine. The term “(C_{x-y})fluoroalkyl” (x and y each being an integer) refers to a fluoroalkyl group as defined before containing x to y carbon atoms. For example a (C₁₋₃)fluoroalkyl group contains from one to three carbon atoms in which one to seven hydrogen atoms have been replaced with fluorine. Representative examples of fluoroalkyl groups include trifluoromethyl and 2,2,2-trifluoroethyl. Preferred are (C₁)fluoroalkyl groups such as trifluoromethyl.

The term “fluoroalkoxy” refers to an alkoxy group as defined before containing one to three carbon atoms in which one or more (and possibly all) hydrogen atoms have been replaced with fluorine. The term “(C_{x-y})fluoroalkoxy” (x and y each being an integer) refers to a fluoroalkoxy group as defined before containing x to y carbon atoms. For example a (C₁₋₃)fluoroalkoxy group contains from one to three carbon atoms in which one to seven hydrogen atoms have been replaced with fluorine. Representative examples of fluoroalkoxy groups include trifluoromethoxy, difluoromethoxy and 2,2,2-trifluoroethoxy. Preferred are (C₁)fluoroalkoxy groups such as trifluoromethoxy and difluoromethoxy. Most preferred is trifluoromethoxy.

The term “(C₃₋₆)cycloalkyl”, alone or in combination, means a monocyclic saturated alkyl group with 3 to 6 carbon atoms. Examples of (C₃₋₆)cycloalkyl groups are cyclopropyl, cyclobutyl, cyclopentyl, and cyclohexyl. Preferred is cyclopropyl.

The term “—NR²R³” as used for the substituent “A” means for example —NH₂ or notably —N(CH₃)₂.

The term “aryl”, alone or in combination, means a phenyl or a naphthyl group. Preferred is a phenyl group. The aryl group may be unsubstituted or substituted as explicitly defined.

In case “A” represents “aryl” the term means the above-mentioned groups, (preferably phenyl) which are unsubstituted or mono- or di-substituted, wherein the substituents are independently selected from the group consisting of (C₁₋₄)alkyl, (C₃₋₆)cycloalkyl, (C₁₋₄)alkoxy, trifluoromethyl, —NR²R³ and halogen. In a preferred embodiment the term “aryl” as used for the substituent “A” means phenyl which is unsubstituted (preferred) or mono-substituted wherein the substituent is selected from (C₁₋₄)alkyl. An example is phenyl. In addition to the above-mentioned substituents, the substituent “A” is also substituted by the substituent “B”, wherein B is preferably attached in ortho position to the point of attachment of the carbonyl group which links A to the thiazolidine moiety.

In case “B” represents “aryl” the term means the above-mentioned groups, (preferably phenyl) which are unsubstituted or mono-, di-, or tri-substituted, wherein the substituents are independently selected from the group consisting of (C₁₋₄)alkyl, (C₁₋₄)alkoxy, fluoroalkyl, fluoroalkoxy, cyano, and halogen. Preferably the substituents are independently selected from the group consisting of (C₁₋₄)alkyl, (C₁₋₄)alkoxy, fluoroalkyl, fluoroalkoxy, and halogen. In a preferred embodiment the term “aryl” as used for the substituent “B” means phenyl which is unsubstituted or mono-, or di-substituted wherein the substituents are independently selected from the group consisting of (C₁₋₄)alkyl, (C₁₋₄)alkoxy, trifluoromethyl, trifluoromethoxy, and halogen. In another preferred embodiment the term “aryl” as used for the substituent “B” means phenyl which is unsubstituted, or mono-substituted in position 3 or 4 (in a sub-embodiment in position 3, in another sub-embodiment in position 4), or di-substituted wherein the substituents are attached in positions 3 and 4; wherein the substituent(s) are independently selected from the group consisting of methyl, methoxy, trifluoromethyl, trifluoromethoxy, chlorine and fluorine. Examples of aryl groups as used for the substituent “B” are phenyl, 2-methylphenyl, 3-methylphenyl, 4-methylphenyl, 2,3-dimethylphenyl, 2,4-dimethylphenyl, 3,4-dimethylphenyl, 3,5-dimethylphenyl, 4-ethylphenyl, 3-methoxyphenyl, 4-methoxyphenyl, 2-fluorophenyl, 3-fluorophenyl, 4-fluorophenyl, 3,4-difluorophenyl, 3-chlorophenyl, 2,3-dichlorophenyl, 3,4-dichlorophenyl, 3-bromophenyl, 4-bromophenyl, 2-chloro-6-fluorophenyl, 3-bromo-4-fluorophenyl, 3-fluoro-2-methylphenyl, 3-fluoro-4-methylphenyl, 2,3-difluoro-4-methylphenyl, 4-cyanophenyl, 2-trifluoromethylphenyl, 3-trifluoromethylphenyl, 4-trifluoromethylphenyl, 3-fluor-5-trifluoromethylphenyl, and 3-trifluoromethoxyphenyl. Especially, examples are phenyl, 3-methylphenyl, 4-methylphenyl, 3,4-dimethylphenyl, 3-methoxyphenyl, 3-fluorophenyl, 4-fluorophenyl, 3,4-difluorophenyl, 3-chlorophenyl, 3-fluoro-4-methyl phenyl, 2,3-difluoro-4-methylphenyl, 3-trifluoromethylphenyl, 4-trifluoromethylphenyl and 3-trifluoromethoxyphenyl. In addition to the above-mentioned substituents, the substituent “B” is attached to the substituent “A”.

In case “A” and “B” both represents “aryl”, an example of such a combination “A-B” is:

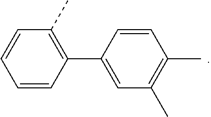

In case “R¹” represents “aryl” the term means the above-mentioned groups which are unsubstituted or mono-, di-, or tri-substituted, wherein the substituents are independently selected from the group consisting of (C₁₋₄)alkyl, (C₁₋₄)alkoxy, halogen, cyano, fluoroalkyl, fluoroalkoxy, and —NR²R³. Preferably the substituents are independently selected from the group consisting of (C₁₋₄)alkyl, (C₁₋₄)alkoxy, fluoroalkyl, fluoroalkoxy, and halogen. In a preferred embodiment the term “aryl” as used for the substituent “R¹” means unsubstituted naphthyl, or phenyl which is unsubstituted or mono-, or di-substituted wherein the substituents are independently selected from the group consisting of (C₁₋₄)alkyl, (C₁₋₄)alkoxy, trifluoromethyl, trifluoromethoxy, and halogen. Examples wherein “R¹” represents “aryl” are 1-naphthyl, 3-methylphenyl, 4-ethylphenyl, 2,3-dimethylphenyl, 2,5-dimethyl-phenyl, 3,4-dimethylphenyl, 3,5-dimethylphenyl, 4-methoxy-2-methylphenyl, 4-methoxy-3-methylphenyl, 2-fluoro-5-methylphenyl, 3-fluoro-2-methylphenyl, 2-chloro-3-methylphenyl, 3-chloro-2-methylphenyl, 2-bromo-5-methylphenyl, 4-methyl-3-trifluoromethylphenyl, 2-methoxyphenyl, 3-methoxyphenyl, 2,3-dimethoxyphenyl, 2,4-dimethoxyphenyl, 2,5-dimethoxyphenyl, 3,4-dimethoxyphenyl, 3,5-dimethoxyphenyl, 3-fluoro-6-methoxyphenyl, 5-fluoro-2-methoxy-phenyl, 3-chloro-6-methoxyphenyl, 4-chloro-2-methoxyphenyl, 5-chloro-2-methoxyphenyl, 4-methoxy-3-trifluoromethylphenyl, 2-chlorophenyl, 3-chlorophenyl, 4-chlorophenyl, 2,4-dichlorophenyl, 3,4-dichlorophenyl, 3-bromophenyl, 4-bromophenyl, 3-iodophenyl, 2-chloro-3-fluorophenyl, 2-chloro-4-fluorophenyl, 5-bromo-2-chlorophenyl, 2-chloro-4,5-difluorophenyl, 3-cyanophenyl, 4-cyanophenyl, 3,5-bis(trifluoromethyl)phenyl and 3-trifluoromethylphenyl.

The term “heteroaryl”, alone or in combination, means a 5- to 10-membered monocyclic or bicyclic aromatic ring containing 1, 2 or 3 heteroatoms independently selected from oxygen, nitrogen and sulfur. Examples of such heteroaryl groups are furanyl, oxazolyl, isoxazolyl, oxadiazolyl, thiophenyl, thiazolyl, isothiazolyl, thiadiazolyl, pyrrolyl, imidazolyl, pyrazolyl, triazolyl, pyridyl, pyrimidyl, pyridazinyl, pyrazinyl, indolyl, isoindolyl, benzofuranyl, isobenzofuranyl, benzothiophenyl, indazolyl, benzimidazolyl, benzoxazolyl, benzisoxazolyl, benzothiazolyl, benzoisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl and imidazo[2,1-b]thiazolyl. The heteroaryl group may be unsubstituted or substituted as explicitly defined.

In case “A” represents “heteroaryl” the term means the above-mentioned groups. In another embodiment, in case “A” represents “heteroaryl” the term means a 5- to 6-membered (especially 5-membered) monocyclic heteroaryl as defined above. In another embodiment, in case “A” represents “heteroaryl” the term means a 5- to 6-membered (especially 5-membered) monocyclic heteroaryl selected from thiophenyl, oxazolyl, thiazolyl, pyrazolyl, pyrimidyl, pyrazinyl, and pyridyl. In another embodiment, in case “A” represents “heteroaryl” said heteroaryl is selected from the group consisting of thiophenyl (notably thiophen-2-yl and especially thiophen-3-yl), oxazolyl (notably oxazol-4-yl), and thiazolyl (notably thiazol-5-yl and especially thiazol-4-yl); and in addition to the above-mentioned groups pyrazinyl (notably pyrazin-2-yl); wherein each of the above-mentioned groups constitute a particular sub-embodiment. The above-mentioned heteroaryl groups as used for the substituent “A” are unsubstituted or mono- or di-substituted, wherein the substituents are independently selected from the group consisting of (C₁₋₄)alkyl, (C₃₋₆)cycloalkyl, (C₁₋₄)alkoxy, trifluoromethyl, —NR²R³ and halogen. In another embodiment, the above-mentioned heteroaryl groups as used for the substituent “A” are unsubstituted or mono-substituted, wherein the substituents are independently selected from the group consisting of (C₁₋₄)alkyl, (C₃₋₆)cycloalkyl, (C₁₋₄)alkoxy, trifluoromethyl, —NR²R³ and halogen. In another embodiment, the above-mentioned heteroaryl groups as used for the substituent “A” are unsubstituted or mono-substituted, wherein the substituent is selected from the group consisting of (C₁₋₄)alkyl, (C₃₋₆)cycloalkyl, and —NR²R³. In another embodiment, particular groups as used for the substituent “A” are preferably substituted as follows: pyrazinyl groups are preferably unsubstituted; thiophenyl groups are preferably unsubstituted; oxazolyl groups are preferably mono-substituted, wherein the substituent is selected from (C₁₋₄)alkyl (especially methyl); thiazol-5-yl groups are preferably mono-substituted, wherein the substituent is selected from (C₁₋₄)alkyl (especially methyl); thiazol-4-yl groups as used for the substituent “A” are preferably unsubstituted or mono-substituted, wherein the substituent is selected from the group consisting of (C₁₋₄)alkyl (especially methyl), (C₃₋₆)cycloalkyl (especially cyclopropyl), (C₁₋₄)alkoxy (especially methoxy), trifluoromethyl, —NR²R³ (especially —NH₂ or —N(CH₃)₂) and halogen (especially bromo); especially, thiazol-4-yl groups as used for the substituent “A” are unsubstituted or mono-substituted, wherein the substituent is selected from the group consisting of (C₁₋₄)alkyl (notably methyl), (C₃₋₆)cycloalkyl (notably cyclopropyl), and —NR²R³ (notably —N(CH₃)₂). Particular examples wherein “A” represents “heteroaryl” are thiophen-2-yl, thiophen-3-yl, 2-methyl-oxazol-4-yl, 2-methyl-thiazol-5-yl, thiazol-4-yl, 2-methyl-thiazol-4-yl, 2-amino-thiazol-4-yl, 2-dimethylamino-thiazol-4-yl, 2-bromo-thiazol-4-yl, 2-methoxy-thiazol-4-yl and 2-cyclopropyl-thiazol-4-yl; and in addition to the above-mentioned groups pyrazin-2-yl. In a sub-embodiment, particular examples of said groups are thiophen-3-yl, 2-methyl-oxazol-4-yl, 2-methyl-thiazol-5-yl, thiazol-4-yl, 2-methyl-thiazol-4-yl, 2-dimethylamino-thiazol-4-yl, and 2-cyclopropyl-thiazol-4-yl. In another sub-embodiment particular examples are 2-methyl-thiazol-4-yl, 2-dimethylamino-thiazol-4-yl, and 2-cyclopropyl-thiazole-4-yl; wherein each group forms a particular sub-embodiment. In another sub-embodiment a particular example is 2-methyl-thiazol-5-yl. In another sub-embodiment a particular example is thiophen-3-yl. In yet another sub-embodiment a particular example is 2-methyl-oxazol-4-yl. In yet another sub-embodiment a particular example is pyrazin-2-yl.

In addition to the above-mentioned substituents, the substituent “A” is also substituted by the substituent “B”, wherein “B” is preferably attached in ortho position to the point of attachment of the carbonyl group which links A to the thiazolidine moiety. In particular, in the above-mentioned examples of heteroaryl groups as used for the substituent “A”, the substituent “B” is preferably attached as follows: in position 5 of thiazol-4-yl groups, in position 4 of thiazol-5-yl groups, in position 3 of thiophen-2-yl groups, in position 2 of thiophen-3-yl groups, in position 5 of oxazol-4-yl groups, and in position 3 of pyrazin-2-yl groups.

In case “A” represents “heteroaryl”, and “B” represents “aryl”, examples of such a combination “A-B” are selected from:

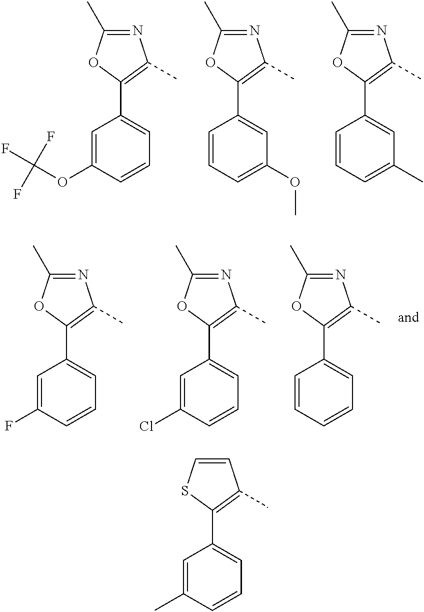

In addition to the above-listed examples, in case “A” represents “heteroaryl”, and “B” represents “aryl”, further examples of such a combination “A-B” are selected from:

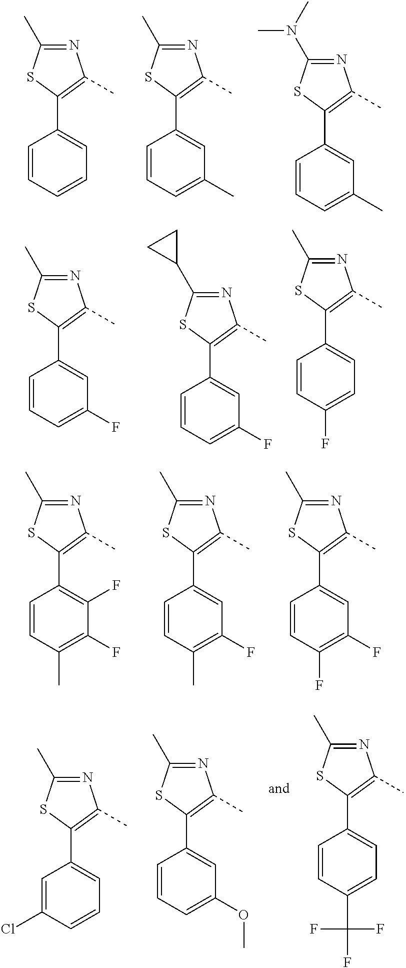

In addition to the above-listed examples, in case “A” represents “heteroaryl”, and “B” represents “aryl”, further examples of such a combination “A-B” are selected from:

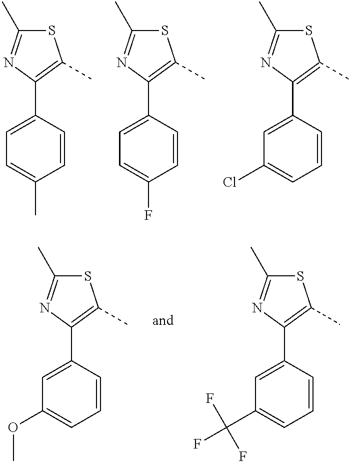

In addition to the above-listed examples, in case “A” represents “heteroaryl”, and “B” represents “aryl”, further examples of such a combination “A-B” are selected from:

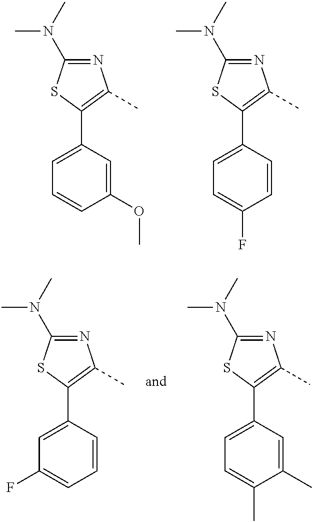

In addition to the above-listed examples, in case “A” represents “heteroaryl”, and “B” represents “aryl”, further examples of such a combination “A-B” are selected from:

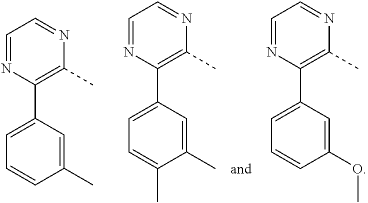

In case “B” represents “heteroaryl” the term means the above-mentioned groups. In a preferred embodiment, in case “B” represents “heteroaryl” the term means a 5- to 6-membered monocyclic heteroaryl as defined above. In another preferred embodiment, in case “B” represents “heteroaryl” the term means a 5- to 6-membered monocyclic heteroaryl selected from thiophenyl, oxazolyl, thiazolyl, pyrazolyl, pyrimidyl, pyrazinyl, and pyridyl. The above-mentioned heteroaryl groups as used for the substituent “B” are unsubstituted or mono-, di-, or tri-substituted, wherein the substituents are independently selected from the group consisting of (C₁₋₄)alkyl, (C₁₋₄)alkoxy, fluoroalkyl, fluoroalkoxy, cyano, and halogen. Preferably the substituents are independently selected from the group consisting of (C₁₋₄)alkyl, (C₁₋₄)alkoxy, fluoroalkyl, fluoroalkoxy, and halogen. In a preferred embodiment, the above-mentioned heteroaryl groups as used for the substituent “B” are unsubstituted or mono-, or di-substituted wherein the substituents are independently selected from the group consisting of (C₁₋₄)alkyl, (C₁₋₄)alkoxy, trifluoromethyl, and halogen (especially methyl, methoxy, trifluoromethyl, chlorine and fluorine).

In case “R¹” represents “heteroaryl” the term means the above-mentioned groups. In a preferred embodiment, in case “R¹” represents “heteroaryl” the term means a group selected from the group consisting of furanyl, oxazolyl (notably oxazol-5-yl, oxazol-4-yl), isoxazolyl (notably isoxazol-3-yl, isoxazol-4-yl), oxadiazolyl, thiophenyl, thiazolyl (notably thiazol-2-yl, thiazol-4-yl, thiazol-5-yl), isothiazolyl, thiadiazolyl, pyrrolyl, imidazolyl, pyrazolyl (notably pyrazol-3-yl, pyrazol-4-yl, pyrazol-5-yl), triazolyl, pyridyl (notably pyridin-2-yl, pyridin-3-yl, pyridin-4-yl), pyrimidyl (notably pyrimidin-2-yl, pyrimidin-4-yl, pyrimidin-5-yl), pyridazinyl, pyrazinyl, indolyl (notably indol-2-yl, indol-3-yl, indol-4-yl, indol-7-yl), isoindolyl, benzofuranyl (notably benzofuran-4-yl, benzofuran-7-yl), isobenzofuranyl, benzothiophenyl (notably benzothiophene-3-yl, benzothiophene-4-yl, benzothiophene-7-yl), indazolyl (notably 1H-indazol-3-yl, 1H-indazol-4-yl, 1H-indazol-7-yl), benzimidazolyl (notably 1H-benzimidazol-4-yl, 1H-benzoimidazol-5-yl, 3H-benzoimidazol-5-yl, 1H-benzimidazol-7-yl), benzoxazolyl (notably benzoxazol-4-yl, benzoxazol-7-yl), benzisoxazolyl (notably benzisoxazol-3-yl, benzisoxazol-4-yl, benzisoxazol-7-yl), benzothiazolyl (notably benzothiazol-4-yl, benzothiazol-7-yl), benzoisothiazolyl (notably benzoisothiazol-3-yl, benzoisothiazol-4-yl, benzoisothiazol-7-yl), benzotriazolyl (notably benzotriazol-5-yl), benzo[2,1,3]oxadiazolyl (notably benzo[2,1,3]oxadiazol-4-yl), benzo[2,1,3]thiadiazolyl (notably benzo[2,1,3]thiadiazol-4-yl), benzo[1,2,3]thiadiazolyl (notably benzo[1,2,3]thiadiazol-5-yl), quinolinyl (notably quinolin-2-yl, quinolin-8-yl), isoquinolinyl (notably isoquinolin-1-yl), naphthyridinyl (notably [2,6]naphthyridin-3-yl, [1,5]naphthyridin-2-yl, [1,8]naphthyridin-2-yl), cinnolinyl, quinazolinyl, quinoxalinyl (notably quinoxalin-5-yl, quinoxalin-2-yl), phthalazinyl, pyrazolo[1,5-a]pyridyl (notably pyrazolo[1,5-a]pyridin-3-yl), pyrazolo[1,5-a]pyrimidyl, imidazo[1,2-a]pyridyl (notably imidazo[1,2-a]pyridin-3-yl), 1H-pyrrolo[3,2-b]pyridyl (notably 1H-pyrrolo[3,2-b]pyridin-4-yl), 1H-pyrrolo[2,3-b]pyridyl (notably 1H-pyrrolo[2,3-b]pyridin-4-yl, 1H-pyrrolo[2,3-b]pyridin-5-yl), 4H-furo[3,2-b]pyrrolyl (notably 4H-furo[3,2-b]pyrrol-5-yl), pyrrolo[2,1-b]thiazolyl (notably pyrrolo[2,1-b]thiazol-7-yl) and imidazo[2,1-b]thiazolyl (notably imidazo[2,1-b]thiazol-2-yl, imidazo[2,1-b]thiazol-3-yl, imidazo[2,1-b]thiazol-5-yl, imidazo[2,1-b]thiazol-6-yl), and in addition to the above-mentioned groups also 1H-pyrazolo[3,4-b]pyridyl (notably 1H-pyrazolo[3,4-b]pyridin-5-yl), and 1H-pyrazolo[3,2-b]pyridyl (notably 1H-pyrazolo[3,2-b]pyridin-6-yl). In a sub-embodiment, in case “R¹” represents “heteroaryl” the term means a group selected from isoxazolyl, pyrazolyl, pyridyl, pyrimidyl, indolyl, benzofuranyl, benzothiophenyl, indazolyl, benzimidazolyl, benzoxazolyl, benzisoxazolyl, benzothiazolyl, benzoisothiazolyl benzotriazolyl, benzo[2,1,3]oxadiazolyl, benzo[2,1,3]thiadiazolyl, benzo[1,2,3]thiadiazolyl, quinolinyl, isoquinolinyl, quinoxalinyl, pyrazolo[1,5-a]pyridyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl, imidazo[2,1-b]thiazolyl, and in addition to the above-mentioned groups also oxazolyl, and thiazolyl; wherein the specific points of attachment of said groups are preferably as mentioned above. In another sub-embodiment, in case “R¹” represents “heteroaryl” the term means a group selected from isoxazolyl, pyrazolyl, indolyl, benzofuranyl, indazolyl, benzimidazolyl, benzoxazolyl, benzisoxazolyl, benzothiazolyl, benzoisothiazolyl, benzotriazolyl, benzo[2,1,3]oxadiazolyl, benzo[2,1,3]thiadiazolyl, benzo[1,2,3]thiadiazolyl, quinoxalinyl, imidazo[1,2-a]pyridyl, pyrrolo[2,1-b]thiazolyl, imidazo[2,1-b]thiazolyl, and in addition to the above-mentioned groups also pyridyl, oxazolyl, and thiazolyl; wherein the specific points of attachment of said groups are preferably as mentioned above. In another sub-embodiment, in case “R¹” represents “heteroaryl” the term means a group selected from benzofuranyl, indazolyl, benzoxazolyl, benzothiazolyl, benzoisothiazolyl benzo[2,1,3]thiadiazolyl, imidazo[1,2-a]pyridyl, and pyrrolo[2,1-b]thiazolyl, and in addition to the above-mentioned groups also pyridyl, oxazolyl, and thiazolyl; wherein the specific points of attachment of said groups are preferably as mentioned above.

The above-mentioned heteroaryl groups as used for the substituent “R¹” are unsubstituted or mono- or di-substituted (especially unsubstituted or mono-substituted) wherein the substituents are independently selected from the group consisting of (C₁₋₄)alkyl, (C₁₋₄)alkoxy, trifluoromethyl, —NR²R³ and halogen. In a preferred embodiment, the above-mentioned heteroaryl groups as used for the substituent “R¹” are unsubstituted or mono- or di-substituted (especially unsubstituted or mono-substituted) wherein the substituents are independently selected from the group consisting of (C₁₋₄)alkyl, (C₁₋₄)alkoxy, trifluoromethyl, and halogen (especially (C₁₋₄)alkyl, trifluoromethyl, and halogen). In another preferred embodiment, the above-mentioned heteroaryl groups as used for the substituent “R¹” are unsubstituted or mono- or di-substituted (especially unsubstituted or mono-substituted), wherein the substituents are independently selected from the group consisting of (C₁₋₄)alkyl and halogen (especially (C₁₋₄)alkyl). In particular, the above mentioned “heteroaryl” groups as used for the substituent “R¹” are preferably substituted as follows: oxazolyl groups are unsubstituted, mono-, or di-substituted (preferred) wherein the substituents are independently selected from (C₁₋₄)alkyl (especially methyl); isoxazolyl groups are mono-, or di-substituted (preferred) independently with (C₁₋₄)alkyl; thiazolyl groups are unsubstituted, mono-, or di-substituted (preferred mono-, or di-substituted) wherein the substituents are independently selected from (C₁₋₄)alkyl (especially methyl); pyrazolyl groups are mono-, di-, or tri-substituted wherein the substituents are independently selected from (C₁₋₄)alkyl, trifluoromethyl and halogen; pyridyl groups are mono-, or di-substituted (preferred mono-substituted) wherein the substituents are independently selected from (C₁₋₄)alkyl (especially methyl), (C₁₋₄)alkoxy (especially methoxy), trifluoromethyl and halogen (especially chloro); benzofuranyl groups are unsubstituted, or mono-, or di-substituted wherein the substituents are independently selected from (C₁₋₄)alkyl (especially methyl), trifluoromethyl and halogen (especially benzofuranyl groups are mono-, or di-substituted wherein the substituents are independently selected from (C₁₋₄)alkyl (notably methyl) and halogen); indolyl groups are unsubstituted, or mono-, or di-substituted wherein the substituents are independently selected from (C₁₋₄)alkyl (especially methyl) and halogen; especially indolyl groups are mono-substituted with (C₁₋₄)alkyl (notably methyl); indazolyl groups are unsubstituted, or mono-substituted with (C₁₋₄)alkyl (especially methyl); benzimidazolyl groups are unsubstituted or mono-substituted wherein the substituent is (C₁₋₄)alkyl; benzotriazolyl groups are mono-substituted with (C₁₋₄)alkyl (especially methyl); benzoxazolyl groups are unsubstituted (preferred), or mono-substituted with (C₁₋₄)alkyl (especially methyl); benzothiazolyl groups are unsubstituted (preferred), or mono-substituted with (C₁₋₄)alkyl (especially methyl) or halogen (especially chlorine); quinolinyl groups are unsubstituted (preferred), or mono-substituted with (C₁₋₄)alkyl or (C₁₋₄)alkoxy; quinoxalinyl groups are unsubstituted (preferred), or mono-substituted with (C₁₋₄)alkyl; benzisoxazolyl, benzisothiazolyl, benzo[2,1,3]thiadiazolyl, benzo[1,2,3]thiadiazolyl, benzo[2,1,3]oxadiazolyl, isoquinolinyl, naphthyridinyl, pyrazolo[3,4-b]pyridinyl, pyrazolo[3,2-b]pyridinyl, and pyrazolo[1,5-a]pyridinyl groups are unsubstituted; 4H-furo[3,2-b]pyrrolyl groups are mono-substituted with (C₁₋₄)alkyl (especially methyl); pyrrolo[2,1-b]thiazolyl groups are unsubstituted (preferred), or mono-substituted with (C₁₋₄)alkyl (especially methyl); imidazo[1,2-a]pyridinyl groups are unsubstituted (preferred), or mono- or di-substituted independently with (C₁₋₄)alkyl; imidazo[2,1-b]thiazolyl groups are unsubstituted, or mono-, or di-substituted wherein the substituents are independently selected from (C₁₋₄)alkyl (especially methyl), trifluoromethyl and halogen (especially imidazo[1,2-a]pyridinyl groups are unsubstituted, or mono-substituted with (C₁₋₄)alkyl (notably methyl)).

Particular examples of “R¹” representing “heteroaryl” are 2,5-dimethyl-2H-pyrazol-3-yl, 2-ethyl-5-methyl-2H-pyrazol-3-yl, 1-isopropyl-1H-pyrazol-4-yl, 1,3-dimethyl-1H-pyrazol-4-yl, 1-ethyl-3-methyl-1H-pyrazol-4-yl, 1-methyl-5-trifluoromethyl-1H-pyrazol-4-yl, 1,3,5-trimethyl-1H-pyrazol-4-yl, 5-chloro-1,3-dimethyl-1H-pyrazol-4-yl, 3,5-dimethyl-isoxazol-4-yl, 5-ethyl-3-methyl-isoxazol-4-yl, 3-ethyl-5-methyl-isoxazol-4-yl, 5-fluoro-1-methyl-1H-indol-2-yl, 1H-indol-3-yl, 1,2-dimethyl-1H-indol-3-yl, 1-methyl-1H-indol-3-yl, 1-methyl-1H-indol-4-yl, 1-methyl-1H-indol-5-yl, 1-methyl-1H-indol-7-yl, benzofuran-4-yl, 2-methyl-benzofuran-4-yl, 3-methyl-benzofuran-4-yl, 2,3-dimethyl-benzofuran-4-yl, 5-chloro-2-methyl-benzofuran-4-yl, 6-chloro-2-methyl-benzofuran-4-yl, 7-chloro-2-methyl-benzofuran-4-yl, 6-fluoro-2-methyl-benzofuran-4-yl, 7-fluoro-2-methyl-benzofuran-4-yl, 6-trifluoromethyl-2-methyl-benzofuran-4-yl, 7-trifluoromethyl-2-methyl-benzofuran-4-yl, benzofuran-7-yl, 1H-benzimidazol-4-yl, 3H-benzoimidazol-5-yl, 1H-indazol-3-yl, 1H-indazol-4-yl, 1H-indazol-7-yl, 1-methyl-1H-indazol-3-yl, benzoxazol-4-yl, 2-methyl-benzoxazol-4-yl, benzoxazol-7-yl, 2-methyl-benzoxazol-7-yl, benzo[d]isoxazol-3-yl, benzothiazol-7-yl, benzothiazol-6-yl, benzothiazol-4-yl, 2-chloro-benzothiazol-4-yl, 2-methyl-benzothiazol-4-yl, benzoisothiazol-3-yl, 1-methyl-benzotriazol-5-yl, benzo[2,1,3]oxadiazol-4-yl, benzo[2,1,3]thiadiazol-4-yl, benzo[1,2,3]thiadiazol-5-yl, quinolin-8-yl, isoquinolin-1-yl, quinoxalin-5-yl, pyrrolo[2,1-b]thiazol-7-yl, 6-methyl-pyrrolo[2,1-b]thiazol-7-yl, pyrazolo[1,5-a]pyridin-3-yl, imidazo[1,2-a]pyridin-3-yl, 2-methyl-imidazo[1,2-a]pyridin-3-yl, 2,8-dimethyl-imidazo[1,2-a]pyridin-3-yl, 1H-pyrrolo[3,2-b]pyridin-4-yl, 1H-pyrrolo[2,3-b]pyridin-4-yl, 1H-pyrrolo[2,3-b]pyridin-5-yl, 4H-furo[3,2-b]pyrrol-5-yl, imidazo[2,1-b]thiazol-5-yl, 6-methyl-imidazo[2,1-b]thiazol-5-yl, 2-methyl-imidazo[2,1-b]thiazol-5-yl, 2,6-dimethyl-imidazo[2,1-b]thiazol-5-yl, 3-methyl-imidazo[2,1-b]thiazol-5-yl, 3,6-dimethyl-imidazo[2,1-b]thiazol-5-yl, 2,3,6-trimethyl-imidazo[2,1-b]thiazol-5-yl, 6-chloro-imidazo[2,1-b]thiazol-5-yl, 6-trifluoromethyl-imidazo[2,1-b]thiazol-5-yl, imidazo[2,1-b]thiazol-6-yl, 5-methyl-imidazo[2,1-b]thiazol-6-yl, 3,5-dimethyl-imidazo[2,1-b]thiazol-6-yl, and 3-methyl-imidazo[2,1-b]thiazol-2-yl. In addition to the above-mentioned groups further particular examples are 4-chloro-pyridine-2-yl, 4-chloro-pyridine-3-yl, 5-chloro-pyridine-2-yl, 4-methoxy-pyridine-2-yl, 2-methoxy-pyridine-3-yl, 6-methyl-pyridine-2-yl, 6-methoxy-pyridine-2-yl, 2,6-dimethoxy-pyridine-3-yl, 6-methyl-pyridine-3-yl, 5-methyl-pyridine-3-yl, 4-methyl-pyridine-3-yl, 6-trifluoromethyl-pyridine-2-yl, 4-trifluoromethyl-pyridine-3-yl, 6-trifluoromethyl-pyridine-3-yl, 2,5-dimethyl-oxazol-4-yl, 2,4-dimethyl-oxazol-5-yl, 4-methyl-oxazol-5-yl, oxazol-4-yl, 2,4-dimethyl-thiazol-5-yl, 2-methyl-thiazol-4-yl, 4-methyl-thiazol-5-yl, 4-methyl-thiazol-2-yl, 5-methyl-thiazol-2-yl, thiazol-4-yl, 5-methyl-isoxazol-3-yl, 1H-pyrazolo[3,4-b]pyridin-5-yl, 1H-pyrazolo[3,2-b]pyridin-6-yl, quinoxalin-2-yl, 3-methyl-quinoxalin-2-yl, [2,6]naphthyridin-3-yl, [1,5]naphthyridin-2-yl, and [1,8]naphthyridin-2-yl. In a sub-embodiment, examples of “R¹” representing “heteroaryl” are 1-isopropyl-1H-pyrazol-4-yl, 1,3-dimethyl-1H-pyrazol-4-yl, 1-ethyl-3-methyl-1H-pyrazol-4-yl, 1-methyl-5-trifluoromethyl-1H-pyrazol-4-yl, 1,3,5-trimethyl-1H-pyrazol-4-yl, 5-chloro-1,3-dimethyl-1H-pyrazol-4-yl, 3,5-dimethyl-isoxazol-4-yl, 5-ethyl-3-methyl-isoxazol-4-yl, 3-ethyl-5-methyl-isoxazol-4-yl, 1H-indol-3-yl, 1-methyl-1H-indol-3-yl, 1-methyl-1H-indol-4-yl, 1-methyl-1H-indol-5-yl, 1-methyl-1H-indol-7-yl, benzofuran-4-yl, 2-methyl-benzofuran-4-yl, 3-methyl-benzofuran-4-yl, 2,3-dimethyl-benzofuran-4-yl, 5-chloro-2-methyl-benzofuran-4-yl, 6-chloro-2-methyl-benzofuran-4-yl, 7-chloro-2-methyl-benzofuran-4-yl, 6-fluoro-2-methyl-benzofuran-4-yl, 7-fluoro-2-methyl-benzofuran-4-yl, 6-trifluoromethyl-2-methyl-benzofuran-4-yl, 7-trifluoromethyl-2-methyl-benzofuran-4-yl, benzofuran-7-yl, 1H-benzimidazol-4-yl, 3H-benzoimidazol-5-yl, 1H-indazol-3-yl, 1H-indazol-4-yl, 1H-indazol-7-yl, 1-methyl-1H-indazol-3-yl, benzoxazol-4-yl, 2-methyl-benzoxazol-4-yl, benzoxazol-7-yl, 2-methyl-benzoxazol-7-yl, benzo[d]isoxazol-3-yl, benzothiazol-6-yl, benzothiazol-7-yl, benzoisothiazol-3-yl, benzo[2,1,3]oxadiazol-4-yl, benzo[2,1,3]thiadiazol-4-yl, benzo[1,2,3]thiadiazol-5-yl, pyrrolo[2,1-b]thiazol-7-yl, 6-methyl-pyrrolo[2,1-b]thiazol-7-yl, imidazo[1,2-a]pyridin-3-yl, 2-methyl-imidazo[1,2-a]pyridin-3-yl, imidazo[2,1-b]thiazol-5-yl, and 6-methyl-imidazo[2,1-b]thiazol-5-yl, and in addition to the above-mentioned groups also 4-chloro-pyridine-2-yl, 4-methoxy-pyridine-2-yl, 2-methoxy-pyridine-3-yl, 6-methyl-pyridine-2-yl, 6-methoxy-pyridine-2-yl, 6-trifluoromethyl-pyridine-2-yl, 2,5-dimethyl-oxazol-4-yl, 2,4-dimethyl-oxazol-5-yl, 2,4-dimethyl-thiazol-5-yl, 2-methyl-thiazol-4-yl, 4-methyl-thiazol-5-yl, 4-methyl-thiazol-2-yl, and 5-methyl-thiazol-2-yl. In another sub-embodiment, examples of “R¹” representing “heteroaryl” are 1-ethyl-3-methyl-1H-pyrazol-4-yl, 1-methyl-5-trifluoromethyl-1H-pyrazol-4-yl, 3,5-dimethyl-isoxazol-4-yl, 5-ethyl-3-methyl-isoxazol-4-yl, 3-ethyl-5-methyl-isoxazol-4-yl, 1-methyl-1H-indol-3-yl, 1-methyl-1H-indol-4-yl, 1-methyl-1H-indol-7-yl, 2-methyl-benzofuran-4-yl, 3-methyl-benzofuran-4-yl, 2,3-dimethyl-benzofuran-4-yl, 5-chloro-2-methyl-benzofuran-4-yl, 6-chloro-2-methyl-benzofuran-4-yl, 7-chloro-2-methyl-benzofuran-4-yl, 6-fluoro-2-methyl-benzofuran-4-yl, 7-fluoro-2-methyl-benzofuran-4-yl, 6-trifluoromethyl-2-methyl-benzofuran-4-yl, 7-trifluoromethyl-2-methyl-benzofuran-4-yl, 1H-indazol-4-yl, 1-methyl-1H-indazol-3-yl, benzoxazol-4-yl, 2-methyl-benzoxazol-4-yl, benzoxazol-7-yl, 2-methyl-benzoxazol-7-yl, benzo[d]isoxazol-3-yl, benzothiazol-6-yl, benzothiazol-7-yl, benzoisothiazol-3-yl, benzo[2,1,3]oxadiazol-4-yl, benzo[2,1,3]thiadiazol-4-yl, benzo[1,2,3]thiadiazol-5-yl, pyrrolo[2,1-b]thiazol-7-yl, 6-methyl-pyrrolo[2,1-b]thiazol-7-yl, imidazo[1,2-a]pyridin-3-yl, 2-methyl-imidazo[1,2-a]pyridin-3-yl, imidazo[2,1-b]thiazol-5-yl, and 6-methyl-imidazo[2,1-b]thiazol-5-yl, and in addition to the above-mentioned groups also 4-chloro-pyridine-2-yl, 4-methoxy-pyridine-2-yl, 2-methoxy-pyridine-3-yl, 6-methyl-pyridine-2-yl, 6-methoxy-pyridine-2-yl, 6-trifluoromethyl-pyridine-2-yl, 2,5-dimethyl-oxazol-4-yl, 2,4-dimethyl-oxazol-5-yl, 2,4-dimethyl-thiazol-5-yl, 2-methyl-thiazol-4-yl, 4-methyl-thiazol-5-yl, 4-methyl-thiazol-2-yl, and 5-methyl-thiazol-2-yl. In another sub-embodiment, examples of “R¹” representing “heteroaryl” are 2-methyl-benzofuran-4-yl, 3-methyl-benzofuran-4-yl, 2,3-dimethyl-benzofuran-4-yl, 6-fluoro-2-methyl-benzofuran-4-yl, 1-methyl-1H-indazol-3-yl, benzoxazol-4-yl, 2-methyl-benzoxazol-7-yl, benzothiazol-7-yl, benzoisothiazol-3-yl, benzo[2,1,3]thiadiazol-4-yl, pyrrolo[2,1-b]thiazol-7-yl, and imidazo[1,2-a]pyridin-3-yl, and in addition to the above-mentioned groups also 4-chloro-pyridine-2-yl, 4-methoxy-pyridine-2-yl, 2-methoxy-pyridine-3-yl, 6-methyl-pyridine-2-yl, 6-methoxy-pyridine-2-yl, 6-trifluoromethyl-pyridine-2-yl, 2,5-dimethyl-oxazol-4-yl, 2,4-dimethyl-oxazol-5-yl, 2,4-dimethyl-thiazol-5-yl, 2-methyl-thiazol-4-yl, 4-methyl-thiazol-5-yl, 4-methyl-thiazol-2-yl, and 5-methyl-thiazol-2-yl.

The term “heterocyclyl”, alone or in combination, means a phenyl ring fused to a 5- or 6-membered saturated or unsaturated non-aromatic ring containing 1 or 2 heteroatoms independently selected from the group consisting of oxygen and nitrogen. Examples of heterocyclyl groups as used for the substituent R¹ are 2,3-dihydro-benzofuranyl (especially 2,3-dihydro-benzofuran-4-yl or 2,3-dihydro-benzofuran-7-yl), 4H-benzo[1,3]dioxinyl (especially 4H-benzo[1,3]dioxin-8-yl or 4H-benzo[1,3]dioxin-5-yl), benzo[1,3]dioxolyl (especially benzo[1,3]dioxol-4-yl), 3,4-dihydro-2H-benzo[1,4]oxazinyl (especially 3,4-dihydro-2H-benzo[1,4]oxazin-5-yl or 3,4-dihydro-2H-benzo[1,4]oxazin-8-yl), 2,3-dihydro-benzo[1,4]dioxinyl (especially 2,3-dihydro-benzo[1,4]dioxin-5-yl, 2,3-dihydro-benzo[1,4]dioxin-6-yl), 2H-chromenyl (especially 2H-chromen-5-yl), and chromanyl (especially chroman-5-yl or chroman-8-yl). The above-mentioned heterocyclyl groups are unsubstituted, or mono-, or di-substituted wherein the substituents are independently selected from (C₁₋₄)alkyl, (C₁₋₄)alkoxy, halogen and oxo (especially from (C₁₋₄)alkyl, halogen and oxo; notably from (C₁₋₄)alkyl and oxo). Preferably, the above-mentioned heterocyclyl groups are substituted as follows: 2,3-dihydro-benzofuranyl-groups are unsubstituted or independently di-substituted with (C₁₋₄)alkyl, (C₁₋₄)alkoxy and halogen (especially unsubstituted, or di-substituted in position 2 with methyl); 4H-benzo[1,3]dioxinyl-groups are preferably unsubstituted, or mono-substituted in position 6 with fluoro; benzo[1,3]dioxolyl-groups are preferably unsubstituted, or di-substituted in position 2 with fluoro; 3,4-dihydro-2H-benzo[1,4]oxazinyl-groups are preferably unsubstituted, or mono- or di-substituted with (C₁₋₄)alkyl (especially methyl) or oxo; wherein, in a sub-embodiment, a (C₁₋₄)alkyl substituent is preferably attached to the nitrogen atom and an oxo substituent is preferably attached in alpha-position of the nitrogen atom of the benzo[1,4]oxazinyl group; 2,3-dihydro-benzo[1,4]dioxinyl-, 2H-chromenyl-, and chromanyl- groups are preferably unsubstituted. Particular examples of such heterocyclyl groups are 3,4-dihydro-2H-benzo[1,4]oxazin-5-yl, 3,4-dihydro-2H-benzo[1,4]oxazin-8-yl, 3-oxo-3,4-dihydro-2H-benzo[1,4]oxazin-5-yl, 3-oxo-3,4-dihydro-2H-benzo[1,4]oxazin-8-yl, 4-methyl-3,4-dihydro-2H-benzo[1,4]oxazin-5-yl, 4-methyl-3,4-dihydro-2H-benzo[1,4]oxazin-8-yl, 4-methyl-3-oxo-3,4-dihydro-2H-benzo[1,4]oxazin-5-yl, 4-methyl-3-oxo-3,4-dihydro-2H-benzo[1,4]oxazin-8-yl, 2,3-dihydro-benzofuran-4-yl, 2,3-dihydro-benzofuran-7-yl, 2,2-dimethyl-2,3-dihydro-benzofuran-7-yl, 4H-benzo[1,3]dioxin-5-yl, 6-fluoro-4H-benzo[1,3]dioxin-8-yl, 4H-benzo[1,3]dioxin-8-yl, benzo[1,3]dioxol-4-yl, 2,2-difluoro-benzo[1,3]dioxol-4-yl, 2,3-dihydro-benzo[1,4]dioxin-5-yl, 2,3-dihydro-benzo[1,4]dioxin-6-yl, 2H-chromen-5-yl, chroman-5-yl and chroman-8-yl. In a sub-embodiment, particular examples are 3,4-dihydro-2H-benzo[1,4]oxazin-5-yl, 3,4-dihydro-2H-benzo[1,4]oxazin-8-yl, 3-oxo-3,4-dihydro-2H-benzo[1,4]oxazin-8-yl, 4-methyl-3,4-dihydro-2H-benzo[1,4]oxazin-5-yl, 4-methyl-3,4-dihydro-2H-benzo[1,4]oxazin-8-yl, 4-methyl-3-oxo-3,4-dihydro-2H-benzo[1,4]oxazin-8-yl, 2,3-dihydro-benzofuran-4-yl, 6-fluoro-4H-benzo[1,3]dioxin-8-yl, 2,3-dihydro-benzo[1,4]dioxin-5-yl, 2,3-dihydro-benzo[1,4]dioxin-6-yl, chroman-5-yl and chroman-8-yl. In another sub-embodiment, particular examples are 2,3-dihydro-benzofuran-4-yl and 2,3-dihydro-benzo[1,4]dioxin-5-yl.

Further embodiments of the invention are presented hereafter:

2) A further embodiment of the invention relates to compounds of formula (I) according to embodiment 1), which are also compounds of formula (I_{E1}) wherein the stereocenter at position 2 of the thiazolidine moiety is in absolute (S)-configuration:

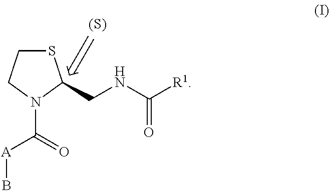

3) A further embodiment of the invention relates to compounds of formula (I) according to embodiment 1), which are also compounds of formula (I_{E2}) wherein the stereocenter at position 2 of the thiazolidine moiety is in absolute (R)-configuration:

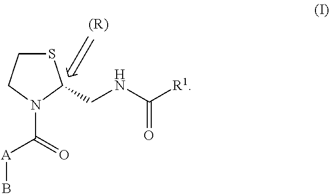

4) A further embodiment of the invention relates to compounds of formula (I) according to any one of embodiments 1) to 3), wherein A represents aryl or heteroaryl, wherein the aryl or heteroaryl is independently unsubstituted or mono-substituted, wherein the substituents are independently selected from the group consisting of (C₁₋₄)alkyl, (C₃₋₆)cycloalkyl, and —NR²R³.
5) A further embodiment of the invention relates to compounds of formula (I) according to any one of embodiments 1) to 4), wherein A represents phenyl which is unsubstituted (preferred) or mono-substituted wherein the substituent is selected from (C₁₋₄)alkyl.
6) A further embodiment of the invention relates to compounds of formula (I) according to any one of embodiments 1) to 4), wherein A represents a 5- to 6-membered (especially 5-membered) monocyclic heteroaryl which is unsubstituted or mono-substituted, wherein the substituent is selected from the group consisting of (C₁₋₄)alkyl, (C₃₋₆)cycloalkyl, and —NR²R³.
7) A further embodiment of the invention relates to compounds of formula (I) according to any one of embodiments 1) to 4), wherein A represents heteroaryl selected from the group consisting of thiophenyl (notably thiophen-2-yl and especially thiophen-3-yl), oxazolyl (notably oxazol-4-yl), thiazolyl (notably thiazol-5-yl and especially thiazol-4-yl), and pyrazinyl (notably pyrazin-2-yl); wherein said heteroaryl are unsubstituted or mono-substituted, wherein the substituent is selected from the group consisting of (C₁₋₄)alkyl, (C₃₋₆)cycloalkyl, and —NR²R³.
8) A further embodiment of the invention relates to compounds of formula (I) according to any one of embodiments 1) to 4), wherein A represents a group selected from the group consisting of thiophen-2-yl, thiophen-3-yl, 2-methyl-oxazol-4-yl, 2-methyl-thiazol-5-yl, thiazol-4-yl, 2-methyl-thiazol-4-yl, 2-amino-thiazol-4-yl, 2-dimethylamino-thiazol-4-yl, 2-bromo-thiazol-4-yl, 2-methoxy-thiazol-4-yl, 2-cyclopropyl-thiazol-4-yl, and pyrazin-2-yl (in a sub-embodiment A represents a group selected from the group consisting of thiophen-2-yl, thiophen-3-yl, 2-methyl-oxazol-4-yl, 2-methyl-thiazol-5-yl, thiazol-4-yl, 2-methyl-thiazol-4-yl, 2-amino-thiazol-4-yl, 2-dimethylamino-thiazol-4-yl, 2-bromo-thiazol-4-yl, 2-methoxy-thiazol-4-yl, 2-cyclopropyl-thiazol-4-yl).
9) A further embodiment of the invention relates to compounds of formula (I) according to any one of embodiments 1) to 4), wherein A represents a group selected from the group consisting of 2-methyl-thiazol-4-yl, 2-dimethylamino-thiazol-4-yl, 2-cyclopropyl-thiazole-4-yl, and pyrazin-2-yl (in a sub-embodiment A represents a group selected from the group consisting of 2-methyl-thiazol-4-yl, 2-dimethylamino-thiazol-4-yl, and 2-cyclopropyl-thiazole-4-yl).
10) A further embodiment of the invention relates to compounds of formula (I) according to any one of embodiments 1) to 9), wherein B represents aryl or heteroaryl, wherein the aryl or heteroaryl is independently unsubstituted or mono-, di-, or tri-substituted, wherein the substituents are independently selected from the group consisting of (C₁₋₄)alkyl, (C₁₋₄)alkoxy, fluoroalkyl, fluoroalkoxy, and halogen.
11) A further embodiment of the invention relates to compounds of formula (I) according to any one of embodiments 1) to 10), wherein B represents aryl which is unsubstituted or mono-, di-, or tri-substituted, wherein the substituents are independently selected from the group consisting of (C₁₋₄)alkyl, (C₁₋₄)alkoxy, fluoroalkyl, fluoroalkoxy, and halogen.
12) A further embodiment of the invention relates to compounds of formula (I) according to any one of embodiments 1) to 10), wherein B represents phenyl which is unsubstituted, or mono-substituted in position 3 or 4 (in a sub-embodiment in position 3, in another sub-embodiment in position 4), or di-substituted wherein the substituents are attached in positions 3 and 4; wherein the substituent(s) are independently selected from the group consisting of methyl, methoxy, trifluoromethyl, trifluoromethoxy, chlorine and fluorine.
13) A further embodiment of the invention relates to compounds of formula (I) according to any one of embodiments 1) to 12), wherein R¹ represents heteroaryl, which is unsubstituted or mono- or di-substituted (especially unsubstituted or mono-substituted) wherein the substituents are independently selected from the group consisting of (C₁₋₄)alkyl, (C₁₋₄)alkoxy, trifluoromethyl, and halogen (especially (C₁₋₄)alkyl, trifluoromethyl, and halogen); or R¹ represents heterocyclyl wherein said heterocyclyl is unsubstituted or mono- or di-substituted wherein the substituents are independently selected from the group consisting of (C₁₋₄)alkyl, halogen and oxo (especially (C₁₋₄)alkyl and oxo).
14) A further embodiment of the invention relates to compounds of formula (I) according to any one of embodiments 1) to 13), wherein R¹ represents heteroaryl, which is unsubstituted or mono- or di-substituted (especially unsubstituted or mono-substituted) wherein the substituents are independently selected from the group consisting of (C₁₋₄)alkyl, (C₁₋₄)alkoxy, trifluoromethyl, and halogen (especially (C₁₋₄)alkyl, trifluoromethyl, and halogen).
15) A further embodiment of the invention relates to compounds of formula (I) according to any one of embodiments 1) to 13), wherein R¹ represents heterocyclyl wherein said heterocyclyl is unsubstituted or mono- or di-substituted wherein the substituents are independently selected from the group consisting of (C₁₋₄)alkyl, halogen and oxo (especially (C₁₋₄)alkyl and oxo);
16) A further embodiment of the invention relates to compounds of formula (I) according to any one of embodiments 1) to 14), wherein, in case R¹ represents heteroaryl, said heteroaryl is selected from the group consisting of isoxazolyl, pyrazolyl, pyridyl, pyrimidyl, indolyl, benzofuranyl, benzothiophenyl, indazolyl, benzimidazolyl, benzoxazolyl, benzisoxazolyl, benzothiazolyl, benzoisothiazolyl benzotriazolyl, benzo[2,1,3]oxadiazolyl, benzo[2,1,3]thiadiazolyl, benzo[1,2,3]thiadiazolyl, quinolinyl, isoquinolinyl, quinoxalinyl, pyrazolo[1,5-a]pyridyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl, imidazo[2,1-b]thiazolyl, oxazolyl, and thiazolyl (in a sub-embodiment, in case R¹ represents heteroaryl, said heteroaryl is selected from the group consisting of isoxazolyl, pyrazolyl, pyridyl, pyrimidyl, indolyl, benzofuranyl, benzothiophenyl, indazolyl, benzimidazolyl, benzoxazolyl, benzisoxazolyl, benzothiazolyl, benzoisothiazolyl benzotriazolyl, benzo[2,1,3]oxadiazolyl, benzo[2,1,3]thiadiazolyl, benzo[1,2,3]thiadiazolyl, quinolinyl, isoquinolinyl, quinoxalinyl, pyrazolo[1,5-a]pyridyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl and imidazo[2,1-b]thiazolyl); wherein said heteroaryl is unsubstituted or mono- or di-substituted (especially unsubstituted or mono-substituted) wherein the substituents are independently selected from the group consisting of (C₁₋₄)alkyl, (C₁₋₄)alkoxy, trifluoromethyl, and halogen (especially (C₁₋₄)alkyl, trifluoromethyl, and halogen).
17) A further embodiment of the invention relates to compounds of formula (I) according to any one of embodiments 1) to 14), wherein, in case R¹ represents heterocyclyl, said heterocyclyl is selected from the group consisting of 2,3-dihydro-benzofuranyl, 4H-benzo[1,3]dioxinyl, benzo[1,3]dioxolyl, 3,4-dihydro-2H-benzo[1,4]oxazinyl, 2,3-dihydro-benzo[1,4]dioxinyl, 2H-chromenyl, and chromanyl, wherein said heterocyclyl is unsubstituted or mono- or di-substituted wherein the substituents are independently selected from the group consisting of (C₁₋₄)alkyl, halogen and oxo (especially (C₁₋₄)alkyl and oxo).
18) A further embodiment of the invention relates to compounds of formula (I) according to any one of embodiments 1) to 14), wherein, in case R¹ represents heteroaryl, said heteroaryl is selected from the group consisting of 2-methyl-benzofuran-4-yl, 3-methyl-benzofuran-4-yl, 2,3-dimethyl-benzofuran-4-yl, 6-fluoro-2-methyl-benzofuran-4-yl, 1-methyl-1H-indazol-3-yl, benzoxazol-4-yl, 2-methyl-benzoxazol-7-yl, benzothiazol-7-yl, benzoisothiazol-3-yl, benzo[2,1,3]thiadiazol-4-yl, pyrrolo[2,1-b]thiazol-7-yl, imidazo[1,2-a]pyridin-3-yl, 4-chloro-pyridine-2-yl, 4-methoxy-pyridine-2-yl, 2-methoxy-pyridine-3-yl, 6-methyl-pyridine-2-yl, 6-methoxy-pyridine-2-yl, 6-trifluoromethyl-pyridine-2-yl, 2,5-dimethyl-oxazol-4-yl, 2,4-dimethyl-oxazol-5-yl, 2,4-dimethyl-thiazol-5-yl, 2-methyl-thiazol-4-yl, 4-methyl-thiazol-5-yl, 4-methyl-thiazol-2-yl, and 5-methyl-thiazol-2-yl. (In a sub-embodiment, in case R¹ represents heteroaryl, said heteroaryl is selected from the group consisting of 2-methyl-benzofuran-4-yl, 3-methyl-benzofuran-4-yl, 2,3-dimethyl-benzofuran-4-yl, 6-fluoro-2-methyl-benzofuran-4-yl, 1-methyl-1H-indazol-3-yl, benzoxazol-4-yl, 2-methyl-benzoxazol-7-yl, benzothiazol-7-yl, benzoisothiazol-3-yl, benzo[2,1,3]thiadiazol-4-yl, pyrrolo[2,1-b]thiazol-7-yl, and imidazo[1,2-a]pyridin-3-yl.)
19) A further embodiment of the invention relates to compounds of formula (I) according to any one of embodiments 1) to 13) or 15), wherein, in case R¹ represents heterocyclyl, said heterocyclyl is selected from the group consisting of 2,3-dihydro-benzofuran-4-yl and 2,3-dihydro-benzo[1,4]dioxin-5-yl.
20) A further embodiment of the invention relates to compounds of formula (I) according to any one of embodiments 1) to 12), wherein R¹ represents aryl, wherein the aryl is unsubstituted or mono-, di-, or tri-substituted, wherein the substituents are independently selected from the group consisting of (C₁₋₄)alkyl, (C₁₋₄)alkoxy, fluoroalkyl, fluoroalkoxy, and halogen
21) A further embodiment of the invention relates to compounds of formula (I) according to any one of embodiments 1) to 19), wherein, in case R¹ represents a bicyclic heteroaryl group or R¹ represents a heterocyclyl group, the bond with which said bicyclic heteroaryl or said heterocyclyl is attached to the rest of the molecule is positioned on an aromatic carbon atom of said group in alpha position to a bridgehead atom as further illustrated in the following examples:

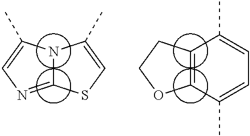

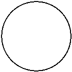

=bridgehead atom of the bicyclic ring system; [symbol]
=bond with which in these examples the heterocyclyl group may be attached to the rest of the molecule.
22) In another embodiment of the invention, examples of compounds of formula (I) according to embodiment 1) are selected from the group consisting of:
2-Methyl-benzofuran-4-carboxylic acid [3-(2-methyl-5-m-tolyl-thiazole-4-carbonyl)-thiazolidin-2-ylmethyl]-amide;
5-Chloro-2-methyl-benzofuran-4-carboxylic acid [3-(2-methyl-5-m-tolyl-thiazole-4-carbonyl)-thiazolidin-2-ylmethyl]-amide;
2-Methyl-benzooxazole-7-carboxylic acid [3-(2-methyl-5-m-tolyl-thiazole-4-carbonyl)-thiazolidin-2-ylmethyl]-amide;
Pyrrolo[2,1-b]thiazole-7-carboxylic acid [3-(2-methyl-5-m-tolyl-thiazole-4-carbonyl)-thiazolidin-2-ylmethyl]-amide;
Benzo[d]isoxazole-3-carboxylic acid [3-(2-methyl-5-m-tolyl-thiazole-4-carbonyl)-thiazolidin-2-ylmethyl]-amide;
7-Chloro-2-methyl-benzofuran-4-carboxylic acid [3-(2-methyl-5-m-tolyl-thiazole-4-carbonyl)-thiazolidin-2-ylmethyl]-amide;
3-Methyl-benzofuran-4-carboxylic acid [3-(2-methyl-5-m-tolyl-thiazole-4-carbonyl)-thiazolidin-2-ylmethyl]-amide;
2-Methyl-benzooxazole-4-carboxylic acid [3-(2-methyl-5-m-tolyl-thiazole-4-carbonyl)-thiazolidin-2-ylmethyl]-amide;
6-Methyl-pyrrolo[2,1-b]thiazole-7-carboxylic acid [3-(2-methyl-5-m-tolyl-thiazole-4-carbonyl)-thiazolidin-2-ylmethyl]-amide;
6-Methyl-imidazo[2,1-b]thiazole-5-carboxylic acid [3-(2-methyl-5-m-tolyl-thiazole-4-carbonyl)-thiazolidin-2-ylmethyl]-amide;
2-Methyl-7-trifluoromethyl-benzofuran-4-carboxylic acid [3-(2-methyl-5-m-tolyl-thiazole-4-carbonyl)-thiazolidin-2-ylmethyl]-amide;
2,3-Dimethyl-benzofuran-4-carboxylic acid [3-(2-methyl-5-m-tolyl-thiazole-4-carbonyl)-thiazolidin-2-ylmethyl]-amide;
Imidazo[1,2-a]pyridine-3-carboxylic acid [3-(2-methyl-5-m-tolyl-thiazole-4-carbonyl)-thiazolidin-2-ylmethyl]-amide;
Imidazo[2,1-b]thiazole-5-carboxylic acid [3-(2-methyl-5-m-tolyl-thiazole-4-carbonyl)-thiazolidin-2-ylmethyl]-amide;
7-Fluoro-2-methyl-benzofuran-4-carboxylic acid [3-(2-methyl-5-m-tolyl-thiazole-4-carbonyl)-thiazolidin-2-ylmethyl]-amide;
2,3-Dihydro-benzofuran-4-carboxylic acid [3-(2-methyl-5-m-tolyl-thiazole-4-carbonyl)-thiazolidin-2-ylmethyl]-amide;
Benzooxazole-4-carboxylic acid [3-(2-methyl-5-m-tolyl-thiazole-4-carbonyl)-thiazolidin-2-ylmethyl]-amide;
2-Methyl-imidazo[1,2-a]pyridine-3-carboxylic acid [3-(2-methyl-5-m-tolyl-thiazole-4-carbonyl)-thiazolidin-2-ylmethyl]-amide;
2-Methyl-6-trifluoromethyl-benzofuran-4-carboxylic acid [3-(2-methyl-5-m-tolyl-thiazole-4-carbonyl)-thiazolidin-2-ylmethyl]-amide;
6-Fluoro-2-methyl-benzofuran-4-carboxylic acid [3-(2-methyl-5-m-tolyl-thiazole-4-carbonyl)-thiazolidin-2-ylmethyl]-amide;
2,3-Dihydro-benzo[1,4]dioxine-5-carboxylic acid [3-(2-methyl-5-m-tolyl-thiazole-4-carbonyl)-thiazolidin-2-ylmethyl]-amide;
Benzo[1,2,5]oxadiazole-4-carboxylic acid [3-(2-methyl-5-m-tolyl-thiazole-4-carbonyl)-thiazolidin-2-ylmethyl]-amide;
1-Methyl-1H-indazole-3-carboxylic acid [3-(2-methyl-5-m-tolyl-thiazole-4-carbonyl)-thiazolidin-2-ylmethyl]-amide;
6-Chloro-2-methyl-benzofuran-4-carboxylic acid [3-(2-methyl-5-m-tolyl-thiazole-4-carbonyl)-thiazolidin-2-ylmethyl]-amide;
Benzooxazole-7-carboxylic acid [3-(2-methyl-5-m-tolyl-thiazole-4-carbonyl)-thiazolidin-2-ylmethyl]-amide;
Benzo[1,2,5]thiadiazole-4-carboxylic acid [3-(2-methyl-5-m-tolyl-thiazole-4-carbonyl)-thiazolidin-2-ylmethyl]-amide;
Benzo[d]isothiazole-3-carboxylic acid [3-(2-methyl-5-m-tolyl-thiazole-4-carbonyl)-thiazolidin-2-ylmethyl]-amide;
Benzothiazole-7-carboxylic acid [3-(2-methyl-5-m-tolyl-thiazole-4-carbonyl)-thiazolidin-2-ylmethyl]-amide;
2,3-Dihydro-benzofuran-4-carboxylic acid {3-[4-(3-chloro-phenyl)-2-methyl-thiazole-5-carbonyl]-thiazolidin-2-ylmethyl}-amide;
1-Methyl-1H-indazole-3-carboxylic acid {3-[4-(3-chloro-phenyl)-2-methyl-thiazole-5-carbonyl]-thiazolidin-2-ylmethyl}-amide;
Benzothiazole-7-carboxylic acid {3-[4-(3-methoxy-phenyl)-2-methyl-thiazole-5-carbonyl]-thiazolidin-2-ylmethyl}-amide;
2,3-Dihydro-benzofuran-4-carboxylic acid {3-[4-(3-methoxy-phenyl)-2-methyl-thiazole-5-carbonyl]-thiazolidin-2-ylmethyl}-amide;
1-Methyl-1H-indazole-3-carboxylic acid {3-[4-(3-methoxy-phenyl)-2-methyl-thiazole-5-carbonyl]-thiazolidin-2-ylmethyl}-amide;
Benzothiazole-7-carboxylic acid {3-[4-(3-chloro-phenyl)-2-methyl-thiazole-5-carbonyl]-thiazolidin-2-ylmethyl}-amide;
Imidazo[1,2-a]pyridine-3-carboxylic acid {3-[4-(3-chloro-phenyl)-2-methyl-thiazole-5-carbonyl]-thiazolidin-2-ylmethyl}-amide;
2,3-Dihydro-benzofuran-4-carboxylic acid {3-[4-(4-fluoro-phenyl)-2-methyl-thiazole-5-carbonyl]-thiazolidin-2-ylmethyl}-amide;
2,3-Dihydro-benzofuran-4-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid {3-[4-(3-chloro-phenyl)-2-methyl-thiazole-5-carbonyl]-thiazolidin-2-ylmethyl}-amide;
1-Methyl-1H-indazole-3-carboxylic acid {3-[4-(4-fluoro-phenyl)-2-methyl-thiazole-5-carbonyl]-thiazolidin-2-ylmethyl}-amide;
Benzothiazole-7-carboxylic acid {3-[4-(4-fluoro-phenyl)-2-methyl-thiazole-5-carbonyl]-thiazolidin-2-ylmethyl}-amide;
1-Methyl-1H-indazole-3-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid {3-[4-(3-methoxy-phenyl)-2-methyl-thiazole-5-carbonyl]-thiazolidin-2-ylmethyl}-amide;
2,3-Dihydro-benzo[1,4]dioxine-5-carboxylic acid {3-[4-(4-fluoro-phenyl)-2-methyl-thiazole-5-carbonyl]-thiazolidin-2-ylmethyl}-amide;
Imidazo[1,2-a]pyridine-3-carboxylic acid {3-[4-(3-methoxy-phenyl)-2-methyl-thiazole-5-carbonyl]-thiazolidin-2-ylmethyl}-amide;
Imidazo[1,2-a]pyridine-3-carboxylic acid {3-[4-(4-fluoro-phenyl)-2-methyl-thiazole-5-carbonyl]-thiazolidin-2-ylmethyl}-amide;
Imidazo[1,2-a]pyridine-3-carboxylic acid [3-(2-methyl-4-p-tolyl-thiazole-5-carbonyl)-thiazolidin-2-ylmethyl]-amide;
Benzothiazole-7-carboxylic acid {3-[2-methyl-4-(3-trifluoromethyl-phenyl)-thiazole-5-carbonyl]-thiazolidin-2-ylmethyl}-amide;
2,3-Dihydro-benzo[1,4]dioxine-5-carboxylic acid {3-[2-methyl-4-(3-trifluoromethyl-phenyl)-thiazole-5-carbonyl]-thiazolidin-2-ylmethyl}-amide;
1-Methyl-1H-indazole-3-carboxylic acid {3-[2-methyl-4-(3-trifluoromethyl-phenyl)-thiazole-5-carbonyl]-thiazolidin-2-ylmethyl}-amide;
Imidazo[1,2-a]pyridine-3-carboxylic acid {3-[2-methyl-4-(3-trifluoromethyl-phenyl)-thiazole-5-carbonyl]-thiazolidin-2-ylmethyl}-amide;
2,3-Dihydro-benzofuran-4-carboxylic acid [3-(2-methyl-5-phenyl-thiazole-4-carbonyl)-thiazolidin-2-ylmethyl]-amide;
2,3-Dihydro-benzo[1,4]dioxine-5-carboxylic acid {3-[5-(3-methoxy-phenyl)-2-methyl-thiazole-4-carbonyl]-thiazolidin-2-ylmethyl}-amide;
Benzothiazole-7-carboxylic acid {3-[5-(3-methoxy-phenyl)-2-methyl-thiazole-4-carbonyl]-thiazolidin-2-ylmethyl}-amide;
2,3-Dihydro-benzofuran-4-carboxylic acid {3-[5-(3-methoxy-phenyl)-2-methyl-thiazole-4-carbonyl]-thiazolidin-2-ylmethyl}-amide;
Imidazo[1,2-a]pyridine-3-carboxylic acid {3-[5-(3-methoxy-phenyl)-2-methyl-thiazole-4-carbonyl]-thiazolidin-2-ylmethyl}-amide;
2,3-Dihydro-benzo[1,4]dioxine-5-carboxylic acid [3-(2-methyl-5-phenyl-thiazole-4-carbonyl)-thiazolidin-2-ylmethyl]-amide;
2,3-Dihydro-benzofuran-4-carboxylic acid {3-[5-(3-chloro-phenyl)-2-methyl-thiazole-4-carbonyl]-thiazolidin-2-ylmethyl}-amide;
2,3-Dihydro-benzofuran-4-carboxylic acid {3-[2-methyl-5-(4-trifluoromethyl-phenyl)-thiazole-4-carbonyl]-thiazolidin-2-ylmethyl}-amide;
2,3-Dihydro-benzo[1,4]dioxine-5-carboxylic acid {3-[2-methyl-5-(4-trifluoromethyl-phenyl)-thiazole-4-carbonyl]-thiazolidin-2-ylmethyl}-amide;
1-Methyl-1H-indazole-3-carboxylic acid {3-[2-methyl-5-(4-trifluoromethyl-phenyl)-thiazole-4-carbonyl]-thiazolidin-2-ylmethyl}-amide;
Imidazo[1,2-a]pyridine-3-carboxylic acid {3-[2-methyl-5-(4-trifluoromethyl-phenyl)-thiazole-4-carbonyl]-thiazolidin-2-ylmethyl}-amide;
Chroman-8-carboxylic acid [3-(2-methyl-5-m-tolyl-thiazole-4-carbonyl)-thiazolidin-2-ylmethyl]-amide;
Chroman-5-carboxylic acid [3-(2-methyl-5-m-tolyl-thiazole-4-carbonyl)-thiazolidin-2-ylmethyl]-amide;
3,4-Dihydro-2H-benzo[1,4]oxazine-5-carboxylic acid [3-(2-methyl-5-m-tolyl-thiazole-4-carbonyl)-thiazolidin-2-ylmethyl]-amide;
3,4-Dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3,4-dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3,4-dihydro-2H-benzo[1,4]oxazine-5-carboxylic acid [3-(2-methyl-5-m-tolyl-thiazole-4-carbonyl)-thiazolidin-2-ylmethyl]-amide;
3-Oxo-3,4-dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3-oxo-3,4-dihydro-2H-benzo[1,4]oxazine-8-carboxylic acid [3-(2-methyl-5-m-tolyl-thiazole-4-carbonyl)-thiazolidin-2-ylmethyl]-amide;
2,3-Dihydro-benzofuran-4-carboxylic acid [3-(3′,4′-dimethyl-biphenyl-2-carbonyl)-thiazolidin-2-ylmethyl]-amide;
Benzothiazole-7-carboxylic acid [3-(3′,4′-dimethyl-biphenyl-2-carbonyl)-thiazolidin-2-ylmethyl]-amide;
2,3-Dihydro-benzo[1,4]dioxine-5-carboxylic acid [3-(3′,4′-dimethyl-biphenyl-2-carbonyl)-thiazolidin-2-ylmethyl]-amide;
2,3-Dihydro-benzofuran-4-carboxylic acid {3-[2-methyl-4-(3-trifluoromethyl-phenyl)-thiazole-5-carbonyl]-thiazolidin-2-ylmethyl}-amide;
2,3-Dihydro-benzo[1,4]dioxine-5-carboxylic acid {3-[5-(4-fluoro-phenyl)-2-methyl-thiazole-4-carbonyl]-thiazolidin-2-ylmethyl}-amide;
Imidazo[1,2-a]pyridine-3-carboxylic acid {3-[5-(4-fluoro-phenyl)-2-methyl-thiazole-4-carbonyl]-thiazolidin-2-ylmethyl}-amide;
2,3-Dihydro-benzofuran-4-carboxylic acid {3-[5-(4-fluoro-phenyl)-2-methyl-thiazole-4-carbonyl]-thiazolidin-2-ylmethyl}-amide;
Benzothiazole-7-carboxylic acid {3-[5-(4-fluoro-phenyl)-2-methyl-thiazole-4-carbonyl]-thiazolidin-2-ylmethyl}-amide;
1-Methyl-1H-indazole-3-carboxylic acid [3-(2-methyl-5-phenyl-thiazole-4-carbonyl)-thiazolidin-2-ylmethyl]-amide;
2,3-Dihydro-benzofuran-4-carboxylic acid [3-(2-methyl-5-m-tolyl-oxazole-4-carbonyl)-thiazolidin-2-ylmethyl]-amide;
2,3-Dihydro-benzo[1,4]dioxine-5-carboxylic acid [3-(2-methyl-5-m-tolyl-oxazole-4-carbonyl)-thiazolidin-2-ylmethyl]-amide;
1-Methyl-1H-indazole-3-carboxylic acid [3-(3′,4′-dimethyl-biphenyl-2-carbonyl)-thiazolidin-2-ylmethyl]-amide;
Benzothiazole-7-carboxylic acid [3-(2-methyl-5-m-tolyl-oxazole-4-carbonyl)-thiazolidin-2-ylmethyl]-amide;
Imidazo[1,2-a]pyridine-3-carboxylic acid [3-(2-methyl-5-m-tolyl-oxazole-4-carbonyl)-thiazolidin-2-ylmethyl]-amide;
2,3-Dihydro-benzofuran-4-carboxylic acid {3-[2-methyl-5-(3-trifluoromethoxy-phenyl)-oxazole-4-carbonyl]-thiazolidin-2-ylmethyl}-amide;
2,3-Dihydro-benzo[1,4]dioxine-5-carboxylic acid {3-[2-methyl-5-(3-trifluoromethoxy-phenyl)-oxazole-4-carbonyl]-thiazolidin-2-ylmethyl}-amide;
1-Methyl-1H-indazole-3-carboxylic acid {3-[2-methyl-5-(3-trifluoromethoxy-phenyl)-oxazole-4-carbonyl]-thiazolidin-2-ylmethyl}-amide;
Benzothiazole-7-carboxylic acid [3-(2-methyl-5-phenyl-oxazole-4-carbonyl)-thiazolidin-2-ylmethyl]-amide;
1-Methyl-1H-indazole-3-carboxylic acid [3-(2-methyl-5-phenyl-oxazole-4-carbonyl)-thiazolidin-2-ylmethyl]-amide;
2,3-Dihydro-benzo[1,4]dioxine-5-carboxylic acid [3-(2-methyl-5-phenyl-oxazole-4-carbonyl)-thiazolidin-2-ylmethyl]-amide;
2,3-Dihydro-benzofuran-4-carboxylic acid [3-(2-methyl-5-phenyl-oxazole-4-carbonyl)-thiazolidin-2-ylmethyl]-amide;
Imidazo[1,2-a]pyridine-3-carboxylic acid [3-(2-methyl-5-phenyl-oxazole-4-carbonyl)-thiazolidin-2-ylmethyl]-amide;
Benzothiazole-7-carboxylic acid {3-[5-(3-fluoro-phenyl)-2-methyl-oxazole-4-carbonyl]-thiazolidin-2-ylmethyl}-amide;
2,3-Dihydro-benzofuran-4-carboxylic acid {3-[5-(3-fluoro-phenyl)-2-methyl-oxazole-4-carbonyl]-thiazolidin-2-ylmethyl}-amide;
2,3-Dihydro-benzo[1,4]dioxine-5-carboxylic acid {3-[5-(3-fluoro-phenyl)-2-methyl-oxazole-4-carbonyl]-thiazolidin-2-ylmethyl}-amide;
1-Methyl-1H-indazole-3-carboxylic acid {3-[5-(3-fluoro-phenyl)-2-methyl-oxazole-4-carbonyl]-thiazolidin-2-ylmethyl}-amide;
Imidazo[1,2-a]pyridine-3-carboxylic acid {3-[5-(3-fluoro-phenyl)-2-methyl-oxazole-4-carbonyl]-thiazolidin-2-ylmethyl}-amide;
Benzothiazole-7-carboxylic acid {3-[5-(3-chloro-phenyl)-2-methyl-oxazole-4-carbonyl]-thiazolidin-2-ylmethyl}-amide;
1-Methyl-1H-indazole-3-carboxylic acid {3-[5-(3-chloro-phenyl)-2-methyl-oxazole-4-carbonyl]-thiazolidin-2-ylmethyl}-amide;
2,3-Dihydro-benzofuran-4-carboxylic acid {3-[5-(3-chloro-phenyl)-2-methyl-oxazole-4-carbonyl]-thiazolidin-2-ylmethyl}-amide;
2,3-Dihydro-benzo[1,4]dioxine-5-carboxylic acid {3-[5-(3-chloro-phenyl)-2-methyl-oxazole-4-carbonyl]-thiazolidin-2-ylmethyl}-amide;
Imidazo[1,2-a]pyridine-3-carboxylic acid {3-[5-(3-chloro-phenyl)-2-methyl-oxazole-4-carbonyl]-thiazolidin-2-ylmethyl}-amide;
2,3-Dihydro-benzofuran-4-carboxylic acid {3-[5-(3-methoxy-phenyl)-2-methyl-oxazole-4-carbonyl]-thiazolidin-2-ylmethyl}-amide;
Benzothiazole-7-carboxylic acid {3-[5-(3-methoxy-phenyl)-2-methyl-oxazole-4-carbonyl]-thiazolidin-2-ylmethyl}-amide;
2,3-Dihydro-benzo[1,4]dioxine-5-carboxylic acid {3-[5-(3-methoxy-phenyl)-2-methyl-oxazole-4-carbonyl]-thiazolidin-2-ylmethyl}-amide;
1-Methyl-1H-indazole-3-carboxylic acid {3-[5-(3-methoxy-phenyl)-2-methyl-oxazole-4-carbonyl]-thiazolidin-2-ylmethyl}-amide;
Imidazo[1,2-a]pyridine-3-carboxylic acid {3-[5-(3-methoxy-phenyl)-2-methyl-oxazole-4-carbonyl]-thiazolidin-2-ylmethyl}-amide;
2,3-Dihydro-benzofuran-4-carboxylic acid [3-(2-m-tolyl-thiophene-3-carbonyl)-thiazolidin-2-ylmethyl]-amide;
Benzothiazole-7-carboxylic acid [3-(2-m-tolyl-thiophene-3-carbonyl)-thiazolidin-2-ylmethyl]-amide;
2,3-Dihydro-benzo[1,4]dioxine-5-carboxylic acid [3-(2-m-tolyl-thiophene-3-carbonyl)-thiazolidin-2-ylmethyl]-amide;
Benzothiazole-7-carboxylic acid {3-[5-(3-fluoro-phenyl)-thiazole-4-carbonyl]-thiazolidin-2-ylmethyl}-amide;
1-Methyl-1H-indazole-3-carboxylic acid {3-[5-(3-fluoro-phenyl)-thiazole-4-carbonyl]-thiazolidin-2-ylmethyl}-amide;
2,3-Dihydro-benzofuran-4-carboxylic acid {3-[5-(3-fluoro-phenyl)-thiazole-4-carbonyl]-thiazolidin-2-ylmethyl}-amide;
2,3-Dihydro-benzo[1,4]dioxine-5-carboxylic acid {3-[5-(3-fluoro-phenyl)-thiazole-4-carbonyl]-thiazolidin-2-ylmethyl}-amide;
Imidazo[1,2-a]pyridine-3-carboxylic acid {3-[5-(3-fluoro-phenyl)-thiazole-4-carbonyl]-thiazolidin-2-ylmethyl}-amide;
2,3-Dihydro-benzofuran-4-carboxylic acid {3-[2-cyclopropyl-5-(3-fluoro-phenyl)-thiazole-4-carbonyl]-thiazolidin-2-ylmethyl}-amide;
2,3-Dihydro-benzo[1,4]dioxine-5-carboxylic acid {3-[2-cyclopropyl-5-(3-fluoro-phenyl)-thiazole-4-carbonyl]-thiazolidin-2-ylmethyl}-amide;
2,3-Dihydro-benzo[1,4]dioxine-5-carboxylic acid {3-[5-(3-fluoro-4-methyl-phenyl)-2-methyl-thiazole-4-carbonyl]-thiazolidin-2-ylmethyl}-amide;
2,3-Dihydro-benzofuran-4-carboxylic acid {3-[5-(3-fluoro-4-methyl-phenyl)-2-methyl-thiazole-4-carbonyl]-thiazolidin-2-ylmethyl}-amide;
Benzothiazole-7-carboxylic acid {3-[5-(3-fluoro-4-methyl-phenyl)-2-methyl-thiazole-4-carbonyl]-thiazolidin-2-ylmethyl}-amide;
2,3-Dihydro-benzofuran-4-carboxylic acid {3-[5-(3,4-difluoro-phenyl)-2-methyl-thiazole-4-carbonyl]-thiazolidin-2-ylmethyl}-amide;
2,3-Dihydro-benzo[1,4]dioxine-5-carboxylic acid {3-[5-(3,4-difluoro-phenyl)-2-methyl-thiazole-4-carbonyl]-thiazolidin-2-ylmethyl}-amide;
Benzothiazole-7-carboxylic acid {3-[5-(2,3-difluoro-4-methyl-phenyl)-2-methyl-thiazole-4-carbonyl]-thiazolidin-2-ylmethyl}-amide;
Imidazo[1,2-a]pyridine-3-carboxylic acid {3-[5-(2,3-difluoro-4-methyl-phenyl)-2-methyl-thiazole-4-carbonyl]-thiazolidin-2-ylmethyl}-amide;
2,3-Dihydro-benzofuran-4-carboxylic acid {3-[5-(2,3-difluoro-4-methyl-phenyl)-2-methyl-thiazole-4-carbonyl]-thiazolidin-2-ylmethyl}-amide;
1-Methyl-1H-indazole-3-carboxylic acid {3-[5-(2,3-difluoro-4-methyl-phenyl)-2-methyl-thiazole-4-carbonyl]-thiazolidin-2-ylmethyl}-amide;
2,3-Dihydro-benzo[1,4]dioxine-5-carboxylic acid {3-[5-(2,3-difluoro-4-methyl-phenyl)-2-methyl-thiazole-4-carbonyl]-thiazolidin-2-ylmethyl}-amide;
2,3-Dihydro-benzofuran-4-carboxylic acid [3-(2-dimethylamino-5-m-tolyl-thiazole-4-carbonyl)-thiazolidin-2-ylmethyl]-amide;
Imidazo[1,2-a]pyridine-3-carboxylic acid [3-(3′,4′-dimethyl-biphenyl-2-carbonyl)-thiazolidin-2-ylmethyl]-amide;
1H-Indazole-7-carboxylic acid [3-(2-methyl-5-m-tolyl-thiazole-4-carbonyl)-thiazolidin-2-ylmethyl]-amide;
6-Fluoro-4H-benzo[1,3]dioxine-8-carboxylic acid [3-(2-methyl-5-m-tolyl-thiazole-4-carbonyl)-thiazolidin-2-ylmethyl]-amide;
1-Methyl-1H-indole-4-carboxylic acid [3-(2-methyl-5-m-tolyl-thiazole-4-carbonyl)-thiazolidin-2-ylmethyl]-amide;
1-Methyl-1H-indole-7-carboxylic acid [3-(2-methyl-5-m-tolyl-thiazole-4-carbonyl)-thiazolidin-2-ylmethyl]-amide;
2,3-Dihydro-benzofuran-7-carboxylic acid [3-(2-methyl-5-m-tolyl-thiazole-4-carbonyl)-thiazolidin-2-ylmethyl]-amide;
1-Methyl-1H-indole-3-carboxylic acid [3-(2-methyl-5-m-tolyl-thiazole-4-carbonyl)-thiazolidin-2-ylmethyl]-amide;
5-Chloro-1,3-dimethyl-1H-pyrazole-4-carboxylic acid [3-(2-methyl-5-m-tolyl-thiazole-4-carbonyl)-thiazolidin-2-ylmethyl]-amide;
3-Ethyl-5-methyl-isoxazole-4-carboxylic acid [3-(2-methyl-5-m-tolyl-thiazole-4-carbonyl)-thiazolidin-2-ylmethyl]-amide;
1-Ethyl-3-methyl-1H-pyrazole-4-carboxylic acid [3-(2-methyl-5-m-tolyl-thiazole-4-carbonyl)-thiazolidin-2-ylmethyl]-amide;
1H-Benzoimidazole-4-carboxylic acid [3-(2-methyl-5-m-tolyl-thiazole-4-carbonyl)-thiazolidin-2-ylmethyl]-amide;
5-Ethyl-3-methyl-isoxazole-4-carboxylic acid [3-(2-methyl-5-m-tolyl-thiazole-4-carbonyl)-thiazolidin-2-ylmethyl]-amide;
3,5-Dimethyl-isoxazole-4-carboxylic acid [3-(2-methyl-5-m-tolyl-thiazole-4-carbonyl)-thiazolidin-2-ylmethyl]-amide;
1-Methyl-5-trifluoromethyl-1H-pyrazole-4-carboxylic acid [3-(2-methyl-5-m-tolyl-thiazole-4-carbonyl)-thiazolidin-2-ylmethyl]-amide;
1,3-Dimethyl-1H-pyrazole-4-carboxylic acid [3-(2-methyl-5-m-tolyl-thiazole-4-carbonyl)-thiazolidin-2-ylmethyl]-amide;
Benzo[1,2,3]thiadiazole-5-carboxylic acid [3-(2-methyl-5-m-tolyl-thiazole-4-carbonyl)-thiazolidin-2-ylmethyl]-amide;
Benzothiazole-6-carboxylic acid [3-(2-methyl-5-m-tolyl-thiazole-4-carbonyl)-thiazolidin-2-ylmethyl]-amide;
1,3,5-Trimethyl-1H-pyrazole-4-carboxylic acid [3-(2-methyl-5-m-tolyl-thiazole-4-carbonyl)-thiazolidin-2-ylmethyl]-amide;
2,2-Dimethyl-2,3-dihydro-benzofuran-7-carboxylic acid [3-(2-methyl-5-m-tolyl-thiazole-4-carbonyl)-thiazolidin-2-ylmethyl]-amide;
2,2-Difluoro-benzo[1,3]dioxole-4-carboxylic acid [3-(2-methyl-5-m-tolyl-thiazole-4-carbonyl)-thiazolidin-2-ylmethyl]-amide;
1-Methyl-1H-indole-5-carboxylic acid [3-(2-methyl-5-m-tolyl-thiazole-4-carbonyl)-thiazolidin-2-ylmethyl]-amide;
2,3-Dihydro-benzo[1,4]dioxine-6-carboxylic acid [3-(2-methyl-5-m-tolyl-thiazole-4-carbonyl)-thiazolidin-2-ylmethyl]-amide;
3H-Benzoimidazole-5-carboxylic acid [3-(2-methyl-5-m-tolyl-thiazole-4-carbonyl)-thiazolidin-2-ylmethyl]-amide;
1-Isopropyl-1H-pyrazole-4-carboxylic acid [3-(2-methyl-5-m-tolyl-thiazole-4-carbonyl)-thiazolidin-2-ylmethyl]-amide;
2-Methyl-benzothiazole-5-carboxylic acid [3-(2-methyl-5-m-tolyl-thiazole-4-carbonyl)-thiazolidin-2-ylmethyl]-amide;
1-Methyl-1H-benzotriazole-5-carboxylic acid [3-(2-methyl-5-m-tolyl-thiazole-4-carbonyl)-thiazolidin-2-ylmethyl]-amide; and
1-Methyl-1H-benzoimidazole-5-carboxylic acid [3-(2-methyl-5-m-tolyl-thiazole-4-carbonyl)-thiazolidin-2-ylmethyl]-amide;
   wherein it is well understood that the thiazolidin-2-ylmethyl moiety of the above listed compounds may be in absolute (R)- or (S)-configuration.
   23) In another embodiment of the invention, in addition to the compounds listed in embodiment 22), further examples of compounds of formula (I) according to embodiment 1) are selected from the group consisting of:
Quinoxaline-5-carboxylic acid [3-(2-methyl-5-m-tolyl-thiazole-4-carbonyl)-thiazolidin-2-ylmethyl]-amide;
1H-Indazole-4-carboxylic acid [3-(2-methyl-5-m-tolyl-thiazole-4-carbonyl)-thiazolidin-2-ylmethyl]-amide;
4-Chloro-pyridine-2-carboxylic acid [3-(2-methyl-5-m-tolyl-thiazole-4-carbonyl)-thiazolidin-2-ylmethyl]-amide;
4-Methoxy-pyridine-2-carboxylic acid [3-(2-methyl-5-m-tolyl-thiazole-4-carbonyl)-thiazolidin-2-ylmethyl]-amide;
2-Methoxy-N-[3-(2-methyl-5-m-tolyl-thiazole-4-carbonyl)-thiazolidin-2-ylmethyl]-nicotinamide;
6-Methyl-pyridine-2-carboxylic acid [3-(2-methyl-5-m-tolyl-thiazole-4-carbonyl)-thiazolidin-2-ylmethyl]-amide;
6-Trifluoromethyl-pyridine-2-carboxylic acid [3-(2-methyl-5-m-tolyl-thiazole-4-carbonyl)-thiazolidin-2-ylmethyl]-amide;
2,5-Dimethyl-oxazole-4-carboxylic acid [3-(2-methyl-5-m-tolyl-thiazole-4-carbonyl)-thiazolidin-2-ylmethyl]-amide;
6-Methoxy-pyridine-2-carboxylic acid [3-(2-methyl-5-m-tolyl-thiazole-4-carbonyl)-thiazolidin-2-ylmethyl]-amide;
2,4-Dimethyl-thiazole-5-carboxylic acid [3-(2-methyl-5-m-tolyl-thiazole-4-carbonyl)-thiazolidin-2-ylmethyl]-amide;
2-Methyl-thiazole-4-carboxylic acid [3-(2-methyl-5-m-tolyl-thiazole-4-carbonyl)-thiazolidin-2-ylmethyl]-amide;
4-Methyl-thiazole-5-carboxylic acid [3-(2-methyl-5-m-tolyl-thiazole-4-carbonyl)-thiazolidin-2-ylmethyl]-amide;
3-Methyl-quinoxaline-2-carboxylic acid [3-(2-methyl-5-m-tolyl-thiazole-4-carbonyl)-thiazolidin-2-ylmethyl]-amide;
5-Chloro-pyridine-2-carboxylic acid [3-(2-methyl-5-m-tolyl-thiazole-4-carbonyl)-thiazolidin-2-ylmethyl]-amide;
5-Methyl-thiazole-2-carboxylic acid [3-(2-methyl-5-m-tolyl-thiazole-4-carbonyl)-thiazolidin-2-ylmethyl]-amide;
2,6-Dimethoxy-N-[3-(2-methyl-5-m-tolyl-thiazole-4-carbonyl)-thiazolidin-2-ylmethyl]-nicotinamide;
Thiazole-4-carboxylic acid [3-(2-methyl-5-m-tolyl-thiazole-4-carbonyl)-thiazolidin-2-ylmethyl]-amide;
2,4-Dimethyl-oxazole-5-carboxylic acid [3-(2-methyl-5-m-tolyl-thiazole-4-carbonyl)-thiazolidin-2-ylmethyl]-amide;
4-Methyl-thiazole-2-carboxylic acid [3-(2-methyl-5-m-tolyl-thiazole-4-carbonyl)-thiazolidin-2-ylmethyl]-amide;
6-Methyl-N-[3-(2-methyl-5-m-tolyl-thiazole-4-carbonyl)-thiazolidin-2-ylmethyl]-nicotinamide;
5-Methyl-N-[3-(2-methyl-5-m-tolyl-thiazole-4-carbonyl)-thiazolidin-2-ylmethyl]-nicotinamide;
4-Methyl-oxazole-5-carboxylic acid [3-(2-methyl-5-m-tolyl-thiazole-4-carbonyl)-thiazolidin-2-ylmethyl]-amide;
[2,6]Naphthyridine-3-carboxylic acid [3-(2-methyl-5-m-tolyl-thiazole-4-carbonyl)-thiazolidin-2-ylmethyl]-amide;
[1,5]Naphthyridine-2-carboxylic acid [3-(2-methyl-5-m-tolyl-thiazole-4-carbonyl)-thiazolidin-2-ylmethyl]-amide;
Quinoxaline-2-carboxylic acid [3-(2-methyl-5-m-tolyl-thiazole-4-carbonyl)-thiazolidin-2-ylmethyl]-amide;
5-Methyl-isoxazole-3-carboxylic acid [3-(2-methyl-5-m-tolyl-thiazole-4-carbonyl)-thiazolidin-2-ylmethyl]-amide;
[1,8]Naphthyridine-2-carboxylic acid [3-(2-methyl-5-m-tolyl-thiazole-4-carbonyl)-thiazolidin-2-ylmethyl]-amide;
4-Methyl-N-[3-(2-methyl-5-m-tolyl-thiazole-4-carbonyl)-thiazolidin-2-ylmethyl]-nicotinamide;
N-[3-(2-Methyl-5-m-tolyl-thiazole-4-carbonyl)-thiazolidin-2-ylmethyl]-6-trifluoromethyl-nicotinamide;
1H-Pyrazolo[3,4-b]pyridine-5-carboxylic acid [3-(2-methyl-5-m-tolyl-thiazole-4-carbonyl)-thiazolidin-2-ylmethyl]-amide;
Oxazole-4-carboxylic acid [3-(2-methyl-5-m-tolyl-thiazole-4-carbonyl)-thiazolidin-2-ylmethyl]-amide;
N-[3-(2-Methyl-5-m-tolyl-thiazole-4-carbonyl)-thiazolidin-2-ylmethyl]-4-trifluoromethyl-nicotinamide;
1H-Pyrazolo[3,2-b]pyridine-6-carboxylic acid [3-(2-methyl-5-m-tolyl-thiazole-4-carbonyl)-thiazolidin-2-ylmethyl]-amide;
4-Chloro-N-[3-(2-methyl-5-m-tolyl-thiazole-4-carbonyl)-thiazolidin-2-ylmethyl]-nicotinamide;
Benzothiazole-7-carboxylic acid {3-[2-dimethylamino-5-(3-methoxy-phenyl)-thiazole-4-carbonyl]-thiazolidin-2-ylmethyl}-amide;
Benzothiazole-7-carboxylic acid {3-[2-dimethylamino-5-(4-fluoro-phenyl)-thiazole-4-carbonyl]-thiazolidin-2-ylmethyl}-amide;
Benzothiazole-7-carboxylic acid {3-[2-dimethylamino-5-(3-fluoro-phenyl)-thiazole-4-carbonyl]-thiazolidin-2-ylmethyl}-amide;
Benzothiazole-7-carboxylic acid {3-[2-dimethylamino-5-(3,4-dimethyl-phenyl)-thiazole-4-carbonyl]-thiazolidin-2-ylmethyl}-amide;
Benzothiazole-7-carboxylic acid [3-(3-m-tolyl-pyrazine-2-carbonyl)-thiazolidin-2-ylmethyl]-amide;
Benzothiazole-7-carboxylic acid {3-[3-(3,4-dimethyl-phenyl)-pyrazine-2-carbonyl]-thiazolidin-2-ylmethyl}-amide; and
Benzothiazole-7-carboxylic acid {3-[3-(3-methoxy-phenyl)-pyrazine-2-carbonyl]-thiazolidin-2-ylmethyl}-amide;
   wherein it is well understood that the thiazolidin-2-ylmethyl moiety of the above listed compounds may be in absolute (R)- or (S)-configuration.
   24) In another embodiment of the invention, in addition to the compounds listed in embodiments 22) and/or 23), further examples of compounds of formula (I) according to embodiment 1) are selected from the group consisting of:
2,3-Dihydro-benzofuran-4-carboxylic acid {3-[5-(3-chloro-phenyl)-thiazole-4-carbonyl]-thiazolidin-2-ylmethyl}-amide;
2,3-Dihydro-benzofuran-4-carboxylic acid {3-[5-(3,4-dimethyl-phenyl)-thiazole-4-carbonyl]-thiazolidin-2-ylmethyl}-amide;
2,3-Dihydro-benzofuran-4-carboxylic acid {3-[2-dimethylamino-5-(4-fluoro-phenyl)-thiazole-4-carbonyl]-thiazolidin-2-ylmethyl}-amide;
2,3-Dihydro-benzofuran-4-carboxylic acid {3-[2-dimethylamino-5-(2-fluoro-phenyl)-thiazole-4-carbonyl]-thiazolidin-2-ylmethyl}-amide;
2,3-Dihydro-benzofuran-4-carboxylic acid {3-[2-(ethyl-methyl-amino)-5-(3-methoxy-phenyl)-thiazole-4-carbonyl]-thiazolidin-2-ylmethyl}-amide;
2,3-Dihydro-benzofuran-4-carboxylic acid {3-[2-(ethyl-methyl-amino)-5-(4-fluoro-phenyl)-thiazole-4-carbonyl]-thiazolidin-2-ylmethyl}-amide;
2,3-Dihydro-benzofuran-4-carboxylic acid {3-[2-dimethylamino-5-(3-methoxy-phenyl)-thiazole-4-carbonyl]-thiazolidin-2-ylmethyl}-amide;
2,3-Dihydro-benzofuran-4-carboxylic acid {3-[5-(3-methoxy-phenyl)-thiazole-4-carbonyl]-thiazolidin-2-ylmethyl}-amide;
2,3-Dihydro-benzofuran-4-carboxylic acid [3-(5-m-tolyl-thiazole-4-carbonyl)-thiazolidin-2-ylmethyl]-amide;
2,3-Dihydro-benzofuran-4-carboxylic acid [3-(2-dimethylamino-5-p-tolyl-thiazole-4-carbonyl)-thiazolidin-2-ylmethyl]-amide;
2,3-Dihydro-benzofuran-4-carboxylic acid {3-[2-dimethylamino-5-(3-fluoro-4-methyl-phenyl)-thiazole-4-carbonyl]-thiazolidin-2-ylmethyl}-amide;
2,3-Dihydro-benzofuran-4-carboxylic acid {3-[5-(3-chloro-phenyl)-2-dimethylamino-thiazole-4-carbonyl]-thiazolidin-2-ylmethyl}-amide;
2,3-Dihydro-benzofuran-4-carboxylic acid {3-[3-(3,4-dimethyl-phenyl)-pyrazine-2-carbonyl]-thiazolidin-2-ylmethyl}-amide;
2,3-Dihydro-benzofuran-4-carboxylic acid {3-[3-(4-fluoro-3-methyl-phenyl)-pyrazine-2-carbonyl]-thiazolidin-2-ylmethyl}-amide;
Benzothiazole-7-carboxylic acid {3-[2-dimethylamino-5-(4-fluoro-phenyl)-thiazole-4-carbonyl]-thiazolidin-2-ylmethyl}-amide;
Benzothiazole-7-carboxylic acid {3-[2-dimethylamino-5-(3-methoxy-phenyl)-thiazole-4-carbonyl]-thiazolidin-2-ylmethyl}-amide;
Benzothiazole-7-carboxylic acid [3-(2-dimethylamino-5-p-tolyl-thiazole-4-carbonyl)-thiazolidin-2-ylmethyl]-amide;
Benzothiazole-7-carboxylic acid {3-[2-dimethylamino-5-(3-fluoro-4-methyl-phenyl)-thiazole-4-carbonyl]-thiazolidin-2-ylmethyl}-amide;
1-Methyl-1H-indole-3-carboxylic acid {3-[5-(4-fluoro-phenyl)-thiazole-4-carbonyl]-thiazolidin-2-ylmethyl}-amide;
1-Methyl-5-trifluoromethyl-1H-pyrazole-4-carboxylic acid {3-[5-(4-fluoro-phenyl)-thiazole-4-carbonyl]-thiazolidin-2-ylmethyl}-amide;
1-Methyl-1H-indole-3-carboxylic acid {3-[5-(3-fluoro-phenyl)-thiazole-4-carbonyl]-thiazolidin-2-ylmethyl}-amide;
1-Methyl-5-trifluoromethyl-1H-pyrazole-4-carboxylic acid {3-[5-(3-fluoro-phenyl)-thiazole-4-carbonyl]-thiazolidin-2-ylmethyl}-amide;
1-Ethyl-3-methyl-1H-pyrazole-4-carboxylic acid {3-[5-(3-fluoro-phenyl)-thiazole-4-carbonyl]-thiazolidin-2-ylmethyl}-amide;
1,3-Dimethyl-1H-pyrazole-4-carboxylic acid {3-[5-(3-fluoro-phenyl)-thiazole-4-carbonyl]-thiazolidin-2-ylmethyl}-amide; and
Quinoxaline-5-carboxylic acid {3-[5-(3-fluoro-phenyl)-thiazole-4-carbonyl]-thiazolidin-2-ylmethyl}-amide;
   wherein it is well understood that the thiazolidin-2-ylmethyl moiety of the above listed compounds may be in absolute (R)- or (S)-configuration.

The present invention also includes isotopically, especially ²H (deuterium) labelled compounds of formula (I) which compounds are identical to the compound of formula (I) wherein one or more atoms have been replaced by an atom having the same atomic number but an atomic mass different from the atomic mass usually found in nature. Isotopically labelled, especially ²H (deuterium) labelled compounds of formula (I) and salts thereof are within the scope of the present invention. Substitution of hydrogen with the heavier isotope ²H (deuterium) may lead to greater metabolic stability, resulting eg. in increased in-vivo half-life or reduced dosage requirements, or may lead to reduced inhibition of cytochrome P450 enzymes, resulting eg. in an improved safety profile. In one embodiment of the invention, the compounds of formula (I) are not isotopically labelled, or labelled with one or more deuterium atoms. In a sub-embodiment, the compounds of formula (I) are not isotopically labelled. Isotopically labelled compounds of formula (I) may be prepared in analogy to the methods described hereinafter, but using the appropriate isotopic variation of suitable reagents or starting materials.

The compounds of formula (I) may contain one or more stereogenic or asymmetric centers, such as one or more asymmetric carbon atoms. The compounds of formula (I) may thus be present as mixtures of stereoisomers or preferably as pure stereoisomers. Mixtures of stereoisomers may be separated in a manner known to a person skilled in the art.

Where the plural form is used for compounds, salts, pharmaceutical compositions, diseases and the like, this is intended to mean also a single compound, salt, or the like.

Any reference to a compound of formula (I) is to be understood as referring also to the salts (and especially the pharmaceutically acceptable salts) of such compounds, as appropriate and expedient.

The term “pharmaceutically acceptable salts” refers to non-toxic, inorganic or organic acid and/or base addition salts. Reference can be made to “Salt selection for basic drugs”, Int. J. Pharm. (1986), 33, 201-217.

The compounds of formula (I) and their pharmaceutically acceptable salts can be used as medicaments, e.g. in the form of pharmaceutical compositions for enteral or parenteral administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula (I) or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The present invention also relates to a method for the prevention or treatment of a disease or disorder mentioned herein comprising administering to a subject a pharmaceutically active amount of a compound of formula (I).

The compounds according to formula (I) are useful in the preparation of a medicament for the prevention or treatment of diseases selected from the group consisting of dysthymic disorders including major depression and cyclothymia, affective neurosis, all types of manic depressive disorders, delirium, psychotic disorders, schizophrenia, catatonic schizophrenia, delusional paranoia, adjustment disorders and all clusters of personality disorders; schizoaffective disorders; anxiety disorders including generalized anxiety, obsessive compulsive disorder, posttraumatic stress disorder, panic attacks, all types of phobic anxiety and avoidance; separation anxiety; all psychoactive substance use, abuse, seeking and reinstatement; all types of psychological or physical addictions, dissociative disorders including multiple personality syndromes and psychogenic amnesias; sexual and reproductive dysfunction; psychosexual dysfunction and addiction; tolerance to narcotics or withdrawal from narcotics; increased anaesthetic risk, anaesthetic responsiveness; hypothalamic-adrenal dysfunctions; disturbed biological and circadian rhythms; sleep disturbances associated with diseases such as neurological disorders including neuropathic pain and restless leg syndrome; sleep apnea; narcolepsy; chronic fatigue syndrome; insomnias related to psychiatric disorders; all types of idiopathic insomnias and parasomnias; sleep-wake schedule disorders including jet-lag; all dementias and cognitive dysfunctions in the healthy population and in psychiatric and neurological disorders; mental dysfunctions of aging; all types of amnesia; severe mental retardation; dyskinesias and muscular diseases; muscle spasticity, tremors, movement disorders; spontaneous and medication-induced dyskinesias; neurodegenerative disorders including Huntington's, Creutzfeld-Jacob's, Alzheimer's diseases and Tourette syndrome; Amyotrophic lateral sclerosis; Parkinson's disease; Cushing's syndrome; traumatic lesions; spinal cord trauma; head trauma; perinatal hypoxia; hearing loss; tinnitus; demyelinating diseases; spinal and cranial nerve diseases; ocular damage; retinopathy; epilepsy; seizure disorders; absence seizures, complex partial and generalized seizures; Lennox-Gastaut syndrome; migraine and headache; pain disorders; anaesthesia and analgesia; enhanced or exaggerated sensitivity to pain such as hyperalgesia, causalgia, and allodynia; acute pain; burn pain; atypical facial pain; neuropathic pain; back pain; complex regional pain syndrome I and II; arthritic pain; sports injury pain; dental pain; pain related to infection e.g. by HIV; post-chemotherapy pain; post-stroke pain; post-operative pain; neuralgia; osteoarthritis; conditions associated with visceral pain such as irritable bowel syndrome; eating disorders; diabetes; toxic and dysmetabolic disorders including cerebral anoxia, diabetic neuropathies and alcoholism; appetite, taste, eating, or drinking disorders; somatoform disorders including hypochondriasis; vomiting/nausea; emesis; gastric dyskinesia; gastric ulcers; Kallman's syndrome (anosmia); impaired glucose tolerance; intestinal motility dyskinesias; hypothalamic diseases; hypophysis diseases; hyperthermia syndromes, pyrexia, febrile seizures, idiopathic growth deficiency; dwarfism; gigantism; acromegaly; basophil adenoma; prolactinoma; hyperprolactinemia; brain tumors, adenomas; benign prostatic hypertrophy, prostate cancer; endometrial, breast, colon cancer; all types of testicular dysfunctions, fertility control; reproductive hormone abnormalities; hot flashes; hypothalamic hypogonadism, functional or psychogenic amenorrhea; urinary bladder incontinence; asthma; allergies; all types of dermatitis, acne and cysts, sebaceous gland dysfunctions; cardiovascular disorders; heart and lung diseases, acute and congestive heart failure; hypotension; hypertension; dyslipidemias, hyperlipidemias, insulin resistance; urinary retention; osteoporosis; angina pectoris; myocardial infarction; arrhythmias, coronary diseases, left ventricular hypertrophy; ischemic or haemorrhagic stroke; all types of cerebrovascular disorders including subarachnoid haemorrhage, ischemic and hemorrhagic stroke and vascular dementia; chronic renal failure and other renal diseases; gout; kidney cancer; urinary incontinence; and other diseases related to general orexin system dysfunctions.

Compounds of formula (I) are particularly suitable for use in the treatment of diseases or disorders selected from the group consisting of all types of sleep disorders, of stress-related syndromes, of psychoactive substance use, abuse, seeking and reinstatement, of cognitive dysfunctions in the healthy population and in psychiatric and neurologic disorders, of eating or drinking disorders. Eating disorders may be defined as comprising metabolic dysfunction; dysregulated appetite control; compulsive obesities; emeto-bulimia or anorexia nervosa. Pathologically modified food intake may result from disturbed appetite (attraction or aversion for food); altered energy balance (intake vs. expenditure); disturbed perception of food quality (high fat or carbohydrates, high palatability); disturbed food availability (unrestricted diet or deprivation) or disrupted water balance. Drinking disorders include polydipsias in psychiatric disorders and all other types of excessive fluid intake. Sleep disorders include all types of parasomnias, insomnias, narcolepsy and other disorders of excessive sleepiness, sleep-related dystonias; restless leg syndrome; sleep apneas; jet-lag syndrome; shift-work syndrome, delayed or advanced sleep phase syndrome or insomnias related to psychiatric disorders. Insomnias are defined as comprising sleep disorders associated with aging; intermittent treatment of chronic insomnia; situational transient insomnia (new environment, noise) or short-term insomnia due to stress; grief; pain or illness. Insomnia also include stress-related syndromes including post-traumatic stress disorders as well as other types and subtypes of anxiety disorders such as generalized anxiety, obsessive compulsive disorder, panic attacks and all types of phobic anxiety and avoidance. Psychoactive substance use, abuse, seeking and reinstatement are defined as all types of psychological or physical addictions and their related tolerance and dependence components. Cognitive dysfunctions include deficits in all types of attention, learning and memory functions occurring transiently or chronically in the normal, healthy, young, adult or aging population, and also occurring transiently or chronically in psychiatric, neurologic, cardiovascular and immune disorders.

In a further preferred embodiment of the invention compounds of formula (I) are particularly suitable for use in the treatment of diseases or disorders selected from the group consisting of sleep disorders that comprises all types of insomnias, narcolepsy and other disorders of excessive sleepiness, sleep-related dystonias, restless leg syndrome, sleep apneas, jet-lag syndrome, shift-work syndrome, delayed or advanced sleep phase syndrome or insomnias related to psychiatric disorders.

In another preferred embodiment of the invention compounds of formula (I) are particularly suitable for use in the treatment of diseases or disorders selected from the group consisting of cognitive dysfunctions that comprise deficits in all types of attention, learning and memory functions occurring transiently or chronically in the normal, healthy, young, adult or aging population, and also occurring transiently or chronically in psychiatric, neurologic, cardiovascular and immune disorders.

In another preferred embodiment of the invention compounds of formula (I) are particularly suitable for use in the treatment of diseases or disorders selected from the group consisting of eating disorders that comprise metabolic dysfunction; dysregulated appetite control; compulsive obesities; emeto-bulimia or anorexia nervosa.

In another preferred embodiment of the invention compounds of formula (I) are particularly suitable for use in the treatment of diseases or disorders selected from the group consisting of psychoactive substance use, abuse, seeking and reinstatement that comprise all types of psychological or physical addictions and their related tolerance and dependence components.

### Preparation of Compounds of Formula (I):

A further aspect of the invention is a process for the preparation of compounds of formula (I). Compounds according to formula (I) of the present invention can be prepared according to the general sequence of reactions outlined in the schemes below wherein A, B and R¹ are as defined for formula (I). The compounds obtained may also be converted into salts thereof in a manner known per se.

In general, all chemical transformations can be performed according to well-known standard methodologies as described in the literature or as described in the procedures or in the experimental part below.

Thiazolidine derivatives of formula (I) may be prepared according to scheme 1.

Deprotection of phthalimidoacetaldehyde diethylacetal (1) by reaction with 6N HCl in an aprotic solvent such as THF at rt affords the desired aldehyde (2). Cyclisation by reaction with 2-aminoethanthiol hydrochloride (3) in the presence of potassium acetate in a mixture water/EtOH gives the thiazolidine derivative (4). Acylation with an acid of formula B-A-COOH (e.g. TBTU, DIPEA, DMF, rt) furnishes the intermediate (5). Cleavage of the phthalimide protecting group e.g. by reaction with hydrazine monohydrate in refluxing EtOH, followed by acylation with an acid of the formula R¹—COOH (e.g. TBTU, DIPEA, DMF, rt) furnishes the thiazolidine derivatives of formula (I). Acids of formula B-A-COOH and R¹—COOH are commercially available, synthesized according to methods described below or by the methods given in the experimental part or analogous methods.

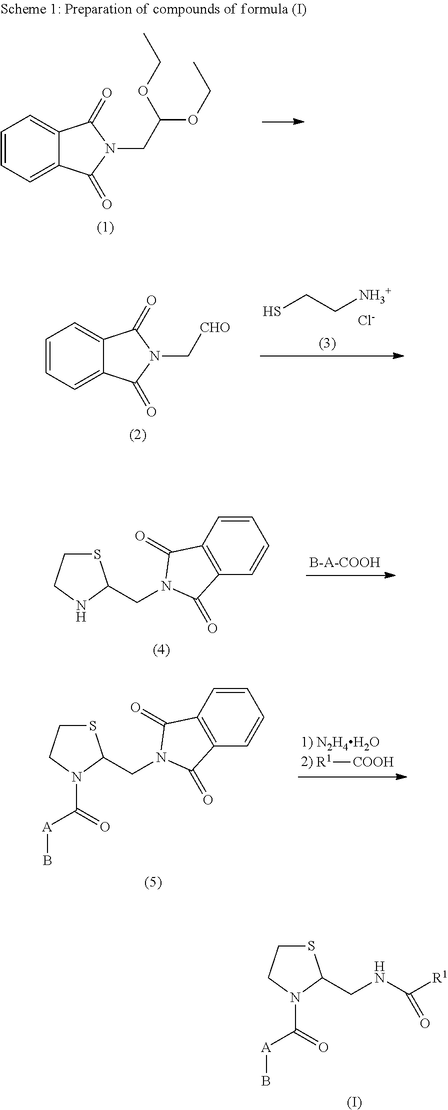

### Preparation of Carboxylic Acids B-A-COOH

Carboxylic acid derivatives B-A-COOH wherein B-A represents a thiazole-4-yl derivative are commercially available or can be synthesised according to scheme 2.

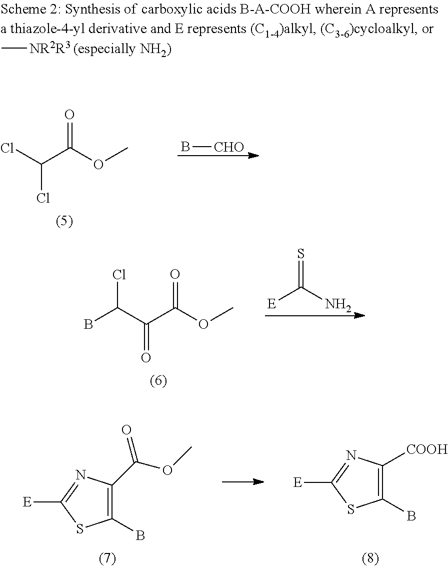

By reaction of methyl dichloroacetate (5) with an aldehyde of the formula B—CHO in the presence of a base such as KOtBu in an aprotic polar solvent such as THF at rt 3-chloro-2-oxo-propionic acid ester derivatives (6) are obtained. Compounds of structure (6) can be transformed by reaction with commercially available thioamide or thiourea derivatives E-C(S)—NH₂ at rt in solvents such as MeCN to provide thiazol-4-carboxylic acid ester derivatives (7). 2-Bromo-thiazole derivatives may be obtained by reaction of the respective 2-amino-thiazole derivative with isoamylnitrite in the presence of copper(II)bromide. At this stage, the bromo substituent may be removed via hydrogenation, or replaced with amines HNR²R³, sodium alkoxides or a CF₃ group (e.g. TMS-CF₃, CuI, KF, DMF, NMP; see T. Mano, Bioorg. Med. Chem. 2003, 11, 3879-3887). Saponification of the ester function of (7) using methods known in the art (e.g. KOH, EtOH; NaOH, EtOH; or NaOH, EtOH/water) provides the corresponding thiazol-4-carboxylic acid derivatives (8). Aldehydes of formula B—CHO are commercially available or well known in the art. (C₃₋₆)Cycloalkyl-thioamides may be synthesized by treatment of (C₃₋₆)cycloalkyl-carboxamides with Lawesson's reagent.

Carboxylic acid derivatives B-A-COOH wherein B-A represents a thiazole-5-yl derivative are commercially available or synthesised according to scheme 3.

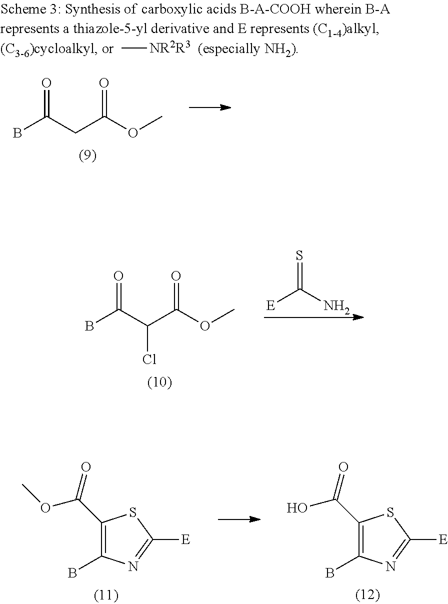

By refluxing a commercially available 3-oxo-propionic acid ester derivative (9) with SO₂Cl₂ in a solvent such as CHCl₃ the corresponding 2-chloro-3-oxo-propionic acid ester derivatives (10) can be obtained. Compounds of structure (10) can be transformed by reaction with commercially available thioamides E-C(S)—NH₂ at reflux temperature in solvents such as THF in presence of a base such as NaHCO₃ to the corresponding thiazol-5-carboxylic acid ester derivatives (11). Saponification of the ester function using methods known in the art (e.g. KOH, EtOH) provides the corresponding thiazol-5-carboxylic acid derivatives (12).

Carboxylic acid derivatives B-A-COOH wherein B-A represents an oxazole-4-yl derivative are commercially available or synthesised according to scheme 4.

By reaction of a commercially available 3-oxo-propionic acid ester derivative (13) with an aq. solution sodium nitrite in presence of an acid such as glacial acetic acid the corresponding oxime derivative (14) can be obtained. The 2-acetamido-3-oxo-propionic acid ester derivative (15) can be synthesized from compounds of structure (14) using acetic anhydride in presence of an acid such as glacial acetic acid and catalytic amounts of metal chlorides such as mercury chloride and zinc powder. Cyclization to the corresponding oxazole-4-carboxylic acid ester derivative (16) can be achieved under dehydrating conditions such as SOCl₂ in CHCl₃. Saponification of the ester function using methods known in the art (e.g. NaOH, EtOH/water) provides the corresponding oxazole-4 carboxylic acid derivative (17).

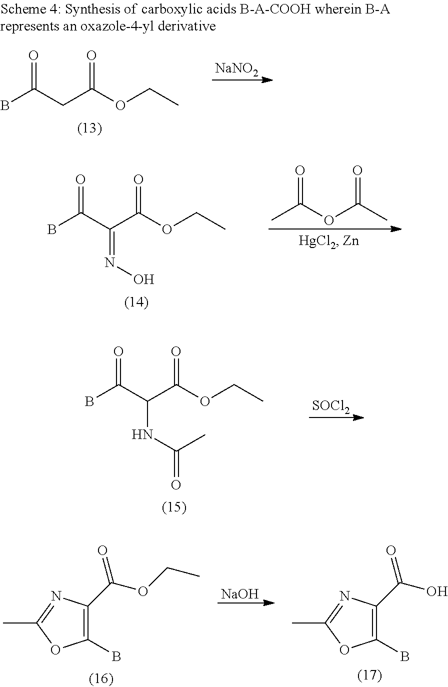

Carboxylic acid derivatives B-A-COOH wherein B-A represents a phenyl-2-yl derivative are commercially available or can be synthesised according to scheme 5.

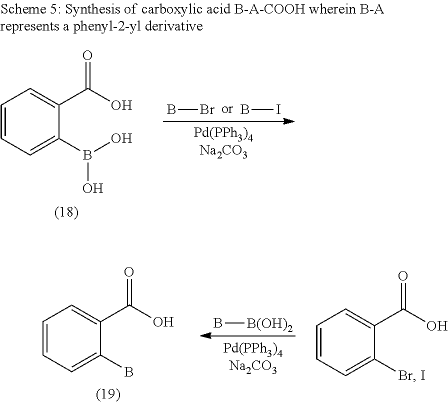

Reaction of commercially available (2-carboxyphenyl)-boronic acid derivatives (18) or esters thereof with commercially available aryl-bromides or aryl-iodides of formula B—Br or B—I in presence of a catalyst such as Pd(PPh₃)₄ and a base such as Na₂CO₃ under heating in a solvent such as toluene, dioxane, THF provides, after saponification, if needed, of the ester using well known methods, the corresponding phenyl-2-carboxylic acid derivatives (19). Alternatively, reaction of commercially available 2-bromo-, or 2-iodo-benzoic acid, or esters thereof, with commercially available boronic acid derivatives of formula B—B(OH)₂ using the conditions described before provides the corresponding phenyl-2-carboxylic acid derivatives (19).

Carboxylic acid derivatives B-A-COOH wherein B-A represents a pyrazine-2-yl derivative are commercially available or can be synthesised according to scheme 6.

Reaction of commercially available 3-chloro-pyrazine-2-carbonitrile (20) with commercially available boronic acids of formula B—B(OH)₂ in presence of a catalyst such as Pd(OAc)₂ and a base such as K₂CO₃ under heating in a solvent such as DME provides 3-(hetero)aryl-pyrazine-2-carbonitrile derivatives (21). Hydrolysis of (21) in the presence of a base such as NaOH in a alcoholic solvent such as MeOH furnishes the desired 3-(hetero)aryl-pyrazine-2-carboxylic acid derivatives (22).

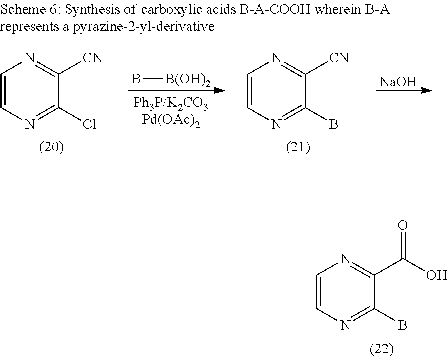

### Synthesis of Carboxylic Acids R¹—COOH

Carboxylic acids of formula R¹—COOH are commercially available or well known in the art (Lit. e.g. WO2001/96302; T. Eicher, S. Hauptmann “The chemistry of Heterocycles: Structure, Reactions, Syntheses, and Applications”, 2nd Edition 2003, Wiley, ISBN 978-3-527-30720-3; A. R. Katrizky, C. W. Rees, E. F. V. Scriven (Eds.) “Comprehensive Heterocyclic Chemistry II” 1996, Elsevier, ISBN 0-08-042072-9).

Carboxylic acid derivatives R¹—COOH which represent an imidazo[2,1-b]thiazole-2-carboxylic acid derivative are commercially available or can be synthesised according to scheme 7.

Pathway A: By reaction of 2-chloro-3-oxo-butyric acid methyl ester (23) with thiourea the amino-thiazole (24) can be obtained. Transformation to ester (25) can be accomplished with bromoacetaldehyde which can be generated in-situ from bromoacetaldehyde diethylacetal under acidic conditions. After saponification with bases such as NaOH the desired acid (26) can be obtained.

Pathway B: By heating a compound of structure (27) with N,N-dimethylformamide dimethylacetal in a solvent such as toluene formamidine derivatives (28) can be obtained. They can be alkylated with ethyl bromoacetate yielding the respective thiazolium bromide (29) which can be cyclised with strong bases such as DBU to the ester (30). Saponification of the ester function (e.g. NaOH, EtOH/water) provides the corresponding imidazo[2,1-b]thiazole-2-carboxylic acid derivatives (31).

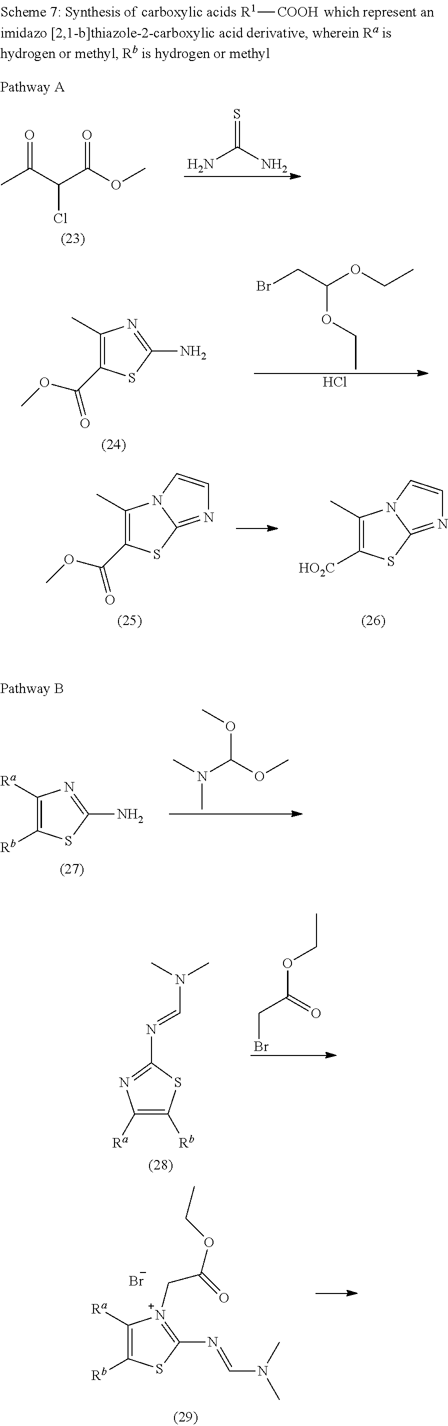

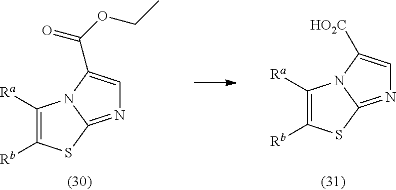

Carboxylic acid derivatives R¹—COOH which represent a pyrrolo[2,1-b]thiazole-7-carboxylic acid derivative can be synthesised according to scheme 8.

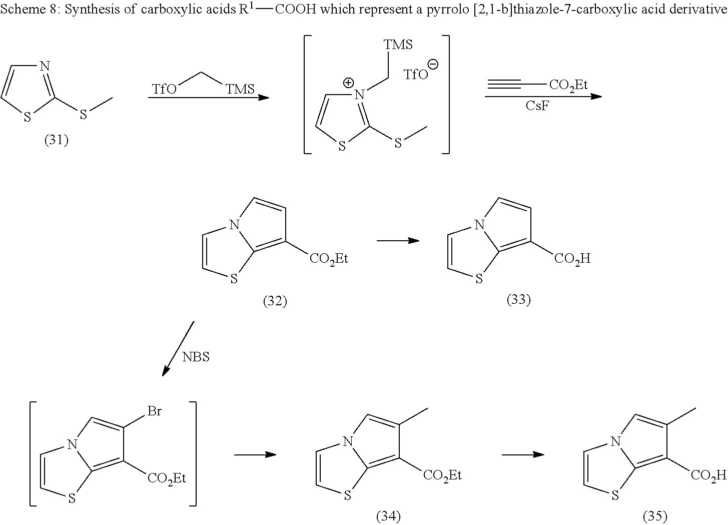

By reaction of 2-methylsulfanylthiazole (31) with trimethylsilylmethyl trifluoromethanesulfonate followed by cyclisation of the resulting thiazolinium salt by reaction with ethyl propiolate in the presence of caesium fluoride, the pyrrolo[2,1-b]thiazole (32) can be obtained. Saponification of the ester function (e.g. KOH, EtOH or NaOH, EtOH/water) provides the corresponding pyrrolo[2,1-b]thiazole-7-carboxylic acid derivative (33) (Berry C. R. et al., Organic Letters, 2007, 9, 21, 4099-4102).

Bromination of (32) by reaction with NBS followed by methylation of the resulting crude ethyl 6-bromo-pyrrolo[2,1-b]thiazole-7-carboxylate by reaction with dimethylzinc in the presence of a palladium catalyst such as Pd(dppf)Cl₂ gives the ester (34). Saponification of the ester function (e.g. NaOH, EtOH/water) provides the corresponding 6-methyl-pyrrolo[2,1-b]thiazole-7-carboxylic acid derivative (35).

Carboxylic acid derivatives R¹—COOH which represent a 3,4-dihydro-2H-benzo[1,4]oxazinyl- or 3-oxo-3,4-dihydro-2H-benzo[1,4]oxazinyl-carboxylic acid derivative can be synthesised according to the literature according to schemes 9 and 10.

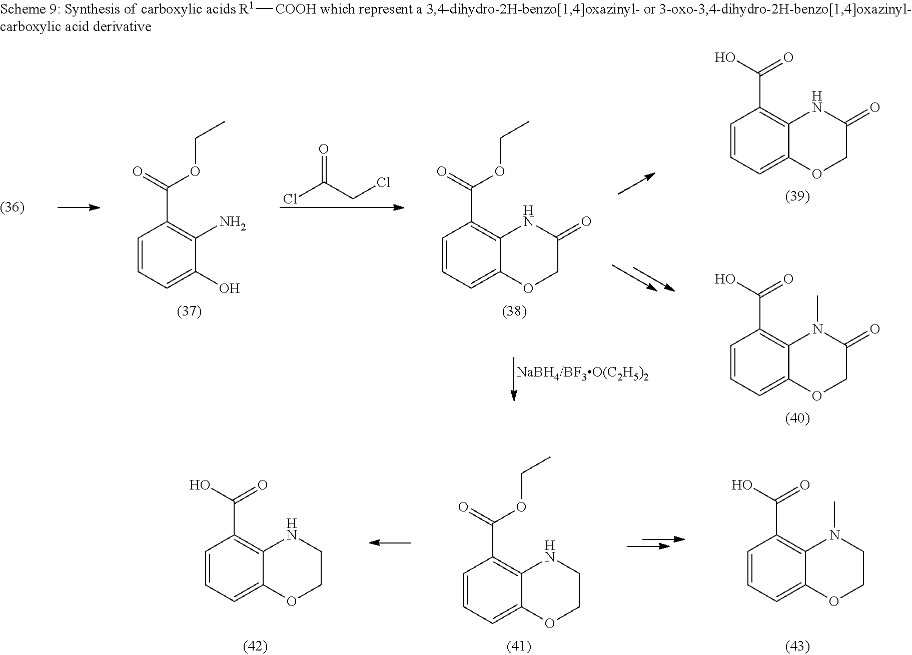

Esterification of 3-hydroxy-anthranilic acid (36) with conc. H₂SO₄ in EtOH provides the corresponding ethyl ester (37). Cyclisation with acetyl chloride in presence of a base such as K₂CO₃ in a solvent such as DMF provides 3-oxo-3,4-dihydro-2H-benzo[1,4]oxazine derivatives (38). Compounds of structure (36) can optionally be alkylated with alkylating reagents such as methyl iodide in presence of a base such as K₂CO₃. Saponification (e.g. NaOH, EtOH/water) leads to the corresponding acids (39) or (40). Reduction of compounds of structure (38) with NaBH₄ in the presence of BF₃-diethyl etherate leads to the corresponding 3,4-dihydro-2H-benzo[1,4]oxazine derivative (41) which can optionally be alkylated and/or saponified as described before to provide the corresponding acids (42) or (43) (Kuroita T. et al, Chemical Pharmaceutical Bulletin 1996, 44, 4, 756-764).

Hydrogenation of methyl 3-nitrosalicylate (44) in presence of a palladium catalyst provides the aniline derivative (45) which can be cyclized with chloroacetyl chloride as described before to the ester (46). Reduction of compounds of structure (46) with NaBH₄ in the presence of BF₃-diethyl etherate leads to the corresponding 3,4-dihydro-2H-benzo[1,4]oxazine derivative which can optionally be alkylated and/or saponified as described before to provide the corresponding acids (47) or (48) (Kuroita T. et al, Chemical Pharmaceutical Bulletin 1996, 44, 4, 756-764).

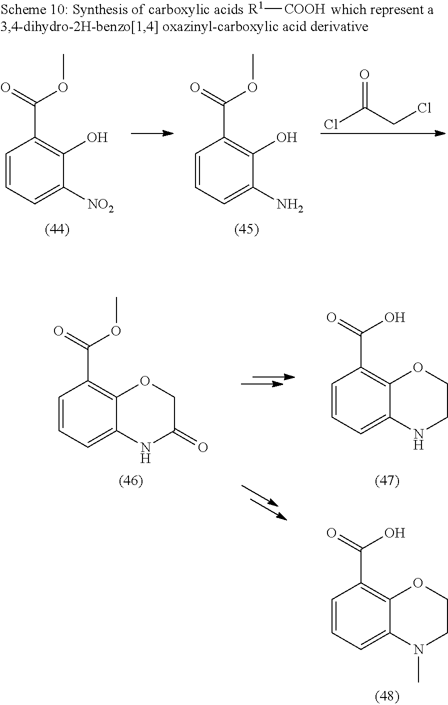

Carboxylic acid derivatives R¹—COOH which represent a benzooxazole-4-carboxylic acid derivative can be synthesised according to the literature according to schemes 11 and 12.

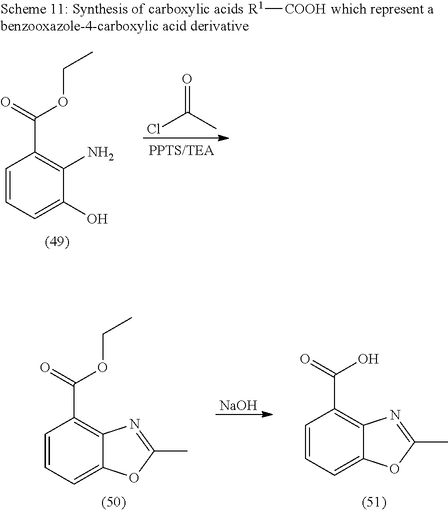

By cyclisation of ethyl 2-amino-3-hydroxybenzoate (49) with acetyl chloride in the presence of PPTS and TEA, the ester (50) can be obtained (Goldstein S. W. et al, Journal of Heterocyclic Chemistry, 1990, 27, 335-336). Saponification of the ester function (e.g. NaOH, EtOH/water) provides the corresponding 2-methyl-benzooxazole-4-carboxylic acid derivative (51).

By cyclisation of 3-aminosalicylic acid (52) with triethyl orthoformate in the presence of PTSA, the benzooxazole-7-carboxylic acid (53) can be obtained (WO2006/069155)

By cyclisation of 3-aminosalicylic acid (52) with triethyl orthoacetate in the presence of PTSA, the 2-methyl-benzooxazole-7-carboxylic acid (54) can be obtained (WO2006/069155)

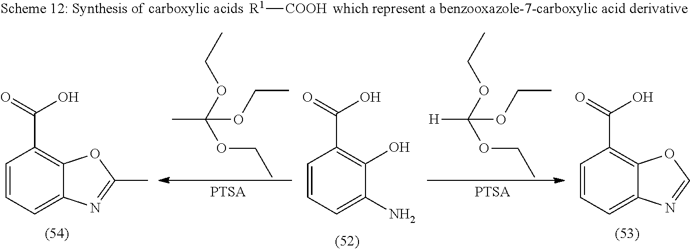

Carboxylic acid derivatives R¹—COOH which represent a benzothiazole-7-carboxylic acid derivative can be synthesised according to the literature according to scheme 13.

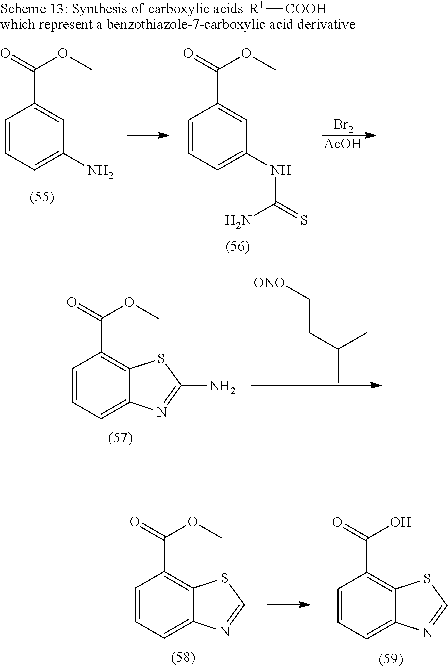

By reaction of methyl 3-aminobenzoate (55) with potassium thiocyanate in the presence of sulfuric acid and crown-ether 18-C-6, the thiourea (56) can be obtained. Cyclisation by reaction with bromine in acetic acid provides the 2-aminobenzothiazole derivative (57). Cleavage of the amino group by reaction with isoamyl nitrite furnishes the ester (58) (WO2005/092890). Saponification of the ester function (e.g. NaOH, MeOH/water) provides the corresponding benzothiazole-7-carboxylic acid derivative (59).

Carboxylic acid derivatives R¹—COOH which represent a benzofuran-4-carboxylic acid derivative can be synthesised according to the literature according to schemes 14 and 15.

By reaction of methyl 3-hydroxybenzoate (60) with 3-chloro-2-butanone, the ester (61) can be obtained. Cyclisation with sulfuric acid provides the 2,3-dimethylbenzofuran derivative (62) (Kawase Y. et al, Bulletin of the Chemical Society of Japan, 1967, 40, 5, 1224-1231. Saponification of the ester function using methods known in the art such as treatment with a base such as NaOH in a solvent such as MeOH/water provides the corresponding 2,3-dimethylbenzofuran-4-carboxylic acid derivative (63). On the other hand, reaction of methyl 3-hydroxybenzoate (60) with crotyl bromide furnishes the ester (64) which after reaction in N,N-dimethylaniline provides the ester (65). Ozonolysis followed by reaction with PTSA gives the 3-methylbenzofuran derivative (66) (Mohamadi F. et al, Journal of Medicinal Chemistry, 1994, 37, 232-239 and EP58906). Saponification of the ester function (e.g. NaOH, MeOH/water) provides the corresponding 3-methylbenzofuran-4-carboxylic acid derivative (67).

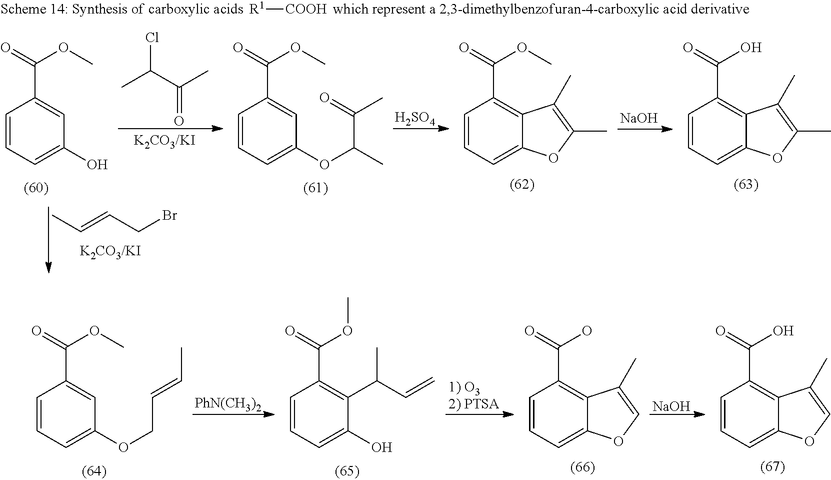

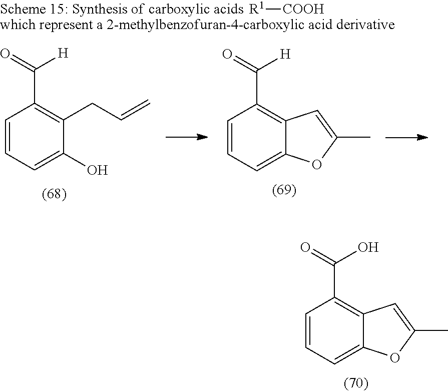

By cyclisation of 2-allyl-3-hydroxybenzaldehyde (68) with a palladium catalyst such as bis(acetonitrile)dichloropalladium in the presence of 1,4-benzoquinone and lithium chloride, the 2-methylbenzofuran carbaldehyde (69) can be obtained (Danheiser R. L. et al, Organic Letters, 2005, 7, 18, 3905-3908). Oxidation of the aldehyde function with sodium chlorite in the presence of a scavenger such as 2-methyl-2-butene furnishes the corresponding 2-methylbenzofuran-4-carboxylic acid (70).

Carboxylic acid derivatives R¹—COOH which represent a benzofuran-4-carboxylic acid derivative, wherein R represents one or two substituents selected from Cl, F and CF₃, can be synthesised according to the literature or according to scheme 16.

By esterification of phenol derivative (71) with EtOH in the presence of an acid such as sulfuric acid followed by allylation by reaction with allyl bromide in the presence of a K₂CO₃ and KI, the alkyl-ether derivative (72) can be obtained. Claisen rearrangement by reaction with N,N-dimethylaniline furnishes the phenol derivative (73). Ozonolysis followed by reaction with PTSA provides the benzofuran derivative (74). Saponification of the ester function of (74) using methods known in the art such as treatment with a base such as NaOH in a solvent such as EtOH/water provide the corresponding benzofuran-4-carboxylic acid derivatives (75). Furthermore, cyclisation of (73) with a palladium catalyst such as bis(acetonitrile)dichloropalladium in the presence of 1,4-benzoquinone and LiCl, the 2-methylbenzofuran derivative (76) can be obtained (Danheiser R. L. et al, Organic Letters, 2005, 7, 18, 3905-3908). Saponification of the ester function of (76) (e.g. NaOH, EtOH/water) provides the corresponding 2-methyl-benzofuran-4-carboxylic acid derivatives (77).

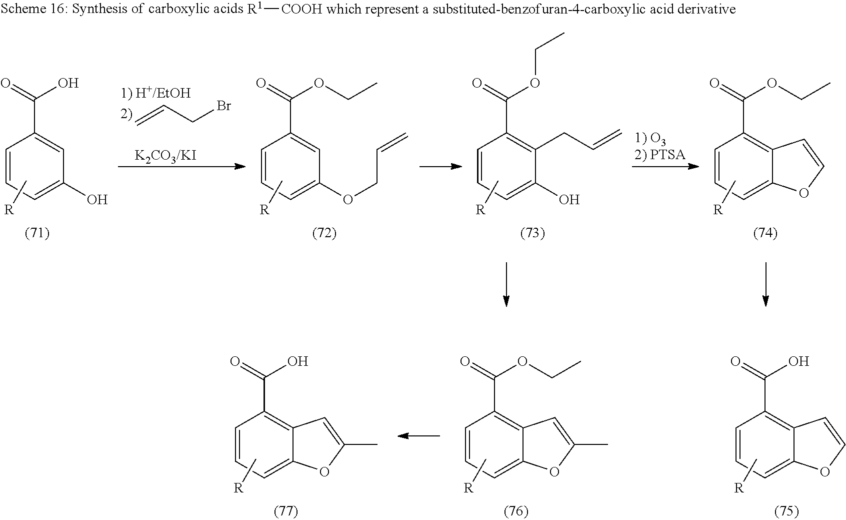

Derivatives of formula R¹—COOH wherein R¹ is chroman may be for instance synthesised according to scheme 17.

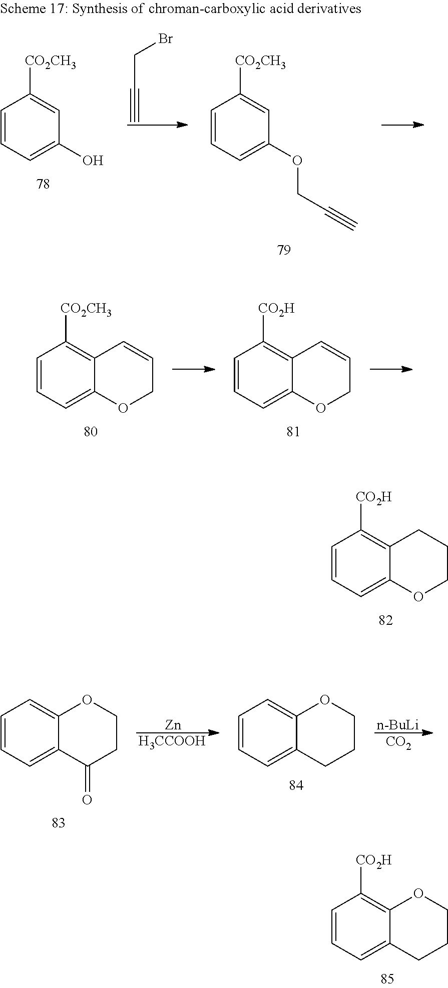

The synthesis of chroman-5-carboxylic acid derivatives can be started with the alkylation of 3-hydroxy-benzoic acid methyl ester (78; commercially available) with propargyl bromide in the presence of K₂CO₃ to give phenylether (79) which can be cyclised to the chromen derivative (80) by heating to reflux in N,N-diethylaniline. The carboxylic ester may be saponified (e.g. NaOH, MeOH/water) and the obtained chromen derivative (81) can be hydrogenated to give the desired acid (82). The corresponding chroman-8-carboxylic acid derivatives may be synthesized by reduction of 4-chromanone (83; commercially available) with zinc in acetic acid and subsequent ortho-metalation of the intermediate chroman derivative (84) with n-BuLi and trapping with carbon dioxide to give the desired acid (85).

Whenever the compounds of formula (I) are obtained in the form of mixtures of enantiomers, the enantiomers can be separated using methods known to the one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in presence or absence of an amine such as TEA, diethylamine) and eluent B (hexane), at a flow rate of 0.8 to 150 mL/min.

### Experimental Section

Abbreviations (as used herein and in the description above):
aq. aqueous
Ac Acetyl (such as in OAc=acetate, AcOH=acetic acid)
Boc tert-Butoxycarbonyl
BSA Bovine serum albumine
CHO Chinese hamster ovary
conc. Concentrated

### d Day(s)

DBU 1,8-Diazabicyclo[5.4.0]undec-7-ene

### DCM Dichloromethane

DDQ 2,3-Dichloro-5,6-dicyano-1,4-benzoquinone

### DIPEA Diisopropylethylamine

### DME Dimethoxyethane

### DMF N,N-Dimethylformamide

dppf diphenylphosphinoferrocene

### eq Equivalent(s)

ES Electron spray

### Et Ethyl

ether diethyl ether
EtOAc Ethyl acetate
FC flash chromatography on silica gel
FCS Foatal calf serum
FLIPR Fluorescent imaging plate reader

### h Hour(s)

HATU (O-(7-azabenzotriazol-1-yl)-1,1,3,3-tetramethyl-uronium hexafluorphoshate
HBSS Hank's balanced salt solution
HEPES 4-(2-hydroxyethyl)-piperazine-1-ethanesulfonic acid
HPLC High performance liquid chromatography
iPrOH isopropanol
KOtBu Potassium tert. butoxide
LC Liquid chromatography
M Exact mass (as used for LC-MS)

### Me Methyl

### MeCN Acetonitrile

mCPBA meta-chloroperoxybenzoic acid

### MeOH Methanol

### min Minute(s)

MS Mass spectroscopy

### N Normality

n-BuLi n-Butyl lithium

### NBS N-bromosuccinimide

### NMP N-methylpyrrolidone

### Ph Phenyl

### PPh₃ Triphenylphosphine

prep. Preparative
PPTS Pyridinium 4-toluenesulfonate
PTSA p-Toluenesulfonic acid
rt Room temperature

### sat Saturated

t_{R} Retention time
TBTU O-(Benzotriazol-1-yl)-N,N,N′,N′-tetramethyluronium tetrafluoroborate

### TEA Triethylamine

TFA trifluoroacetic acid

### Tf Trifluoromethansulfonyl

### THF Tetrahydrofuran

### TMS Trimethylsilyl

### I—Chemistry

All temperatures are stated in ° C. Compounds are characterized by ¹H-NMR (300 MHz: Varian Oxford or 400 MHz: Bruker Avance); chemical shifts are given in ppm relative to the solvent used; multiplicities: s=singlet, d=doublet, t=triplet; p=pentuplet, hex=hexet, hept=heptet, m=multiplet, br=broad, coupling constants are given in Hz); by LC-MS (Finnigan Navigator with HP 1100 Binary Pump and DAD, column: 4.6×50 mm, Zorbax SB-AQ, 5 μm, 120 Å, using two conditions: basic: eluent A: MeCN, eluent B: conc. NH₃ in water (1.0 mL/L), 5% to 95% CH₃CN; acidic: eluent A: MeCN, eluent B: TFA in water (0.4 mL/L), 5% to 95% CH₃CN), t_{R} is given in min; by TLC (TLC-plates from Merck, Silica gel 60 F₂₅₄); or by melting point. Compounds are purified by flash column chromatography on silica gel (FC) or by preparative HPLC (column: X-terra RP18, 50×19 mm, 5 μm, gradient: 10-95% MeCN in water containing 0.5% of formic acid).

The following examples illustrate the preparation of compounds of the invention but do not at all limit the scope thereof.

### Preparation of Precursors and Intermediates

### A.1 Synthesis of thiazole-4-carboxylic acid derivatives

### A.1.1 Synthesis of 3-chloro-2-oxo-propionic ester derivatives (general procedure)

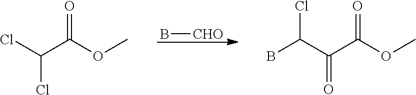

A solution of the respective benzaldehyde derivative B—CHO (338 mmol, 1.0 eq) and methyl dichloroacetate (338 mmol, 1.0 eq) in THF (100 mL) is added dropwise to a cold (−60° C.) suspension of KOtBu (335 mmol, 1.0 eq) in THF (420 mL). After 4 h the mixture is allowed to reach rt, stirred over night and concentrated in vacuo. DCM and ice-cold water are added, the layers are separated and the aq. layer is extracted twice with DCM. The combined organic layers are washed with ice-cold water and brine, dried over MgSO₄ and concentrated in vacuo to give the corresponding 3-chloro-2-oxo-propionic acid methyl ester derivative which is used without further purification.

### 3-Chloro-2-oxo-3-phenyl-propionic acid methyl ester

prepared by reaction of benzaldehyde with methyl dichloroacetate.

### 3-Chloro-2-oxo-3-m-tolyl-propionic acid methyl ester

prepared by reaction of 3-methyl-benzaldehyde with methyl dichloroacetate.

### 3-Chloro-2-oxo-3-p-tolyl-propionic acid methyl ester

prepared by reaction of 4-methyl-benzaldehyde with methyl dichloroacetate.

### 3-Chloro-3-(3-fluoro-phenyl)-2-oxo-propionic acid methyl ester

prepared by reaction of 3-fluoro-benzaldehyde with methyl dichloroacetate.

### 3-Chloro-3-(3,4-dichloro-phenyl)-2-oxo-propionic acid methyl ester

prepared by reaction of 3,4-dichloro-benzaldehyde with methyl dichloroacetate.

### 3-Chloro-3-(3,4-difluoro-phenyl)-2-oxo-propionic acid methyl ester

prepared by reaction of 3,4-difluoro-benzaldehyde with methyl dichloroacetate.

### 3-Chloro-3-(3,4-dimethyl-phenyl)-2-oxo-propionic acid methyl ester

prepared by reaction of 3,4-dimethyl-benzaldehyde with methyl dichloroacetate.

### 3-Chloro-3-(2-fluoro-phenyl)-2-oxo-propionic acid methyl ester

prepared by reaction of 2-fluoro-benzaldehyde with methyl dichloroacetate.

### 3-Chloro-3-(4-methoxy-phenyl)-2-oxo-propionic acid methyl ester

prepared by reaction of 4-methoxy-benzaldehyde with methyl dichloroacetate.

### 3-Chloro-3-(3-methoxy-phenyl)-2-oxo-propionic acid methyl ester

prepared by reaction of 3-methoxy-benzaldehyde with methyl dichloroacetate.

### 3-Chloro-3-(2-methoxy-phenyl)-2-oxo-propionic acid methyl ester

prepared by reaction of 2-methoxy-benzaldehyde with methyl dichloroacetate.

### 3-Chloro-3-(4-fluoro-phenyl)-2-oxo-propionic acid methyl ester

prepared by reaction of 4-fluoro-benzaldehyde with methyl dichloroacetate.

### 3-Chloro-2-oxo-3-(3-trifluoromethyl-phenyl)-propionic acid methyl ester

prepared by reaction of 3-trifluoromethyl-benzaldehyde with methyl dichloro-acetate.

### 3-Chloro-2-oxo-3-(4-trifluoromethyl-phenyl)-propionic acid methyl ester

prepared by reaction of 4-trifluoromethyl-benzaldehyde with methyl dichloroacetate.

### 3-chloro-3-(2,3-difluoro-phenyl)-2-oxo-propionic acid methyl ester

prepared by reaction of 2,3-difluoro-benzaldehyde with methyl dichloroacetate.

### 3-chloro-3-(3-fluoro-4-methyl-phenyl)-2-oxo-propionic acid methyl ester

prepared by reaction of 3-fluoro-4-methyl-benzaldehyde with methyl dichloroacetate

### 3-chloro-3-(2,3-difluoro-4-methyl-phenyl)-2-oxo-propionic acid methyl ester

prepared by reaction of 2,3-difluoro-4-methyl-benzaldehyde with methyl dichloroacetate

### A.1.2 Synthesis of thiazole-4-carboxylic acid methyl ester derivatives (general procedure)

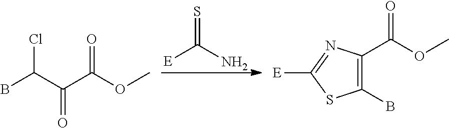

A solution of the respective thioacetamide (132 mmol, 1.0 eq) in MeCN (250 mL) is added to a mixture of the respective 3-chloro-2-oxo-propionic acid methyl ester derivative (132 mmol, 1.0 eq) and molecular sieves (4 Å, 12 g) in MeCN (60 mL). After stirring for 5 h the mixture is cooled in an ice-bath and the obtained precipitate is filtered off. The residue is washed with cold MeCN, dried, dissolved in MeOH (280 mL) and stirred at 50° C. for 6 h. The solvents are removed in vacuo to give the corresponding thiazole-4-carboxylic acid methyl ester derivatives.

### 2-Methyl-5-m-tolyl-thiazole-4-carboxylic acid methyl ester

prepared by reaction of 3-chloro-2-oxo-3-m-tolyl-propionic acid methyl ester with thioacetamide. LC-MS: t_{R}=0.94 min; [M+H]⁺=248.0.

### 2-Methyl-5-p-tolyl-thiazole-4-carboxylic acid methyl ester

prepared by reaction of 3-chloro-2-oxo-3-p-tolyl-propionic acid methyl ester with thioacetamide. LC-MS: t_{R}=0.93 min; [M+H]⁺=248.02.

### 5-(3-Fluoro-phenyl)-2-methyl-thiazole-4-carboxylic acid methyl ester

prepared by reaction of 3-chloro-3-(3-fluoro-phenyl)-2-oxo-propionic acid methyl ester with thioacetamide. LC-MS: t_{R}=0.91 min; [M+H]⁺=252.1.

### 5-(4-Fluoro-phenyl)-2-methyl-thiazole-4-carboxylic acid methyl ester

prepared by reaction of 3-chloro-3-(4-fluoro-phenyl)-2-oxo-propionic acid methyl ester with thioacetamide. ¹H-NMR (CDCl₃): δ=2.75 (s, 3H); 3.84 (s, 3H); 7.10 (m, 2H); 7.47 (m, 2H).

### 5-(2-Fluoro-phenyl)-2-methyl-thiazole-4-carboxylic acid methyl ester

prepared by reaction of 3-chloro-3-(2-fluoro-phenyl)-2-oxo-propionic acid methyl ester with thioacetamide. LC-MS: t_{R}=0.90 min; [M+H]⁺=251.99.

### 2-Methyl-5-(3-trifluoromethyl-phenyl)-thiazole-4-carboxylic acid methyl ester

prepared by reaction of 3-chloro-3-(3-trifluoromethyl-phenyl)-2-oxo-propionic acid methyl ester with thioacetamide. LC-MS: t_{R}=0.99 min; [M+H]⁺=301.99.

### 2-Methyl-5-(4-trifluoromethyl-phenyl)-thiazole-4-carboxylic acid methyl ester

prepared by reaction of 3-chloro-3-(4-trifluoromethyl-phenyl)-2-oxo-propionic acid methyl ester with thioacetamide. LC-MS: t_{R}=0.99 min; [M+H]⁺=301.99

### 2-Methyl-5-(3,4-dimethyl-phenyl)-thiazole-4-carboxylic acid methyl ester

prepared by reaction of 3-chloro-3-(3,4-dimethyl-phenyl)-2-oxo-propionic acid methyl ester with thioacetamide. LC-MS: t_{R}=0.96 min; [M+H]⁺=262.34.

### 2-Methyl-5-(3,4-dichloro-phenyl)-thiazole-4-carboxylic acid methyl ester

prepared by reaction of 3-chloro-3-(3,4-dichloro-phenyl)-2-oxo-propionic acid methyl ester with thioacetamide. LC-MS: t_{R}=0.99 min; [M+H]⁺=302.22.

### 2-Methyl-5-(3,4-difluoro-phenyl)-thiazole-4-carboxylic acid methyl ester

prepared by reaction of 3-chloro-3-(3,4-difluoro-phenyl)-2-oxo-propionic acid methyl ester with thioacetamide. LC-MS: t_{R}=0.92 min; [M+H]⁺=270.29.

### 5-(4-Methoxy-phenyl)-2-methyl-thiazole-4-carboxylic acid methyl ester

prepared by reaction of 3-chloro-3-(4-methoxy-phenyl)-2-oxo-propionic acid methyl ester with thioacetamide. LC-MS: t_{R}=0.90 min; [M+H]⁺=263.93.

### 5-(3-Methoxy-phenyl)-2-methyl-thiazole-4-carboxylic acid methyl ester

prepared by reaction of 3-chloro-3-(3-methoxy-phenyl)-2-oxo-propionic acid methyl ester with thioacetamide. LC-MS: t_{R}=0.90 min; [M+H]⁺=263.87.

### 5-(2-Methoxy-phenyl)-2-methyl-thiazole-4-carboxylic acid methyl ester

prepared by reaction of 3-chloro-3-(2-methoxy-phenyl)-2-oxo-propionic acid methyl ester with thioacetamide. LC-MS: t_{R}=0.88 min; [M+H]⁺=264.05.

### 5-Phenyl-2-methyl-thiazole-4-carboxylic acid methyl ester

prepared by reaction of 3-chloro-3-phenyl-2-oxo-propionic acid methyl ester with thioacetamide. LC-MS: t_{R}=0.88 min; [M+H]⁺=234.23.

### 2-Methyl-5-(2,3-difluoro-phenyl)-thiazole-4-carboxylic acid methyl ester

prepared by reaction of 3-chloro-3-(2,3-difluoro-phenyl)-2-oxo-propionic acid methyl ester with thioacetamide. LC-MS: t_{R}=0.82 min; [M+H]⁺=270.29

### 5-(3-Fluoro-4-methyl-phenyl)-2-methyl-thiazole-4-carboxylic acid methyl ester

prepared by reaction of 3-chloro-3-(3-fluoro-4-methyl-phenyl)-2-oxo-propionic acid methyl ester with thioacetamide. LC-MS: t_{R}=1.00 min; [M+H]⁺=266.01

### 5-(2,3-Difluoro-4-methyl-phenyl)-2-methyl-thiazole-4-carboxylic acid methyl ester

prepared by reaction of 3-chloro-3-(2,3-difluoro-4-methyl-phenyl)-2-oxo-propionic acid methyl ester with thioacetamide. LC-MS: t_{R}=0.95 min; [M+H]⁺=284.30

### 2-Amino-5-m-tolyl-thiazole-4-carboxylic acid methyl ester

prepared by reaction of 3-chloro-2-oxo-3-m-tolyl-propionic acid methyl ester with thiourea LC-MS: t_{R}=0.83 min; [M+H]⁺=249.07

### 2-Cyclopropyl-5-(3-fluoro-phenyl)-thiazole-4-carboxylic acid methyl ester

prepared by reaction of 3-chloro-3-(3-fluoro-phenyl)-2-oxo-propionic acid methyl ester with cyclopropanecarbothioic acid amide (Boys M. et al, Synth. Commun. 2006, 36, 3, 295-298) LC-MS: t_{R}=1.02 min; [M+H]⁺=278.04.

### 2-Amino-5-(3-methoxy-phenyl)-thiazole-4-carboxylic acid methyl ester

prepared by reaction of 3-chloro-3-(3-methoxy-phenyl)-2-oxo-propionic acid methyl ester with thiourea LC-MS: t_{R}=0.75 min; [M+H]⁺=265.25.

### 2-Amino-5-(3-fluoro-phenyl)-thiazole-4-carboxylic acid methyl ester

prepared by reaction of 3-chloro-3-(3-fluoro-phenyl)-2-oxo-propionic acid methyl ester with thiourea LC-MS: t_{R}=0.75 min; [M+H]⁺=253.17.

### 2-Amino-5-(4-fluoro-phenyl)-thiazole-4-carboxylic acid methyl ester

prepared by reaction of 3-chloro-3-(4-fluoro-phenyl)-2-oxo-propionic acid methyl ester with thiourea LC-MS: t_{R}=0.82 min; [M+H]⁺=253.03.

### 2-Amino-5-(3,4-dimethyl-phenyl)-thiazole-4-carboxylic acid methyl ester

prepared by reaction of 3-chloro-3-(3,4-dimethyl-phenyl)-2-oxo-propionic acid methyl ester with thiourea LC-MS: t_{R}=0.85 min; [M+H]⁺=263.00.

### A.1.3 Synthesis of thiazole-4-carboxylic acid derivatives (general procedure)

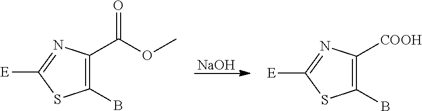

A solution of the respective thiazole-4-carboxylic acid methyl ester (96.2 mmol) in a mixture of THF (150 mL) and MeOH (50 mL) is treated with 1M aq. NaOH (192 mL). After stirring for 3 h a white suspension is formed and the organic volatiles are removed in vacuo. The remaining mixture is diluted with water (100 mL), cooled in an ice-bath and acidified (pH=3-4) by addition of 1M aq. HCl. The suspension is filtered and the residue is washed with cold water. After drying the corresponding thiazole-4-carboxylic acid derivative is obtained.

### 2-Methyl-5-m-tolyl-thiazole-4-carboxylic acid

prepared by saponification of 2-methyl-5-m-tolyl-thiazole-4-carboxylic acid methyl ester. LC-MS: t_{R}=0.83 min; [M+H]⁺=233.99.

### 2-Methyl-5-p-tolyl-thiazole-4-carboxylic acid

prepared by saponification of 2-methyl-5-p-tolyl-thiazole-4-carboxylic acid methyl ester. LC-MS: t_{R}=0.83 min; [M+H]⁺=234.0.

### 5-(3-Fluoro-phenyl)-2-methyl-thiazole-4-carboxylic acid

prepared by saponification of 5-(3-fluoro-phenyl)-2-methyl-thiazole-4-carboxylic acid methyl ester. LC-MS: t_{R}=0.82 min; [M+H]⁺=238.1.

### 5-(4-Fluoro-phenyl)-2-methyl-thiazole-4-carboxylic acid

prepared by saponification of 5-(4-fluoro-phenyl)-2-methyl-thiazole-4-carboxylic acid methyl ester. ¹H-NMR (DMSO-d₆): δ=2.67 (s, 3H); 7.27 (m, 2H); 7.53 (m, 2H); 12.89 (br.s, 1H).

### 2-Methyl-5-(3-trifluoromethyl-phenyl)-thiazole-4-carboxylic acid

prepared by saponification of 2-methyl-5-(3-trifluoromethyl-phenyl)-thiazole-4-carboxylic acid methyl ester. LC-MS: t_{R}=0.88 min; [M+H]⁺=287.99.

### 2-Methyl-5-(4-trifluoromethyl-phenyl)-thiazole-4-carboxylic acid

prepared by saponification of 2-methyl-5-(4-trifluoromethyl-phenyl)-thiazole-4-carboxylic acid methyl ester. LC-MS: t_{R}=0.90 min; [M+H]⁺=287.99.

### 2-Methyl-5-(3,4-dimethyl-phenyl)-thiazole-4-carboxylic acid

prepared by saponification of 2-methyl-5-(3,4-dimethyl-phenyl)-thiazole-4-carboxylic acid methyl ester. LC-MS: t_{R}=0.97 min; [M+H]⁺=382.38.

### 2-Methyl-5-(3,4-dichloro-phenyl)-thiazole-4-carboxylic acid

prepared by saponification of 2-methyl-5-(3,4-dichloro-phenyl)-thiazole-4-carboxylic acid methyl ester. LC-MS: t_{R}=0.88 min; [M+H]⁺=288.22.

### 2-Methyl-5-(3,4-difluoro-phenyl)-thiazole-4-carboxylic acid

prepared by saponification of 2-methyl-5-(3,4-difluoro-phenyl)-thiazole-4-carboxylic acid methyl ester. LC-MS: t_{R}=0.82 min; [M+H]⁺=256.25.

### 2-Methyl-5-(2-methoxy-phenyl)-thiazole-4-carboxylic acid

prepared by saponification of 2-methyl-5-(2-methoxy-phenyl)-thiazole-4-carboxylic acid methyl ester. LC-MS: t_{R}=0.78 min; [M+H]⁺=249.98.

### 2-Methyl-5-(3-methoxy-phenyl)-thiazole-4-carboxylic acid

prepared by saponification of 2-methyl-5-(3-methoxy-phenyl)-thiazole-4-carboxylic acid methyl ester. LC-MS: t_{R}=0.80 min; [M+H]⁺=250.04.

### 2-Methyl-5-(4-methoxy-phenyl)-thiazole-4-carboxylic acid

prepared by saponification of 2-methyl-5-(4-methoxy-phenyl)-thiazole-4-carboxylic acid methyl ester. LC-MS: t_{R}=0.80 min; [M+H]⁺=250.04.

### 2-Methyl-5-phenyl-thiazole-4-carboxylic acid

prepared by saponification of 2-methyl-5-phenyl-thiazole-4-carboxylic acid methyl ester. LC-MS: t_{R}=0.78 min; [M+H]⁺=220.01.

### 2-Methyl-5-(2,3-difluoro-phenyl)-thiazole-4-carboxylic acid

prepared by saponification of 2-methyl-5-(2,3-difluoro-phenyl)-thiazole-4-carboxylic acid methyl ester. LC-MS: t_{R}=0.82 min; [M+H]⁺=256.25

### 5-(3-Fluoro-4-methyl-phenyl)-2-methyl-thiazole-4-carboxylic acid

prepared by saponification of 5-(3-fluoro-4-methyl-phenyl)-2-methyl-thiazole-4-carboxylic acid methyl ester. LC-MS: t_{R}=0.89 min; [M+H]⁺=251.98

### 5-(2,3-Difluoro-4-methyl-phenyl)-2-methyl-thiazole-4-carboxylic acid

prepared by saponification of 5-(2,3-difluoro-4-methyl-phenyl)-2-methyl-thiazole-4-carboxylic acid methyl ester. LC-MS: t_{R}=0.84 min; [M+H]⁺=270.35

### 2-Cyclopropyl-5-(3-fluoro-phenyl)-thiazole-4-carboxylic acid methyl ester

prepared by saponification of 2-cyclopropyl-5-(3-fluoro-phenyl)-thiazole-4-carboxylic acid methyl ester. LC-MS: t_{R}=0.92 min; [M+H]⁺=264.01

### A.1.4 Synthesis of 2-dimethylamino-thiazole-4-carboxylic acid derivatives

### A.1.4.1 2-Dimethylamino-5-m-tolyl-thiazole-4-carboxylic acid

### A.1.4.1.1 2-Bromo-5-m-tolyl-thiazole-4-carboxylic acid methyl ester

To a cold (0-5° C.) solution of CuBr₂ (43.5 g) in MeCN (700 mL) was added dropwise over 15 min. isoamylnitrite (41 mL) and then portionwise over 45 min. 2-amino-5-m-tolyl-thiazole-4-carboxylic acid methyl ester (47.41 g). After stirring at 0° C. for 15 min., the reaction mixture was heated at 65° C. for 2 h. After cooling to rt, the reaction mixture was concentrated in vacuo to yield a crude dark-brown solid.

FC (EtOAc/n-heptane: 1/4) gave 40.18 g (62%) of the title compound as a yellow oil. LC-MS: t_{R}=1.05 min; [M+H]⁺=313.96.

### A.1.4.1.2 2-Dimethylamino-5-m-tolyl-thiazole-4-carboxylic acid methyl ester

A mixture of 2-bromo-5-m-tolyl-thiazole-4-carboxylic acid methyl ester (2 g), dimethylamine (40% in water) (25.75 mL) in MeCN (30 mL) was stirred at rt for 20 h. Then water (30 mL) was added and the pH was adjusted to pH 3 with 10% citric acid. The reaction mixture was extracted with EtOAc (3×), the combined organic extracts were dried (MgSO₄), filtered and concentrated to yield the title compound as a yellow oil (2 g) which was used for the next step without further purification.

LC-MS: t_{R}=0.98 min; [M+H]⁺=277.03.

### A.1.4.1.3 2-Dimethylamino-5-m-tolyl-thiazole-4-carboxylic acid

A solution of 2-dimethylamino-5-m-tolyl-thiazole-4-carboxylic acid methyl ester (2 g) in a mixture of THF (9.5 mL) and MeOH (7 mL) is treated with 1M aq. NaOH (14 mL). After stirring for 3 h a white suspension is formed and the organic volatiles are removed in vacuo. The remaining mixture is diluted with water (10 mL), cooled in an ice-bath and acidified (pH=3-4) by addition of 1M aq. HCl. The suspension is filtered and the residue is washed with cold water. After drying the title compound is obtained (1 g, 52%).

LC-MS: t_{R}=0.85 min; [M+H]⁺=263.06

The following compounds have been prepared in analogy:

### A.1.4.2 2-Dimethylamino-5-(3-methoxy-phenyl)-tolyl-thiazole-4-carboxylic acid

### A.1.4.2.1 2-Bromo-5-(3-methoxy-phenyl)-thiazole-4-carboxylic acid methyl ester

prepared by reaction of 2-amino-5-(3-methoxy-phenyl)-thiazole-4-carboxylic acid methyl ester according to procedure A.1.4.1.1, LC-MS: t_{R}=0.97 min; [M+H]⁺=330.20

### A.1.4.2.2 2-Dimethylamino-5-(3-methoxy-phenyl)-thiazole-4-carboxylic acid methyl ester

prepared by reaction of 2-bromo-5-(3-methoxy-phenyl)-thiazole-4-carboxylic acid methyl ester according to procedure A.1.4.1.2, LC-MS: t_{R}=0.97 min; [M+H]⁺=330.20

### A.1.4.2.3 2-Dimethylamino-5-(3-methoxy-phenyl)-thiazole-4-carboxylic acid

prepared by reaction of 2-dimethylamino-5-(3-methoxy-phenyl)-thiazole-4-carboxylic acid according to procedure A.1.4.1.3, LC-MS: t_{R}=0.97 min; [M+H]⁺=330.20

### A.1.4.3 2-Dimethylamino-5-(3-fluoro-phenyl)-thiazole-4-carboxylic acid

### A.1.4.3.1 2-Bromo-5-(3-fluoro-phenyl)-thiazole-4-carboxylic acid methyl ester

prepared by reaction of 2-amino-5-(3-fluoro-phenyl)-thiazole-4-carboxylic acid methyl ester according to procedure A.1.4.1.1, LC-MS: t_{R}=0.95 min; [M+H]⁺=316.09.

### A.1.4.3.2 2-Dimethylamino-5-(3-fluoro-phenyl)-thiazole-4-carboxylic acid methyl ester

prepared by reaction of 2-bromo-5-(3-fluoro-phenyl)-thiazole-4-carboxylic acid methyl ester according to procedure A.1.4.1.2, LC-MS: t_{R}=0.98 min; [M+H]⁺=281.31.

### A.1.4.3.3 2-Dimethylamino-5-(3-fluoro-phenyl)-thiazole-4-carboxylic acid

prepared by reaction of 2-dimethylamino-5-(3-fluoro-phenyl)-thiazole-4-carboxylic acid according to procedure A.1.4.1.3, LC-MS: t_{R}=0.85 min; [M+H]⁺=267.26.

### A.1.4.4 2-Dimethylamino-5-(4-fluoro-phenyl)-thiazole-4-carboxylic acid

### A.1.4.4.1 2-Bromo-5-(4-fluoro-phenyl)-thiazole-4-carboxylic acid methyl ester

prepared by reaction of 2-amino-5-(4-fluoro-phenyl)-thiazole-4-carboxylic acid methyl ester according to procedure A.1.4.1.1, LC-MS: t_{R}=0.97 min; [M+H]⁺=316.09.

### A.1.4.4.2 2-Dimethylamino-5-(4-fluoro-phenyl)-thiazole-4-carboxylic acid methyl ester

prepared by reaction of 2-bromo-5-(3,4-dimethyl-phenyl)-thiazole-4-carboxylic acid methyl ester according to procedure A.1.4.1.2, LC-MS: t_{R}=0.97 min; [M+H]⁺=281.33.

### A.1.4.4.3 2-Dimethylamino-5-(4-fluoro-phenyl)-thiazole-4-carboxylic acid

prepared by reaction of 2-dimethylamino-5-(3,4-dimethyl-phenyl)-thiazole-4-carboxylic acid according to procedure A.1.4.1.3, LC-MS: t_{R}=0.83 min; [M+H]⁺=267.27.

### A.1.4.5 2-Dimethylamino-5-(3,4-dimethyl-phenyl)-thiazole-4-carboxylic acid

### A.1.4.5. 1 2-Bromo-5-(3,4-dimethyl-phenyl)-thiazole-4-carboxylic acid methyl ester

prepared by reaction of 2-amino-5-(3,4-dimethyl-phenyl)-thiazole-4-carboxylic acid methyl ester according to procedure A.1.4.1.1, LC-MS: t_{R}=1.05 min; [M+H]⁺=326.2.

### A.1.4.5.2 2-Dimethylamino-5-(3,4-dimethyl-phenyl)-thiazole-4-carboxylic acid methyl ester

prepared by reaction of 2-bromo-5-(3,4-dimethyl-phenyl)-thiazole-4-carboxylic acid methyl ester according to procedure A.1.4.1.2, LC-MS: t_{R}=1.01 min; [M+H]⁺=291.39.

### A.1.4.5.3 2-Dimethylamino-5-(3,4-dimethyl-phenyl)-thiazole-4-carboxylic acid

prepared by reaction of 2-dimethylamino-5-(3,4-dimethyl-phenyl)-thiazole-4-carboxylic acid according to procedure A.1.4.1.3, LC-MS: t_{R}=0.89 min; [M+H]⁺=277.30.

### A.1.5 Synthesis of 5-(3-fluoro-phenyl)-thiazole-4-carboxylic acid

### A.1.5.1 5-(3-Fluoro-phenyl)-thiazole-4-carboxylic acid methyl ester

A mixture of 2-bromo-5-m-tolyl-thiazole-4-carboxylic acid methyl ester (2 g), Pd—C 10% (4 g) in EtOH (80 mL) was hydrogenated at rt and atmospheric pressure for 20 h. The reaction mixture was then filtered over celite and the filtrate was concentrated to yield the title compound (1.6 g, 100%). LC-MS: t_{R}=0.92 min; [M+H]⁺=238.06

### A.1.5.2 5-(3-Fluoro-phenyl)-thiazole-4-carboxylic acid

A solution of 5-(3-fluoro-phenyl)-thiazole-4-carboxylic acid methyl ester (1.6 g) in a mixture of THF (12 mL) and MeOH (6 mL) is treated with 1M aq. NaOH (12 mL). After stirring for 3 h a white suspension is formed and the organic volatiles are removed in vacuo. The remaining mixture is diluted with water (10 mL), cooled in an ice-bath and acidified (pH=3-4) by addition of 1M aq. HCl. The suspension is filtered and the residue is washed with cold water. After drying the title compound is obtained (0.87 g, 58%). LC-MS: t_{R}=0.80 min; [M+H]⁺=224.04

### A.2 Synthesis of 2-methyl-oxazole-4-carboxylic acid derivatives

### A.2.1 Synthesis of 2-acetylamino-3-oxo-propionic acid methyl ester derivatives (general procedure)

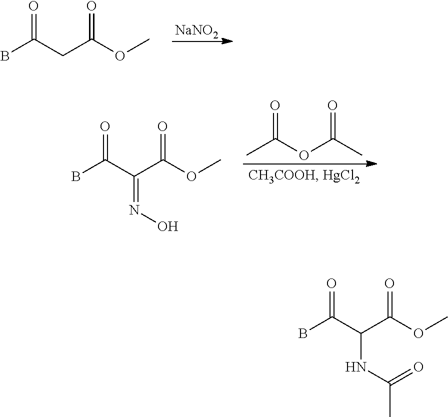

A solution of the respective 3-oxo-propionic acid methyl ester derivative (4.8 mmol, 1.0 eq.) in glacial acetic acid (1.9 mL) was cooled to 10° C. and at this temperature was added a solution of NaNO₂ (5.6 mmol, 1.16 eq.) in water (0.68 mL). After the addition was complete (15 min), the solution was allowed to warm to room temperature and stirred for 2 h. Then the solution was poured into water (10 mL) and after a few minutes crystals begun to appear. This suspension was cooled in an ice-bath and crystals were collected by filtration. The cake was washed several times with cold water and the water was removed by the azeotrope of toluene-water in vacuo to give 2-hydroxyimino-3-oxo-propionic acid methyl ester derivatives which were dissolved in a mixture of acetic anhydride (1.375 mL) and glacial acetic acid (1.8 mL). To this solution was added sodium acetate (0.296 mmol, 0.06 eq.) and HgCl₂ (0.01 mmol, 0.002 eq.). The mixture was refluxed for 1 h, then cooled to room temperature and filtered. The solid was rinsed with ether, the organic filtrate was recovered, washed 3 times with water and one time with 1M aq. K₂CO₃. The organic layer was dried over MgSO₄, filtered and concentrated. The crude products were purified by FC to afford the corresponding 2-acetylamino-3-oxo-propionic acid methyl ester derivatives.

### 2-Acetylamino-3-oxo-3-(3-trifluoromethyl-phenyl)-propionic acid methyl ester

prepared according to general procedure A.2.1 from 3-oxo-(3-trifluoromethyl-phenyl)-propionic acid methyl ester.

### 2-Acetylamino-3-oxo-3-m-tolyl-propionic acid methyl ester

prepared according to general procedure A.2.1 from 3-oxo-3-m-tolyl-propionic acid methyl ester.

### 2-Acetylamino-3-oxo-3-p-tolyl-propionic acid methyl ester

prepared according to general procedure A.2.1 from 3-oxo-3-p-tolyl-propionic acid methyl ester.

### 2-Acetylamino-3-(4-fluoro-phenyl)-3-oxo-propionic acid methyl ester

prepared according to general procedure A.2.1 from 3-oxo-3-(4-fluoro-phenyl)-propionic acid methyl ester.

### 2-Acetylamino-3-(4-methoxy-phenyl)-3-oxo-propionic acid methyl ester

prepared according to general procedure A.2.1 from 3-oxo-3-(4-methoxy-phenyl)-propionic acid methyl ester.

### 2-Acetylamino-3-(3-fluoro-phenyl)-3-oxo-propionic acid methyl ester

prepared according to general procedure A.2.1 from 3-oxo-3-(3-fluoro-phenyl)-propionic acid methyl ester.

### 2-Acetylamino-3-(3-chloro-phenyl)-3-oxo-propionic acid methyl ester

prepared according to general procedure A.2.1 from 3-oxo-3-(3-chloro-phenyl)-propionic acid methyl ester.

### 2-Acetylamino-3-(3-trifluoromethoxy-phenyl)-3-oxo-propionic acid methyl ester

prepared according to general procedure A.2.1 from 3-oxo-3-(3-trifluoromethoxy-phenyl)-propionic acid methyl ester.

### 2-Acetylamino-3-oxo-3-phenyl-propionic acid methyl ester

prepared according to general procedure A.2.1 from 3-oxo-3-phenyl-propionic acid methyl ester.

### A.2.2 Synthesis of 2-methyl-oxazole-4-carboxylic acid derivatives (general procedure)

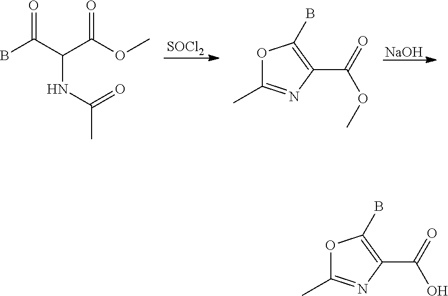

A solution of the respective 2-acetylamino-3-oxo-propionic acid methyl ester derivative (0.63 mmol, 1.0 eq.) in chloroform (0.4 mL) was cooled to 0° C. in an ice/NaCl bath. SOCl₂ (0.88 mmol, 1.4 eq.) was added to the stirred solution and the temperature was maintained at 0° C. for 30 minutes. Then the solution was stirred and refluxed for one hour. Another 0.25 eq. of SOCl₂ was added and the reaction mixture was refluxed for another hour.

The excess SOCl₂ was quenched with 1M aq. K₂CO₃. The aq. layer was extracted twice with ether. The combined organic phases were washed once with water and dried over MgSO₄, filtered and concentrated yielding the corresponding 2-methyl-oxazole-4-carboxylic acid methyl ester derivative. The respective 2-methyl-oxazole-4-carboxylic acid methyl ester derivative was dissolved in a mixture of EtOH (0.7 ml) and 2N aq. NaOH (0.7 mL, 2.5 eq.). The mixture was stirred at rt for 2 hours.

The reaction mixture was washed once with ether and this organic layer was discarded. The aq. layer was then acidified with conc. HCl and extracted twice with ether. Both organic layers were combined, dried over MgSO₄ and concentrated in vacuo to afford the corresponding 2-methyl-oxazole-4-carboxylic acid derivatives.

### 2-Methyl-5-m-tolyl-oxazole-4-carboxylic acid

prepared according to general procedure A.2.2 from 2-acetylamino-3-oxo-3-m-tolyl-propionic acid methyl ester LC-MS: t_{R}=0.51 min; [M−H]⁺=216.33.

### 2-Methyl-5-(3-trifluoromethyl-phenyl)-oxazole-4-carboxylic acid

prepared according to general procedure A.2.2 from 2-acetylamino-3-oxo-3-(3-trifluoromethyl-phenyl)-propionic acid methyl ester. LC-MS: t_{R}=0.55 min; [M−H]⁺=270.24.

### 2-Methyl-5-p-tolyl-oxazole-4-carboxylic acid

prepared according to general procedure A.2.2 from 2-acetylamino-3-oxo-3-p-tolyl-propionic acid methyl ester. LC-MS: t_{R}=0.55 min; [M−H]⁺=216.34.

### 5-(4-Fluoro-phenyl)-2-methyl-oxazole-4-carboxylic acid

prepared according to general procedure A.2.2 from 2-acetylamino-3-(4-fluoro-phenyl)-3-oxo-propionic acid methyl ester. LC-MS: t_{R}=0.49 min; [M−H]⁺=220.30.

### 5-(4-Methoxy-phenyl)-2-methyl-oxazole-4-carboxylic acid

prepared according to general procedure A.2.2 from 2-acetylamino-3-(4-methoxy-phenyl)-3-oxo-propionic acid methyl ester. LC-MS: t_{R}=0.77 min; [M+H]⁺=234.31.

### 5-(3-Methoxy-phenyl)-2-methyl-oxazole-4-carboxylic acid

prepared according to general procedure A.2.2 from 2-acetylamino-3-(3-methoxy-phenyl)-3-oxo-propionic acid methyl ester. LC-MS: t_{R}=0.49 min; [M+H]⁺=232.30.

### 5-(3-Fluoro-phenyl)-2-methyl-oxazole-4-carboxylic acid

prepared according to general procedure A.2.2 from 2-acetylamino-3-(3-fluoro-phenyl)-3-oxo-propionic acid methyl ester. LC-MS: t_{R}=0.49 min; [M+H]⁺=221.99.

### 5-(3-Chloro-phenyl)-2-methyl-oxazole-4-carboxylic acid

prepared according to general procedure A.2.2 from 2-acetylamino-3-(3-chloro-phenyl)-3-oxo-propionic acid methyl ester. LC-MS: t_{R}=0.53 min; [M+H]⁺=238.97.

### 5-(3-Trifluoromethoxy-phenyl)-2-methyl-oxazole-4-carboxylic acid

prepared according to general procedure A.2.2 from 2-acetylamino-3-(3-trifluoromethoxy-phenyl)-3-oxo-propionic acid methyl ester. LC-MS: t_{R}=0.93 min; [M+H]⁺=288.06.

### 2-Methyl-5-phenyl-oxazole-4-carboxylic acid

prepared according to general procedure A.2.2 from 2-acetylamino-3-oxo-3-phenyl-propionic acid methyl ester. LC-MS: t_{R}=0.80 min; [M+H]⁺=204.42.

### A.3 Biphenyl-2-carboxylic acid derivatives

The following biphenyl-2-carboxylic acid derivatives are commercially available:
Biphenyl-2-carboxylic acid;
4′-Methyl-biphenyl-2-carboxylic acid;
3′-Methyl-biphenyl-2-carboxylic acid;
3′,4′-Dimethyl-biphenyl-2-carboxylic acid;
4′-Methoxy-biphenyl-2-carboxylic acid;
3′-Methoxy-biphenyl-2-carboxylic acid;
4′-Fluoro-biphenyl-2-carboxylic acid.

### A.4 Synthesis of 2-m-tolyl-thiophene-3-carboxylic acid

A mixture of 2-bromo-3-thiophenecarboxylic acid (1 g), 3-tolyl-boronic acid (656.65 mg), Pd(PPh₃)₄ (162.5 mg), aq 2M K₂CO₃ (11.7 mL) in a mixture of iPrOH (10 mL) and toluene (10 mL) was stirred at 80° C. under nitrogen for 5 h. After cooling to rt, the reaction mixture was diluted with ether, washed with 2M NaOH. The aq. phase was acidified with 2N HCl until pH 1. The resulting white precipitate was filtered off, washed with cold water and dried in vacuo to yield the title compound (0.59 g, 58%) as a white solid. LC-MS: t_{R}=0.51 min; [M+H]⁺=218.99.

### A.5 Synthesis of thiazole-5-carboxylic acid derivatives

### A.5.1 Synthesis of 2-chloro-3-oxo-propionic acid ethyl ester derivatives (general procedure)

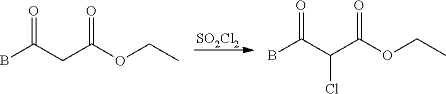

A mixture of 3-oxo-3-propionic acid ethyl ester derivative (5.5 mmol), sulfuryl chloride (5.5 mmol) in chloroform (3.3 mL) was stirred at reflux for 20 h. After cooling to rt, the reaction mixture was washed with water and the organic extract was concentrated in vacuo to yield the desired 2-chloro-3-oxo-propionic acid ethyl ester derivative which was used for the next step without further purification

### 2-Chloro-3-(3-methoxy-phenyl)-3-oxo-propionic acid ethyl ester

prepared by reaction with 3-(3-methoxy-phenyl)-3-oxo-propionic acid ethyl ester.

### 2-Chloro-3-(3-chloro-phenyl)-3-oxo-propionic acid ethyl ester

prepared by reaction with 3-(3-chloro-phenyl)-3-oxo-propionic acid ethyl ester.

### 2-Chloro-3-oxo-3-(3-trifluoromethyl-phenyl)-propionic acid ethyl ester

prepared by reaction with 3-oxo-3-(3-chloro-phenyl)-propionic acid ethyl ester.

### 2-Chloro-3-(4-fluoro-phenyl)-3-oxo-propionic acid ethyl ester

prepared by reaction with 3-(3-fluoro-phenyl)-3-oxo-propionic acid ethyl ester.

### 2-Chloro-3-oxo-3-p-tolyl-propionic acid ethyl ester

prepared by reaction with 3-oxo-3-p-tolyl-propionic acid ethyl ester.

### A.5.2 Synthesis of 2-methyl-thiazole-5-carboxylic acid methyl ester derivatives (general procedure)

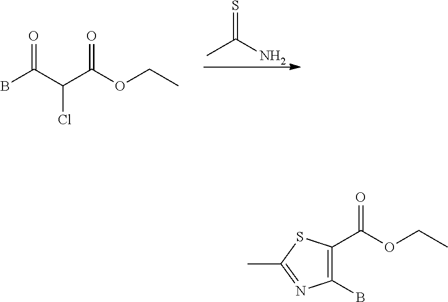

A mixture of the respective 2-chloro-3-oxo-propionic acid ethyl ester derivative (5.5 mmol), thioacetamide (6.75 mmol), NaHCO₃ (6 mmol) in dry THF (12 mL) was stirred at reflux for 5 h. After cooling to rt, the reaction mixture was concentrated in vacuo. The residue was purified by FC (EtOAc/heptane: 1/9 to 4/6) to give the desired 2-methyl-thiazole-5-carboxylic acid ethyl ester.

### 4-(3-Methoxy-phenyl)-2-methyl-thiazole-5-carboxylic acid ethyl ester

prepared by reaction of 2-chloro-3-(3-methoxy-phenyl)-3-oxo-propionic acid ethyl ester with thioacetamide. LC-MS: t_{R}=1.14 min; [M+H]⁺=278.14

### 4-(3-Chloro-phenyl)-2-methyl-thiazole-5-carboxylic acid ethyl ester

prepared by reaction of 2-chloro-3-(3-chloro-phenyl)-3-oxo-propionic acid ethyl ester with thioacetamide. LC-MS: t_{R}=0.89 min; [M+H]⁺=282.13

### 2-Methyl-4-(3-trifluoromethyl-phenyl)-thiazole-5-carboxylic acid ethyl ester

prepared by reaction of 2-chloro-3-oxo-3-(3-trifluoromethyl-phenyl)-propionic acid ethyl ester with thioacetamide. LC-MS: t_{R}=0.93 min; [M+H]⁺=316.16

### 4-(4-Fluoro-phenyl)-2-methyl-thiazole-5-carboxylic acid ethyl ester

prepared by reaction of 2-chloro-3-(4-fluoro-phenyl)-3-oxo-propionic acid ethyl ester with thioacetamide. LC-MS: t_{R}=0.95 min; [M+H]⁺=266.11

### 2-Methyl-4-p-tolyl-thiazole-5-carboxylic acid ethyl ester

prepared by reaction of 2-chloro-3oxo-3-p-tolyl-propionic acid ethyl ester with thioacetamide. LC-MS: t_{R}=1.01 min; [M+H]⁺=262.14

### A.5.3 Synthesis of 2-methyl-thiazole-5-carboxylic acid derivatives (general procedure)

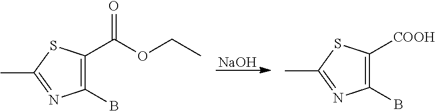

A solution of the respective 2-methyl-thiazole-5-carboxylic acid ethyl ester (5 mmol) in EtOH (2 mL) is treated with 2M aq. NaOH (2 mL). After stirring for 3 h a white suspension is formed and the organic volatiles are removed in vacuo. The remaining mixture is diluted with water (2 mL), cooled in an ice-bath and acidified (pH=3-4) by addition of 1M aq. HCl. The suspension is filtered and the residue is washed with cold water. After drying the corresponding 2-methyl-thiazole-5-carboxylic acid derivative is obtained.

### 4-(3-Methoxy-phenyl)-2-methyl-thiazole-5-carboxylic acid

prepared by saponification of 4-(3-Methoxy-phenyl)-2-methyl-thiazole-5-carboxylic acid ethyl ester. LC-MS: t_{R}=0.79 min; [M+H]⁺=250.28

### 4-(3-Chloro-phenyl)-2-methyl-thiazole-5-carboxylic acid

prepared by saponification of 4-(3-Chloro-phenyl)-2-methyl-thiazole-5-carboxylic acid ethyl ester. LC-MS: t_{R}=0.85 min; [M+H]⁺=253.98

### 2-Methyl-4-(3-trifluoromethyl-phenyl)-thiazole-5-carboxylic acid

prepared by reaction of 2-Methyl-4-(3-trifluoromethyl-phenyl)-thiazole-5-carboxylic acid ethyl ester. LC-MS: t_{R}=0.90 min; [M+H]⁺=288.99

### 4-(4-Fluoro-phenyl)-2-methyl-thiazole-5-carboxylic acid

prepared by reaction of 4-(4-Fluoro-phenyl)-2-methyl-thiazole-5-carboxylic acid ethyl ester

LC-MS: t_{R}=0.81 min; [M+H]⁺=237.99

### 2-Methyl-4-p-tolyl-thiazole-5-carboxylic acid

prepared by reaction of 2-Methyl-4-p-tolyl-thiazole-5-carboxylic acid ethyl ester. LC-MS: t_{R}=0.83 min; [M+H]⁺=234.02

### A.6 Synthesis of pyrazine-2-carboxylic acid derivatives

### A.6.1 Synthesis of pyrazine-2-carbonitrile derivatives

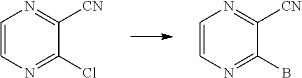

A mixture of the respective boronic acid derivative (B—B(OH)₂) (21.5 mmol), 3-chloro-pyrazine-2-carbonitrile (21.5 mmol), a solution of K₂CO₃ (59.4 mmol) in water (30 mL), PPh₃ (3. 2 mmol), Pd(OAc)₂ (1.05 mmol) in dry DME was stirred at reflux under inert atmosphere for 16 h. After cooling to rt, the reaction mixture was diluted with EtOAc, filtered over celite, the filtrate was dried over MgSO₄, filtered and concentrated in vacuo to yield the desired pyrazine-2-carbonitrile derivative which was used for the next step without further purification

### 3-m-Tolyl-pyrazine-2-carbonitrile

prepared by reaction with commercially available 3-m-tolyl-boronic acid LC-MS: t_{R}=0.88 min; [M+H+ MeCN]⁺=243.63

### 3-(3,4-Dimethyl-phenyl)-pyrazine-2-carbonitrile

prepared by reaction with commercially available 3,4-dimethyl-phenyl-boronic acid LC-MS: t_{R}=1.05 min; [M+H+MeCN]⁺=251.26

### 3-(3-Methoxy-phenyl)-pyrazine-2-carbonitrile

prepared by reaction with commercially available 3-methoxy-phenyl-boronic acid LC-MS: t_{R}=0.85 min; [M+H]⁺=212.82

### A.6.2 Synthesis of pyrazine-2-carboxylic acid derivatives

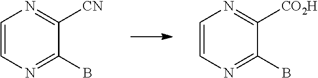

A mixture of the respective pyrazine-2-carbonitrile derivative (26 mmol), aq. 4N NaOH (190 mL) in MeOH (110 mL) was stirred at reflux for 12 h. After cooling to rt, the reaction mixture was concentrated in vacuo, the aq. residue was acidified with conc. HCl until pH 2. The resulting precipitate was filtered off and dried to yield the desired pyrazine-2-carboxylic acid derivative which was used for the next step without further purification

### 3-m-Tolyl-pyrazine-2-carboxylic acid

prepared by reaction with commercially available 3-m-tolyl-boronic acid LC-MS: t_{R}=0.28 min; [M−H]⁺=213.21

### 3-(3,4-Dimethyl-phenyl)-pyrazine-2-carboxylic acid

prepared by reaction with commercially available 3,4-dimethyl-phenyl-boronic acid LC-MS: t_{R}=0.50 min; [M−H]⁺=227.18

### 3-(3-Methoxy-phenyl)-pyrazine-2-carboxylic acid

prepared by reaction with commercially available 3-methoxy-phenyl-boronic acid LC-MS: t_{R}=0.71 min; [M+H]⁺=231.42

### A.7 Synthesis of (2-aminomethyl-thiazolidin-3-yl)aryl-methanone derivatives

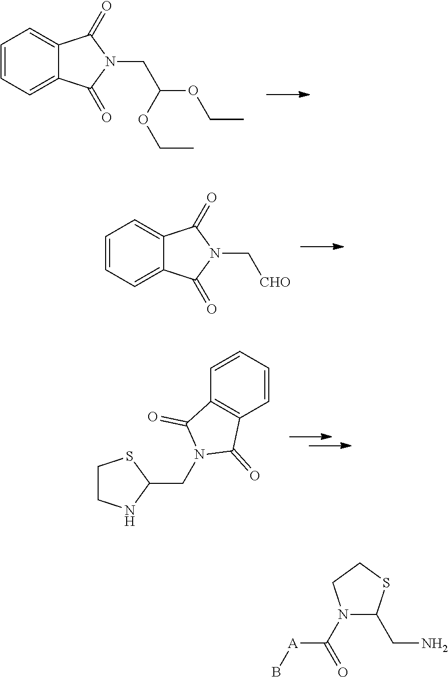

### A.7.1 Synthesis of (1,3-dioxo-1,3-dihydro-isoindol-2-yl)-acetaldehyde

To a solution of phthalimideacetaldehyde diethylacetal (10 g) in dry THF (57 mL), was added aq 6N HCl (207 mL) and the mixture was stirred at rt for 20 h. The reaction mixture was concentrated in vacuo, treated carefully with sat. NaHCO₃ solution and extracted with DCM. The combined organic extracts were dried (MgSO₄), filtered and concentrated in vacuo to yield the title compound as a white solid (4.53 g, 56%).

¹H-NMR (CDCl₃): δ=4.55 (s, 2H); 7.75 (dd, 2H); 7.91 (dd, 2H); 9.6 (s, 1H).

### A.7.2 Synthesis of 2-thiazolidin-2-ylmethyl-isoindole-1,3-dione

To a solution of (1,3-dioxo-1,3-dihydro-isoindol-2-yl)-acetaldehyde (4.53 g) in EtOH (18 mL) was added a solution of 2-aminoethanthiol hydrochloride (2.9 g) in water (3.7 mL) followed by addition in one portion of potassium acetate (2.5 g). The reaction mixture was stirred at rt for 2 h and poured into a sat. NaHCO₃ solution, the resulting precipitate was collected by filtration and washed with water and EtOH to yield the title compound as a white solid (4.92 g, 82%).

LC-MS: t_{R}=0.57 min; [M+H]⁺=248.95

### A.7.3 Synthesis of (2-aminomethyl-thiazolidin-3-yl)(hetero)aryl-methanone derivatives (general procedure)

a) To a mixture of an appropriate acid B-A-COOH (Intermediate according to A.1 to A.6) (1.7 mmol). DIPEA (8.5 mmol, 5 eq) in dry DMF (5.4 mL), was added TBTU (1.7 mmol). The mixture was stirred at rt for 15 min., then was added a solution of 2-thiazolidin-2-ylmethyl-isoindole-1,3-dione (1.7 mmol) in dry DMF (5. 4 mL), the stirring at rt was continued for 16 h. The reaction mixture was poured into water, diluted with EtOAc. The organic phase was washed with sat. NaHCO₃ solution, water, brine, dried (MgSO₄), filtered and concentrated in vacuo to yield the desired 2-(3-[(hetero)aryl-carbonyl]-thiazolidin-2-ylmethyl)-isoindole-1,3-dione derivative as a solid which was used for the next step without further purification

b) A mixture of 2-(3-[(hetero)aryl-carbonyl]-thiazolidin-2-ylmethyl)-isoindole-1,3-dione (1 mmol), hydrazine monohydrate (32 mmol) in EtOH (67 mL) was stirred at reflux for 1 h. After cooling to rt, the resulting suspension was filtered and the filtrate was concentrated in vacuo to give the desired (2-aminomethyl-thiazolidin-3-yl)(hetero)aryl-methanone derivative as a white solid which was used for the next step without further purification.

The following intermediates were synthesized according to general procedure A. 7.3 from the respective carboxylic acid B-A-COOH (Intermediate according to A.1 to A.6):

### 1) (2-Aminomethyl-thiazolidin-3-yl)-(2-methyl-5-m-tolyl-thiazol-4-yl)-methanone

LC-MS: t_{R}=0.76 min; [M+H]⁺=333.94.

### 2) (2-Aminomethyl-thiazolidin-3-yl)-[5-(4-fluoro-phenyl)-2-methyl-thiazol-4-yl]-methanone

LC-MS: t_{R}=0.71 min; [M+H]⁺=337.94.

### 3) (2-Aminomethyl-thiazolidin-3-yl)-[4-(3-chloro-phenyl)-2-methyl-thiazol-5-yl]-methanone

LC-MS: t_{R}=0.70 min; [M+H]⁺=353.91.

### 4) (2-Aminomethyl-thiazolidin-3-yl)-[4-(3-methoxy-phenyl)-2-methyl-thiazol-5-yl]-methanone

LC-MS: t_{R}=0.66 min; [M+H]⁺=349.96.

### 5) (2-Aminomethyl-thiazolidin-3-yl)-[4-(4-fluoro-phenyl)-2-methyl-thiazol-5-yl]-methanone

LC-MS: t_{R}=0.66 min; [M+H]⁺=337.87.

### 6) (2-Aminomethyl-thiazolidin-3-yl)-(2-methyl-4-p-tolyl-thiazol-5-yl)-methanone

LC-MS: t_{R}=0.66 min; [M+H]⁺=333.95.

### 7) (2-Aminomethyl-thiazolidin-3-yl)-[2-methyl-4-(3-trifluoromethyl-phenyl)-thiazol-5-yl]-methanone

LC-MS: t_{R}=0.73 min; [M+H]⁺=387.93.

### 8) (2-Aminomethyl-thiazolidin-3-yl)-(2-methyl-5-phenyl-thiazol-4-yl)-methanone

LC-MS: t_{R}=0.71 min; [M+H]⁺=319.93.

### 9) (2-Aminomethyl-thiazolidin-3-yl)-[5-(3-methoxy-phenyl)-2-methyl-thiazol-4-yl]-methanone

LC-MS: t_{R}=0.71 min; [M+H]⁺=349.95.

### 10) (2-Aminomethyl-thiazolidin-3-yl)-[5-(3-chloro-phenyl)-2-methyl-thiazol-4-yl]-methanone

LC-MS: t_{R}=0.74 min; [M+H]⁺=353.90

### 11) (2-Aminomethyl-thiazolidin-3-yl)-[2-methyl-5-(4-trifluoromethyl-phenyl)-thiazol-4-yl]-methanone

LC-MS: t_{R}=0.77 min; [M+H]⁺=387.93.

### 12) (2-Aminomethyl-thiazolidin-3-yl)-[5-(3-fluoro-4-methyl-phenyl)-thiazol-4-yl]-methanone

LC-MS: t_{R}=0.78 min; [M+H]⁺=352.06.

### 13) (2-Aminomethyl-thiazolidin-3-yl)-[5-(2,3-difluoro-phenyl)-thiazol-4-yl]-methanone

LC-MS: t_{R}=0.83 min; [M+H]⁺=356.01.

### 14) (2-Aminomethyl-thiazolidin-3-yl)-[5-(2,3-difluoro-4-methyl-phenyl)-thiazol-4-yl]-methanone

LC-MS: t_{R}=0.79 min; [M+H]⁺=370.02.

### 15) (2-Aminomethyl-thiazolidin-3-yl)-[5-(3-fluoro-phenyl)-thiazol-4-yl]-methanone

LC-MS: t_{R}=0.69 min; [M+H]⁺=324.08.

### 16) (2-Aminomethyl-thiazolidin-3-yl)-[2-cyclopropyl-5-(3-fluoro-phenyl)-thiazol-4-yl]-methanone

LC-MS: t_{R}=0.76 min; [M+H]⁺=333.94.

### 17) (2-Aminomethyl-thiazolidin-3-yl)-(2-dimethylamino-5-m-tolyl-thiazol-4-yl)-methanone

LC-MS: t_{R}=0.81 min; [M+H]⁺=362.94.

### 18) (2-Aminomethyl-thiazolidin-3-yl)-[2-dimethylamino-5-(3-methoxy-phenyl)-thiazol-4-yl]-methanone

LC-MS: t_{R}=0.75 min; [M+H]⁺=378.96.

### 19) (2-Aminomethyl-thiazolidin-3-yl)-[2-dimethylamino-5-(3-fluoro-phenyl)-thiazol-4-yl]-methanone

LC-MS: t_{R}=0.76 min; [M+H]⁺=366.94.

### 20) (2-Aminomethyl-thiazolidin-3-yl)-[2-dimethylamino-5-(4-fluoro-phenyl)-thiazol-4-yl]-methanone

LC-MS: t_{R}=0.75 min; [M+H]⁺=366.0.

### 21) (2-Aminomethyl-thiazolidin-3-yl)-[2-dimethylamino-5-(3,4-dimethyl-phenyl)-thiazol-4-yl]-methanone

LC-MS: t_{R}=0.80 min; [M+H]⁺=376.98.

### 22) (2-Aminomethyl-thiazolidin-3-yl)-(2-methyl-5-m-tolyl-oxazol-4-yl)-methanone

LC-MS: t_{R}=0.72 min; [M+H]⁺=317.94.

### 23) (2-Aminomethyl-thiazolidin-3-yl)-[2-methyl-5-(3-trifluoromethoxy-phenyl)-oxazol-4-yl]-methanone

LC-MS: t_{R}=0.81 min; [M+H]⁺=387.95.

### 24) (2-Aminomethyl-thiazolidin-3-yl)-(2-methyl-5-phenyl-oxazol-4-yl)-methanone

LC-MS: t_{R}=0.69 min; [M+H]⁺=303.95.

### 25) (2-Aminomethyl-thiazolidin-3-yl)-[5-(3-trifluoro-phenyl)-2-methyl-oxazol-4-yl]-methanone

LC-MS: t_{R}=0.71 min; [M+H]⁺=321.89.

### 26) (2-Aminomethyl-thiazolidin-3-yl)-[5-(3-chloro-phenyl)-2-methyl-oxazol-4-yl]-methanone

LC-MS: t_{R}=0.74 min; [M+H]⁺=337.89.

### 27) (2-Aminomethyl-thiazolidin-3-yl)-[5-(3-methoxy-phenyl)-2-methyl-oxazol-4-yl]-methanone

LC-MS: t_{R}=0.71 min; [M+H]⁺=349.95.

### 28) (2-Aminomethyl-thiazolidin-3-yl)-(2-m-tolyl-thiophen-3-yl)-methanone

LC-MS: t_{R}=0.80 min; [M+H]⁺=319.10.

### 29) (2-Aminomethyl-thiazolidin-3-yl)-(3′,4′-dimethyl-biphenyl-2-yl)-methanone

LC-MS: t_{R}=0.78 min; [M+H]⁺=326.99.

### 30) (2-Aminomethyl-thiazolidin-3-yl)-(3-m-tolyl-pyrazine-2-yl)-methanone

LC-MS: t_{R}=0.65 min; [M+H]⁺=314.95.

### 31) (2-Aminomethyl-thiazolidin-3-yl)-(3-(3,4-dimethyl-phenyl)-pyrazine-2-yl)-methanone

LC-MS: t_{R}=0.96 min; [M+H]⁺=328.92.

### 32) (2-Aminomethyl-thiazolidin-3-yl)-(3-(3-methoxy-phenyl)-pyrazine-2-yl)-methanone

LC-MS: t_{R}=0.63 min; [M+H]⁺=330.94.

### PREPARATION OF EXAMPLES

### General Procedure

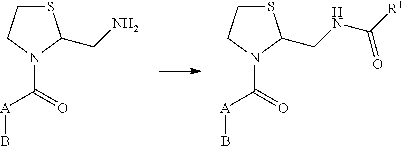

To a mixture of the respective R¹COOH derivative (0.15 mmol), DIPEA (0.75 mmol, 5 eq), in dry DMF (0.55 mL) was added TBTU (0.15 mmol). The reaction mixture was stirred at rt for 15 min. then was added a solution of the respective (2-aminomethyl-thiazolidin-3-yl)(hetero)aryl-methanone derivative (Intermediate according to A.7, or prepared in analogy to these methods) (0.15 mmol), the stirring at rt was continued for 16 h. The products were directly purified by prep. HPLC to provide the final compounds.

The following Example compounds were synthesized according to the general procedure given above:

| Example | Name | [M + H]⁺ | t_{R} |
|---|---|---|---|
| 1 | rac-2-Methyl-benzofuran-4-carboxylic acid [3-(2-methyl- | 492.10 | 0.94 |
| | 5-m-tolyl-thiazole-4-carbonyl)-thiazolidin-2-ylmethyl]- | | |
| | amide | | |
| 2 | rac-5-Chloro-2-methyl-benzofuran-4-carboxylic | 526.03 | 0.95 |
| | acid [3-(2-methyl-5-m-tolyl-thiazole-4-carbonyl)- | | |
| | thiazolidin-2-ylmethyl]-amide | | |
| 3 | rac-2-Methyl-benzooxazole-7-carboxylic acid [3-(2- | 492.98 | 0.94 |
| | methyl-5-m-tolyl-thiazole-4-carbonyl)-thiazolidin-2- | | |
| | ylmethyl]-amide | | |
| 4 | rac-Pyrrolo[2,1-b]thiazole-7-carboxylic acid [3-(2- | 483.05 | 0.83 |
| | methyl-5-m-tolyl-thiazole-4-carbonyl)-thiazolidin-2- | | |
| | ylmethyl]-amide | | |
| 5 | rac-Benzo[d]isoxazole-3-carboxylic acid [3-(2-methyl-5- | 479.09 | 0.91 |
| | m-tolyl-thiazole-4-carbonyl)-thiazolidin-2-ylmethyl]- | | |
| | amide | | |
| 6 | rac-7-Chloro-2-methyl-benzofuran-4-carboxylic | 526.02 | 0.99 |
| | acid [3-(2-methyl-5-m-tolyl-thiazole-4-carbonyl)- | | |
| | thiazolidin-2-ylmethyl]-amide | | |
| 7 | rac-3-Methyl-benzofuran-4-carboxylic acid [3-(2- | 492.10 | 0.92 |
| | methyl-5-m-tolyl-thiazole-4-carbonyl)-thiazolidin-2- | | |
| | ylmethyl]-amide | | |
| 8 | rac-2-Methyl-benzooxazole-4-carboxylic acid [3-(2- | 493.10 | 1.00 |
| | methyl-5-m-tolyl-thiazole-4-carbonyl)-thiazolidin-2- | | |
| | ylmethyl]-amide | | |
| 9 | rac-6-Methyl-pyrrolo[2,1-b]thiazole-7-carboxylic | 497.10 | 0.85 |
| | acid [3-(2-methyl-5-m-tolyl-thiazole-4-carbonyl)- | | |
| | thiazolidin-2-ylmethyl]-amide | | |
| 10 | rac-6-Methyl-imidazo[2,1-b]thiazole-5-carboxylic | 497.96 | 0.84 |
| | acid [3-(2-methyl-5-m-tolyl-thiazole-4-carbonyl)- | | |
| | thiazolidin-2-ylmethyl]-amide | | |
| 11 | rac-2-Methyl-7-trifluoromethyl-benzofuran-4- | 560.02 | 1.00 |
| | carboxylic acid [3-(2-methyl-5-m-tolyl-thiazole-4- | | |
| | carbonyl)-thiazolidin-2-ylmethyl]-amide | | |
| 12 | rac-2,3-Dimethyl-benzofuran-4-carboxylic acid [3- | 506.10 | 0.95 |
| | (2-methyl-5-m-tolyl-thiazole-4-carbonyl)-thiazolidin- | | |
| | 2-ylmethyl]-amide | | |
| 13 | rac-Imidazo[1,2-a]pyridine-3-carboxylic acid [3-(2- | 478.09 | 0.81 |
| | methyl-5-m-tolyl-thiazole-4-carbonyl)-thiazolidin-2- | | |
| | ylmethyl]-amide | | |
| 14 | rac-Imidazo[2,1-b]thiazole-5-carboxylic acid [3-(2-methyl- | 483.96 | 0.86 |
| | 5-m-tolyl-thiazole-4-carbonyl)-thiazolidin-2-ylmethyl]- | | |
| | amide | | |
| 15 | rac-7-Fluoro-2-methyl-benzofuran-4-carboxylic acid | 510.07 | 0.95 |
| | [3-(2-methyl-5-m-tolyl-thiazole-4-carbonyl)- | | |
| | thiazolidin-2-ylmethyl]-amide | | |
| 16 | rac-2,3-Dihydro-benzofuran-4-carboxylic acid [3-(2- | 480.13 | 0.89 |
| | methyl-5-m-tolyl-thiazole-4-carbonyl)-thiazolidin-2- | | |
| | ylmethyl]-amide | | |
| 17 | rac-Benzooxazole-4-carboxylic acid [3-(2-methyl-5- | 479.10 | 0.87 |
| | m-tolyl-thiazole-4-carbonyl)thiazolidin-2-ylmethyl]- | | |
| | amide | | |
| 18 | rac-2-Methyl-imidazo[1,2-a]pyridine-3-carboxylic | 492.08 | 0.81 |
| | acid [3-(2-methyl-5-m-tolyl-thiazole-4-carbonyl)- | | |
| | thiazolidin-2-ylmethyl]-amide | | |
| 19 | rac-2-Methyl-6-trifluoromethyl-benzofuran-4- | 560.05 | 1.10 |
| | carboxylic acid [3-(2-methyl-5-m-tolyl-thiazole-4- | | |
| | carbonyl)-thiazolidin-2-ylmethyl]-amide | | |
| 20 | rac-6-Fluoro-2-methyl-benzofuran-4-carboxylic acid | 510.09 | 0.96 |
| | [3-(2-methyl-5-m-tolyl-thiazole-4-carbonyl)- | | |
| | thiazolidin-2-ylmethyl]-amide | | |
| 21 | rac-2,3-Dihydro-benzo[1,4]dioxine-5-carboxylic | 496.08 | 0.88 |
| | acid [3-(2-methyl-5-m-tolyl-thiazole-4-carbonyl)- | | |
| | thiazolidin-2-ylmethyl]-amide | | |
| 22 | rac-Benzo[1,2,5]oxadiazole-4-carboxylic acid [3-(2- | 480.06 | 0.89 |
| | methyl-5-m-tolyl-thiazole-4-carbonyl)-thiazolidin-2- | | |
| | ylmethyl]-amide | | |
| 23 | rac-1-Methyl-1H-indazole-3-carboxylic acid [3-(2- | 492.10 | 0.91 |
| | methyl-5-m-tolyl-thiazole-4-carbonyl)-thiazolidin-2- | | |
| | ylmethyl]-amide | | |
| 24 | rac-6-Chloro-2-methyl-benzofuran-4-carboxylic | 526.04 | 0.91 |
| | acid [3-(2-methyl-5-m-tolyl-thiazole-4-carbonyl)- | | |
| | thiazolidin-2-ylmethyl]-amide | | |
| 25 | rac-Benzooxazole-7-carboxylic acid [3-(2-methyl-5- | 479.07 | 1.00 |
| | m-tolyl-thiazole-4-carbonyl)thiazolidin-2-ylmethyl]- | | |
| | amide | | |
| 26 | rac-Benzo[1,2,5]thiadiazole-4-carboxylic acid [3-(2- | 496.03 | 0.90 |
| | methyl-5-m-tolyl-thiazole-4-carbonyl)-thiazolidin-2- | | |
| | ylmethyl]-amide | | |
| 27 | rac-Benzo[d]isothiazole-3-carboxylic acid [3-(2- | 495.08 | 0.98 |
| | methyl-5-m-tolyl-thiazole-4-carbonyl)-thiazolidin-2- | | |
| | ylmethyl]-amide | | |
| 28 | rac-Benzothiazole-7-carboxylic acid [3-(2-methyl-5- | 495.07 | 0.84 |
| | m-tolyl-thiazole-4-carbonyl)thiazolidin-2-ylmethyl]- | | |
| | amide | | |
| 29 | rac-2,3-Dihydro-benzofuran-4-carboxylic acid {3-[4- | 499.95 | 0.98 |
| | (3-chloro-phenyl)-2-methyl-thiazole-5-carbonyl]- | | |
| | thiazolidin-2-ylmethyl}-amide | | |
| 30 | rac-1-Methyl-1H-indazole-3-carboxylic acid {3-[4- | 511.99 | 1.01 |
| | (3-chloro-phenyl)-2-methyl-thiazole-5-carbonyl]- | | |
| | thiazolidin-2-ylmethyl}-amide | | |
| 31 | rac-Benzothiazole-7-carboxylic acid {3-[4-(3- | 510.94 | 0.92 |
| | methoxy-phenyl)-2-methyl-thiazole-5-carbonyl]- | | |
| | thiazolidin-2-ylmethyl}-amide | | |
| 32 | rac-2,3-Dihydro-benzofuran-4-carboxylic acid {3-[4- | 496 | 0.93 |
| | (3-methoxy-phenyl)-2-methyl-thiazole-5-carbonyl]- | | |
| | thiazolidin-2-ylmethyl}-amide | | |
| 33 | rac-1-Methyl-1H-indazole-3-carboxylic acid {3-[4- | 508.02 | 0.96 |
| | (3-methoxy-phenyl)-2-methyl-thiazole-5-carbonyl]- | | |
| | thiazolidin-2-ylmethyl}-amide | | |
| 34 | rac-Benzothiazole-7-carboxylic acid {3-[4-(3-chloro- | 515 | 0.97 |
| | phenyl)-2-methyl-thiazole-5-carbonyl]-thiazolidin-2- | | |
| | ylmethyl}-amide | | |
| 35 | rac-Imidazo[1,2-a]pyridine-3-carboxylic acid {3-[4- | 497.98 | 0.79 |
| | (3-chloro-phenyl)-2-methyl-thiazole-5-carbonyl]- | | |
| | thiazolidin-2-ylmethyl}-amide | | |
| 36 | rac-2,3-Dihydro-benzofuran-4-carboxylic acid {3-[4- | 483.96 | 0.95 |
| | (4-fluoro-phenyl)-2-methyl-thiazole-5-carbonyl]- | | |
| | thiazolidin-2-ylmethyl}-amide | | |
| 37 | rac-2,3-Dihydro-benzofuran-4-carboxylic acid [3-(2- | 479.97 | 0.96 |
| | methyl-4-p-tolyl-thiazole-5-carbonyl)-thiazolidin-2- | | |
| | ylmethyl]-amide | | |
| 38 | rac-2,3-Dihydro-benzo[1,4]dioxine-5-carboxylic | 515.96 | 0.98 |
| | acid {3-[4-(3-chloro-phenyl)-2-methyl-thiazole-5- | | |
| | carbonyl]-thiazolidin-2-ylmethyl}-amide | | |
| 39 | rac-1-Methyl-1H-indazole-3-carboxylic acid {3-[4- | 496 | 0.97 |
| | (4-fluoro-phenyl)-2-methyl-thiazole-5-carbonyl]- | | |
| | thiazolidin-2-ylmethyl}-amide | | |
| 40 | rac-Benzothiazole-7-carboxylic acid {3-[4-(4-fluoro- | 498.98 | 0.93 |
| | phenyl)-2-methyl-thiazole-5-carbonyl]-thiazolidin-2- | | |
| | ylmethyl}-amide | | |
| 41 | rac-1-Methyl-1H-indazole-3-carboxylic acid [3-(2-methyl- | 492.02 | 0.99 |
| | 4-p-tolyl-thiazole-5-carbonyl)-thiazolidin-2-ylmethyl]- | | |
| | amide | | |
| 42 | rac-2,3-Dihydro-benzo[1,4]dioxine-5-carboxylic | 495.99 | 0.96 |
| | acid [3-(2-methyl-4-p-tolyl-thiazole-5-carbonyl)- | | |
| | thiazolidin-2-ylmethyl]-amide | | |
| 43 | rac-2,3-Dihydro-benzo[1,4]dioxine-5-carboxylic | 512.05 | 0.93 |
| | acid {3-[4-(3-methoxy-phenyl)-2-methyl-thiazole-5- | | |
| | carbonyl]-thiazolidin-2-ylmethyl}-amide | | |
| 44 | rac-2,3-Dihydro-benzo[1,4]dioxine-5-carboxylic | 499.97 | 0.95 |
| | acid {3-[4-(4-fluoro-phenyl)-2-methyl-thiazole-5- | | |
| | carbonyl]-thiazolidin-2-ylmethyl}-amide | | |
| 45 | rac-Imidazo[1,2-a]pyridine-3-carboxylic acid {3-[4- | 493.98 | 0.75 |
| | (3-methoxy-phenyl)-2-methyl-thiazole-5-carbonyl]- | | |
| | thiazolidin-2-ylmethyl}-amide | | |
| 46 | rac-Imidazo[1,2-a]pyridine-3-carboxylic acid {3-[4- | 481.96 | 0.75 |
| | (4-fluoro-phenyl)-2-methyl-thiazole-5-carbonyl]- | | |
| | thiazolidin-2-ylmethyl}-amide | | |
| 47 | rac-Imidazo[1,2-a]pyridine-3-carboxylic acid [3-(2- | 477.99 | 0.77 |
| | methyl-4-p-tolyl-thiazole-5-carbonyl)-thiazolidin-2- | | |
| | ylmethyl]-amide | | |
| 48 | rac-Benzothiazole-7-carboxylic acid {3-[2-methyl-4- | 549 | 1.01 |
| | (3-trifluoromethyl-phenyl)-thiazole-5-carbonyl]- | | |
| | thiazolidin-2-ylmethyl}-amide | | |
| 49 | rac-2,3-Dihydro-benzo[1,4]dioxine-5-carboxylic | 550 | 1.01 |
| | acid {3-[2-methyl-4-(3-trifluoromethyl-phenyl)- | | |
| | thiazole-5-carbonyl]-thiazolidin-2-ylmethyl}-amide | | |
| 50 | rac-1-Methyl-1H-indazole-3-carboxylic acid {3-[2- | 546.02 | 1.03 |
| | methyl-4-(3-trifluoromethyl-phenyl)-thiazole-5- | | |
| | carbonyl]-thiazolidin-2-ylmethyl}-amide | | |
| 51 | rac-Imidazo[1,2-a]pyridine-3-carboxylic acid {3-[2- | 532.01 | 0.82 |
| | methyl-4-(3-trifluoromethyl-phenyl)-thiazole-5- | | |
| | carbonyl]-thiazolidin-2-ylmethyl}-amide | | |
| 52 | rac-2,3-Dihydro-benzofuran-4-carboxylic acid [3-(2- | 465.98 | 0.95 |
| | methyl-5-phenyl-thiazole-4-carbonyl)-thiazolidin-2- | | |
| | ylmethyl]-amide | | |
| 53 | rac-2,3-Dihydro-benzo[1,4]dioxine-5-carboxylic | 512.05 | 0.95 |
| | acid {3-[5-(3-methoxy-phenyl)-2-methyl-thiazole-4- | | |
| | carbonyl]-thiazolidin-2-ylmethyl}-amide | | |
| 54 | rac-Benzothiazole-7-carboxylic acid {3-[5-(3- | 510.95 | 0.95 |
| | methoxy-phenyl)-2-methyl-thiazole-4-carbonyl]- | | |
| | thiazolidin-2-ylmethyl}-amide | | |
| 55 | rac-2,3-Dihydro-benzofuran-4-carboxylic acid {3-[5- | 496.01 | 0.95 |
| | (3-methoxy-phenyl)-2-methyl-thiazole-4-carbonyl]- | | |
| | thiazolidin-2-ylmethyl}-amide | | |
| 56 | rac-Imidazo[1,2-a]pyridine-3-carboxylic acid {3-[5- | 493.99 | 0.77 |
| | (3-methoxy-phenyl)-2-methyl-thiazole-4-carbonyl]- | | |
| | thiazolidin-2-ylmethyl}-amide | | |
| 57 | rac-2,3-Dihydro-benzo[1,4]dioxine-5-carboxylic | 481.98 | 0.94 |
| | acid [3-(2-methyl-5-phenyl-thiazole-4-carbonyl)- | | |
| | thiazolidin-2-ylmethyl]-amide | | |
| 58 | rac-2,3-Dihydro-benzofuran-4-carboxylic acid {3-[5- | 499.94 | 0.99 |
| | (3-chloro-phenyl)-2-methyl-thiazole-4-carbonyl]- | | |
| | thiazolidin-2-ylmethyl}-amide | | |
| 59 | rac-2,3-Dihydro-benzofuran-4-carboxylic acid {3-[2- | 533.99 | 1.01 |
| | methyl-5-(4-trifluoromethyl-phenyl)-thiazole-4- | | |
| | carbonyl]-thiazolidin-2-ylmethyl}-amide | | |
| 60 | rac-2,3-Dihydro-benzo[1,4]dioxine-5-carboxylic | 550 | 1.01 |
| | acid {3-[2-methyl-5-(4-trifluoromethyl-phenyl)- | | |
| | thiazole-4-carbonyl]-thiazolidin-2-ylmethyl}-amide | | |
| 61 | rac-1-Methyl-1H-indazole-3-carboxylic acid {3-[2- | 546.04 | 1.03 |
| | methyl-5-(4-trifluoromethyl-phenyl)-thiazole-4- | | |
| | carbonyl]-thiazolidin-2-ylmethyl}-amide | | |
| 62 | rac-Imidazo[1,2-a]pyridine-3-carboxylic acid {3-[2- | 531.99 | 0.83 |
| | methyl-5-(4-trifluoromethyl-phenyl)-thiazole-4- | | |
| | carbonyl]-thiazolidin-2-ylmethyl}-amide | | |
| 63 | rac-Chroman-8-carboxylic acid [3-(2-methyl-5-m- | 494.02 | 1.01 |
| | tolyl-thiazole-4-carbonyl)-thiazolidin-2-ylmethyl]- | | |
| | amide | | |
| 64 | rac-Chroman-5-carboxylic acid [3-(2-methyl-5-m- | 494.01 | 0.99 |
| | tolyl-thiazole-4-carbonyl)-thiazolidin-2-ylmethyl]- | | |
| | amide | | |
| 65 | rac-3,4-Dihydro-2H-benzo[1,4]oxazine-5-carboxylic | 495.04 | 1.00 |
| | acid [3-(2-methyl-5-m-tolyl-thiazole-4-carbonyl)- | | |
| | thiazolidin-2-ylmethyl]-amide | | |
| 66 | rac-3,4-Dihydro-2H-benzo[1,4]oxazine-8-carboxylic | 495.02 | 0.92 |
| | acid [3-(2-methyl-5-m-tolyl-thiazole-4-carbonyl)- | | |
| | thiazolidin-2-ylmethyl]-amide | | |
| 67 | rac-4-Methyl-3,4-dihydro-2H-benzo[1,4]oxazine-8- | 509.06 | 0.99 |
| | carboxylic acid [3-(2-methyl-5-m-tolyl-thiazole-4- | | |
| | carbonyl)-thiazolidin-2-ylmethyl]-amide | | |
| 68 | rac-4-Methyl-3,4-dihydro-2H-benzo[1,4]oxazine-5- | 509.05 | 0.96 |
| | carboxylic acid [3-(2-methyl-5-m-tolyl-thiazole-4- | | |
| | carbonyl)-thiazolidin-2-ylmethyl]-amide | | |
| 69 | rac-3-Oxo-3,4-dihydro-2H-benzo[1,4]oxazine-8- | 509.06 | 0.87 |
| | carboxylic acid [3-(2-methyl-5-m-tolyl-thiazole-4- | | |
| | carbonyl)-thiazolidin-2-ylmethyl]-amide | | |
| 70 | rac-4-Methyl-3-oxo-3,4-dihydro-2H- | 523.05 | 0.93 |
| | benzo[1,4]oxazine-8-carboxylic acid [3-(2-methyl-5- | | |
| | m-tolyl-thiazole-4-carbonyl)-thiazolidin-2-ylmethyl]- | | |
| | amide | | |
| 71 | rac-2,3-Dihydro-benzofuran-4-carboxylic acid [3- | 473 | 1.04 |
| | (3′,4′-dimethyl-biphenyl-2-carbonyl)-thiazolidin-2- | | |
| | ylmethyl]-amide | | |
| 72 | rac-Benzothiazole-7-carboxylic acid [3-(3′,4′- | 488.01 | 1.06 |
| | dimethyl-biphenyl-2-carbonyl)-thiazolidin-2- | | |
| | ylmethyl]-amide | | |
| 73 | rac-2,3-Dihydro-benzo[1,4]dioxine-5-carboxylic | 489.05 | 1.04 |
| | acid [3-(3′,4′-dimethyl-biphenyl-2-carbonyl)- | | |
| | thiazolidin-2-ylmethyl]-amide | | |
| 74 | rac-2,3-Dihydro-benzofuran-4-carboxylic acid {3-[2- | 534 | 1.01 |
| | methyl-4-(3-trifluoromethyl-phenyl)-thiazole-5- | | |
| | carbonyl]-thiazolidin-2-ylmethyl}-amide | | |
| 75 | rac-2,3-Dihydro-benzo[1,4]dioxine-5-carboxylic | 497.97 | 0.95 |
| | acid {3-[5-(4-fluoro-phenyl)-2-methyl-thiazole-4- | | |
| | carbonyl]-thiazolidin-2-ylmethyl}-amide | | |
| 76 | rac-Imidazo[1,2-a]pyridine-3-carboxylic acid {3-[5- | 481.95 | 0.77 |
| | (4-fluoro-phenyl)-2-methyl-thiazole-4-carbonyl]- | | |
| | thiazolidin-2-ylmethyl}-amide | | |
| 77 | rac-2,3-Dihydro-benzofuran-4-carboxylic acid {3-[5- | 483.93 | 0.95 |
| | (4-fluoro-phenyl)-2-methyl-thiazole-4-carbonyl]- | | |
| | thiazolidin-2-ylmethyl}-amide | | |
| 78 | rac-Benzothiazole-7-carboxylic acid {3-[5-(4-fluoro- | 498.95 | 0.95 |
| | phenyl)-2-methyl-thiazole-4-carbonyl]-thiazolidin-2- | | |
| | ylmethyl}-amide | | |
| 79 | rac-1-Methyl-1H-indazole-3-carboxylic acid [3-(2- | 477.95 | 0.96 |
| | methyl-5-phenyl-thiazole-4-carbonyl)-thiazolidin-2- | | |
| | ylmethyl]-amide | | |
| 80 | rac-2,3-Dihydro-benzofuran-4-carboxylic acid [3-(2- | 463.98 | 0.96 |
| | methyl-5-m-tolyl-oxazole-4-carbonyl)-thiazolidin-2- | | |
| | ylmethyl]-amide | | |
| 81 | rac-2,3-Dihydro-benzo[1,4]dioxine-5-carboxylic | 480.03 | 0.96 |
| | acid [3-(2-methyl-5-m-tolyl-oxazole-4-carbonyl)- | | |
| | thiazolidin-2-ylmethyl]-amide | | |
| 82 | rac-1-Methyl-1H-indazole-3-carboxylic acid [3- | 485.04 | 1.06 |
| | (3′,4′-dimethyl-biphenyl-2-carbonyl)-thiazolidin-2- | | |
| | ylmethyl]-amide | | |
| 83 | rac-Benzothiazole-7-carboxylic acid [3-(2-methyl-5- | 478.93 | 0.95 |
| | m-tolyl-oxazole-4-carbonyl)-thiazolidin-2-ylmethyl]- | | |
| | amide | | |
| 84 | rac-Imidazo[1,2-a]pyridine-3-carboxylic acid [3-(2- | 461.98 | 0.74 |
| | methyl-5-m-tolyl-oxazole-4-carbonyl)-thiazolidin-2- | | |
| | ylmethyl]-amide | | |
| 85 | rac-2,3-Dihydro-benzofuran-4-carboxylic acid {3-[2- | 534.04 | 1.02 |
| | methyl-5-(3-trifluoromethoxy-phenyl)-oxazole-4- | | |
| | carbonyl]-thiazolidin-2-ylmethyl}-amide | | |
| 86 | rac-2,3-Dihydro-benzo[1,4]dioxine-5-carboxylic | 549.02 | 0.91 |
| | acid {3-[2-methyl-5-(3-trifluoromethoxy-phenyl)- | | |
| | oxazole-4-carbonyl]-thiazolidin-2-ylmethyl}-amide | | |
| 87 | rac-1-Methyl-1H-indazole-3-carboxylic acid {3-[2- | 545.9 | 0.93 |
| | methyl-5-(3-trifluoromethoxy-phenyl)-oxazole-4- | | |
| | carbonyl]-thiazolidin-2-ylmethyl}-amide | | |
| 88 | rac-Benzothiazole-7-carboxylic acid [3-(2-methyl-5- | 465.04 | 0.79 |
| | phenyl-oxazole-4-carbonyl)-thiazolidin-2-ylmethyl]- | | |
| | amide | | |
| 89 | rac-1-Methyl-1H-indazole-3-carboxylic acid [3-(2- | 462.05 | 0.85 |
| | methyl-5-phenyl-oxazole-4-carbonyl)-thiazolidin-2- | | |
| | ylmethyl]-amide | | |
| 90 | rac-2,3-Dihydro-benzo[1,4]dioxine-5-carboxylic | 466.04 | 0.83 |
| | acid [3-(2-methyl-5-phenyl-oxazole-4-carbonyl)- | | |
| | thiazolidin-2-ylmethyl]-amide | | |
| 91 | rac-2,3-Dihydro-benzofuran-4-carboxylic acid [3-(2- | 450.01 | 0.84 |
| | methyl-5-phenyl-oxazole-4-carbonyl)-thiazolidin-2- | | |
| | ylmethyl]-amide | | |
| 92 | rac-Imidazo[1,2-a]pyridine-3-carboxylic acid [3-(2- | 448.01 | 0.74 |
| | methyl-5-phenyl-oxazole-4-carbonyl)-thiazolidin-2- | | |
| | ylmethyl]-amide | | |
| 93 | rac-Benzothiazole-7-carboxylic acid {3-[5-(3-fluoro- | 482.90 | 0.80 |
| | phenyl)-2-methyl-oxazole-4-carbonyl]-thiazolidin-2- | | |
| | ylmethyl}-amide | | |
| 94 | rac-2,3-Dihydro-benzofuran-4-carboxylic acid {3-[5- | 468.04 | 0.86 |
| | (3-fluoro-phenyl)-2-methyl-oxazole-4-carbonyl]- | | |
| | thiazolidin-2-ylmethyl}-amide | | |
| 95 | rac-2,3-Dihydro-benzo[1,4]dioxine-5-carboxylic | 484.04 | 0.84 |
| | acid {3-[5-(3-fluoro-phenyl)-2-methyl-oxazole-4- | | |
| | carbonyl]-thiazolidin-2-ylmethyl}-amide | | |
| 96 | rac-1-Methyl-1H-indazole-3-carboxylic acid {3-[5- | 480.02 | 0.86 |
| | (3-fluoro-phenyl)-2-methyl-oxazole-4-carbonyl]- | | |
| | thiazolidin-2-ylmethyl}-amide | | |
| 97 | rac-Imidazo[1,2-a]pyridine-3-carboxylic acid {3-[5- | 466.03 | 0.75 |
| | (3-fluoro-phenyl)-2-methyl-oxazole-4-carbonyl]- | | |
| | thiazolidin-2-ylmethyl}-amide | | |
| 98 | rac-Benzothiazole-7-carboxylic acid {3-[5-(3-chloro- | 498.90 | 0.83 |
| | phenyl)-2-methyl-oxazole-4-carbonyl]-thiazolidin-2- | | |
| | ylmethyl}-amide | | |
| 99 | rac-1-Methyl-1H-indazole-3-carboxylic acid {3-[5- | 496.03 | 0.90 |
| | (3-chloro-phenyl)-2-methyl-oxazole-4-carbonyl]- | | |
| | thiazolidin-2-ylmethyl}-amide | | |
| 100 | rac-2,3-Dihydro-benzofuran-4-carboxylic acid {3-[5- | 484.01 | 0.89 |
| | (3-chloro-phenyl)-2-methyl-oxazole-4-carbonyl]- | | |
| | thiazolidin-2-ylmethyl}-amide | | |
| 101 | rac-2,3-Dihydro-benzo[1,4]dioxine-5-carboxylic | 499.98 | 0.88 |
| | acid {3-[5-(3-chloro-phenyl)-2-methyl-oxazole-4- | | |
| | carbonyl]-thiazolidin-2-ylmethyl}-amide | | |
| 102 | rac-Imidazo[1,2-a]pyridine-3-carboxylic acid {3-[5- | 481.97 | 0.79 |
| | (3-chloro-phenyl)-2-methyl-oxazole-4-carbonyl]- | | |
| | thiazolidin-2-ylmethyl}-amide | | |
| 103 | rac-2,3-Dihydro-benzofuran-4-carboxylic acid {3-[5- | 480.01 | 0.84 |
| | (3-methoxy-phenyl)-2-methyl-oxazole-4-carbonyl]- | | |
| | thiazolidin-2-ylmethyl}-amide | | |
| 104 | rac-Benzothiazole-7-carboxylic acid {3-[5-(3- | 494.95 | 0.78 |
| | methoxy-phenyl)-2-methyl-oxazole-4-carbonyl]- | | |
| | thiazolidin-2-ylmethyl}-amide | | |
| 105 | rac-2,3-Dihydro-benzo[1,4]dioxine-5-carboxylic | 496.01 | 0.83 |
| | acid {3-[5-(3-methoxy-phenyl)-2-methyl-oxazole-4- | | |
| | carbonyl]-thiazolidin-2-ylmethyl}-amide | | |
| 106 | rac-1-Methyl-1H-indazole-3-carboxylic acid {3-[5- | 492.03 | 0.84 |
| | (3-methoxy-phenyl)-2-methyl-oxazole-4-carbonyl]- | | |
| | thiazolidin-2-ylmethyl}-amide | | |
| 107 | rac-Imidazo[1,2-a]pyridine-3-carboxylic acid {3-[5- | 478.03 | 0.78 |
| | (3-methoxy-phenyl)-2-methyl-oxazole-4-carbonyl]- | | |
| | thiazolidin-2-ylmethyl}-amide | | |
| 108 | rac-2,3-Dihydro-benzofuran-4-carboxylic acid [3-(2- | 465.02 | 0.92 |
| | m-tolyl-thiophene-3-carbonyl)-thiazolidin-2- | | |
| | ylmethyl]-amide | | |
| 109 | rac-Benzothiazole-7-carboxylic acid [3-(2-m-tolyl- | 479.98 | 0.89 |
| | thiophene-3-carbonyl)-thiazolidin-2-ylmethyl]-amide | | |
| 110 | rac-2,3-Dihydro-benzo[1,4]dioxine-5-carboxylic | 480.99 | 0.92 |
| | acid [3-(2-m-tolyl-thiophene-3-carbonyl)-thiazolidin- | | |
| | 2-ylmethyl]-amide | | |
| 111 | rac-Benzothiazole-7-carboxylic acid {3-[5-(3-fluoro- | 484.93 | 0.79 |
| | phenyl)-thiazole-4-carbonyl]-thiazolidin-2-ylmethyl}- | | |
| | amide | | |
| 112 | rac-1-Methyl-1H-indazole-3-carboxylic acid {3-[5- | 481.97 | 0.85 |
| | (3-fluoro-phenyl)-thiazole-4-carbonyl]-thiazolidin-2- | | |
| | ylmethyl}-amide | | |
| 113 | rac-2,3-Dihydro-benzofuran-4-carboxylic acid {3-[5- | 470.08 | 0.83 |
| | (3-fluoro-phenyl)-thiazole-4-carbonyl]-thiazolidin-2- | | |
| | ylmethyl}-amide | | |
| 114 | rac-2,3-Dihydro-benzo[1,4]dioxine-5-carboxylic | 485.98 | 0.82 |
| | acid {3-[5-(3-fluoro-phenyl)-thiazole-4-carbonyl]- | | |
| | thiazolidin-2-ylmethyl}-amide | | |
| 115 | rac-Imidazo[1,2-a]pyridine-3-carboxylic acid {3-[5- | 467.97 | 0.75 |
| | (3-fluoro-phenyl)-thiazole-4-carbonyl]-thiazolidin-2- | | |
| | ylmethyl}-amide | | |
| 116 | rac-2,3-Dihydro-benzofuran-4-carboxylic acid {3-[2- | 509.99 | 0.92 |
| | cyclopropyl-5-(3-fluoro-phenyl)-thiazole-4- | | |
| | carbonyl]-thiazolidin-2-ylmethyl}-amide | | |
| 117 | rac-2,3-Dihydro-benzo[1,4]dioxine-5-carboxylic | 525.98 | 0.92 |
| | acid {3-[2-cyclopropyl-5-(3-fluoro-phenyl)-thiazole- | | |
| | 4-carbonyl]-thiazolidin-2-ylmethyl}-amide | | |
| 118 | rac-2,3-Dihydro-benzo[1,4]dioxine-5-carboxylic | 513.99 | 0.89 |
| | acid {3-[5-(3-fluoro-4-methyl-phenyl)-2-methyl- | | |
| | thiazole-4-carbonyl]-thiazolidin-2-ylmethyl}-amide | | |
| 119 | rac-2,3-Dihydro-benzofuran-4-carboxylic acid {3-[5- | 497.99 | 0.90 |
| | (3-fluoro-4-methyl-phenyl)-2-methyl-thiazole-4- | | |
| | carbonyl]-thiazolidin-2-ylmethyl}-amide | | |
| 120 | rac-Benzothiazole-7-carboxylic acid {3-[5-(3-fluoro- | 512.98 | 0.84 |
| | 4-methyl-phenyl)-2-methyl-thiazole-4-carbonyl]- | | |
| | thiazolidin-2-ylmethyl}-amide | | |
| 121 | rac-2,3-Dihydro-benzofuran-4-carboxylic acid {3-[5-(3,4- | 501.98 | 0.87 |
| | difluoro-phenyl)-2-methyl-thiazole-4-carbonyl]-thiazolidin- | | |
| | 2-ylmethyl}-amide | | |
| 122 | rac-2,3-Dihydro-benzo[1,4]dioxine-5-carboxylic | 517.95 | 0.87 |
| | acid {3-[5-(3,4-difluoro-phenyl)-2-methyl-thiazole-4- | | |
| | carbonyl]-thiazolidin-2-ylmethyl}-amide | | |
| 123 | rac-Benzothiazole-7-carboxylic acid {3-[5-(2,3- | 530.93 | 0.87 |
| | difluoro-4-methyl-phenyl)-2-methyl-thiazole-4- | | |
| | carbonyl]-thiazolidin-2-ylmethyl}-amide | | |
| 124 | rac-Imidazo[1,2-a]pyridine-3-carboxylic acid {3-[5- | 513.96 | 0.83 |
| | (2,3-difluoro-4-methyl-phenyl)-2-methyl-thiazole-4- | | |
| | carbonyl]-thiazolidin-2-ylmethyl}-amide | | |
| 125 | rac-2,3-Dihydro-benzofuran-4-carboxylic acid {3-[5- | 516 | 0.91 |
| | (2,3-difluoro-4-methyl-phenyl)-2-methyl-thiazole-4- | | |
| | carbonyl]-thiazolidin-2-ylmethyl}-amide | | |
| 126 | rac-1-Methyl-1H-indazole-3-carboxylic acid {3-[5- | 528 | 0.93 |
| | (2,3-difluoro-4-methyl-phenyl)-2-methyl-thiazole-4- | | |
| | carbonyl]-thiazolidin-2-ylmethyl}-amide | | |
| 127 | rac-2,3-Dihydro-benzo[1,4]dioxine-5-carboxylic | 531.94 | 0.91 |
| | acid {3-[5-(2,3-difluoro-4-methyl-phenyl)-2-methyl- | | |
| | thiazole-4-carbonyl]-thiazolidin-2-ylmethyl}-amide | | |
| 128 | rac-2,3-Dihydro-benzofuran-4-carboxylic acid [3-(2- | 509.02 | 0.91 |
| | dimethylamino-5-m-tolyl-thiazole-4-carbonyl)- | | |
| | thiazolidin-2-ylmethyl]-amide | | |
| 129 | rac-Imidazo[1,2-a]pyridine-3-carboxylic acid [3- | 471.02 | 0.85 |
| | (3′,4′-dimethyl-biphenyl-2-carbonyl)-thiazolidin-2- | | |
| | ylmethyl]-amide | | |
| 130 | rac-1H-Indazole-7-carboxylic acid [3-(2-methyl-5- | 477.96 | 0.95 |
| | m-tolyl-thiazole-4-carbonyl)-thiazolidin-2-ylmethyl]- | | |
| | amide | | |
| 131 | rac-6-Fluoro-4H-benzo[1,3]dioxine-8-carboxylic | 514.01 | 0.99 |
| | acid [3-(2-methyl-5-m-tolyl-thiazole-4-carbonyl)- | | |
| | thiazolidin-2-ylmethyl]-amide | | |
| 132 | rac-1-Methyl-1H-indole-4-carboxylic acid [3-(2- | 491.01 | 0.99 |
| | methyl-5-m-tolyl-thiazole-4-carbonyl)-thiazolidin-2- | | |
| | ylmethyl]-amide | | |
| 133 | rac-1-Methyl-1H-indole-7-carboxylic acid [3-(2- | 491.03 | 1.01 |
| | methyl-5-m-tolyl-thiazole-4-carbonyl)-thiazolidin-2- | | |
| | ylmethyl]-amide | | |
| 134 | rac-2,3-Dihydro-benzofuran-7-carboxylic acid [3-(2- | 480.03 | 0.98 |
| | methyl-5-m-tolyl-thiazole-4-carbonyl)-thiazolidin-2- | | |
| | ylmethyl]-amide | | |
| 135 | rac-1-Methyl-1H-indole-3-carboxylic acid [3-(2- | 491.01 | 0.98 |
| | methyl-5-m-tolyl-thiazole-4-carbonyl)-thiazolidin-2- | | |
| | ylmethyl]-amide | | |
| 136 | rac-5-Chloro-1,3-dimethyl-1H-pyrazole-4-carboxylic | 489.92 | 0.92 |
| | acid [3-(2-methyl-5-m-tolyl-thiazole-4-carbonyl)- | | |
| | thiazolidin-2-ylmethyl]-amide | | |
| 137 | rac-3-Ethyl-5-methyl-isoxazole-4-carboxylic acid [3- | 470.93 | 0.96 |
| | (2-methyl-5-m-tolyl-thiazole-4-carbonyl)-thiazolidin- | | |
| | 2-ylmethyl]-amide | | |
| 138 | rac-1-Ethyl-3-methyl-1H-pyrazole-4-carboxylic acid | 469.84 | 0.88 |
| | [3-(2-methyl-5-m-tolyl-thiazole-4-carbonyl)- | | |
| | thiazolidin-2-ylmethyl]-amide | | |
| 139 | rac-1H-Benzoimidazole-4-carboxylic acid [3-(2- | 477.96 | 0.77 |
| | methyl-5-m-tolyl-thiazole-4-carbonyl)-thiazolidin-2- | | |
| | ylmethyl]-amide | | |
| 140 | rac-5-Ethyl-3-methyl-isoxazole-4-carboxylic acid [3- | 470.83 | 0.97 |
| | (2-methyl-5-m-tolyl-thiazole-4-carbonyl)-thiazolidin- | | |
| | 2-ylmethyl]-amide | | |
| 141 | rac-3,5-Dimethyl-isoxazole-4-carboxylic acid [3-(2- | 456.90 | 0.93 |
| | methyl-5-m-tolyl-thiazole-4-carbonyl)-thiazolidin-2- | | |
| | ylmethyl]-amide | | |
| 142 | rac-1-Methyl-5-trifluoromethyl-1H-pyrazole-4- | 509.99 | 0.95 |
| | carboxylic acid [3-(2-methyl-5-m-tolyl-thiazole-4- | | |
| | carbonyl)-thiazolidin-2-ylmethyl]-amide | | |
| 143 | rac-1,3-Dimethyl-1H-pyrazole-4-carboxylic acid [3- | 455.97 | 0.86 |
| | (2-methyl-5-m-tolyl-thiazole-4-carbonyl)-thiazolidin- | | |
| | 2-ylmethyl]-amide | | |
| 144 | rac-Benzo[1,2,3]thiadiazole-5-carboxylic acid [3-(2- | 495.80 | 0.97 |
| | methyl-5-m-tolyl-thiazole-4-carbonyl)-thiazolidin-2- | | |
| | ylmethyl]-amide | | |
| 145 | rac-Benzothiazole-6-carboxylic acid [3-(2-methyl-5- | 494.99 | 0.94 |
| | m-tolyl-thiazole-4-carbonyl)-thiazolidin-2-ylmethyl]- | | |
| | amide | | |
| 146 | rac-1,3,5-Trimethyl-1H-pyrazole-4-carboxylic acid | 470.06 | 0.87 |
| | [3-(2-methyl-5-m-tolyl-thiazole-4-carbonyl)- | | |
| | thiazolidin-2-ylmethyl]-amide | | |
| 147 | rac-2,2-Dimethyl-2,3-dihydro-benzofuran-7- | 508.22 | 1.05 |
| | carboxylic acid [3-(2-methyl-5-m-tolyl-thiazole-4- | | |
| | carbonyl)-thiazolidin-2-ylmethyl]-amide | | |
| 148 | rac-2,2-Difluoro-benzo[1,3]dioxole-4-carboxylic | 517.97 | 1.04 |
| | acid [3-(2-methyl-5-m-tolyl-thiazole-4-carbonyl)- | | |
| | thiazolidin-2-ylmethyl]-amide | | |
| 149 | rac-1-Methyl-1H-indole-5-carboxylic acid [3-(2- | 491.01 | 0.98 |
| | methyl-5-m-tolyl-thiazole-4-carbonyl)-thiazolidin-2- | | |
| | ylmethyl]-amide | | |
| 150 | rac-2,3-Dihydro-benzo[1,4]dioxine-6-carboxylic | 495.99 | 0.96 |
| | acid [3-(2-methyl-5-m-tolyl-thiazole-4-carbonyl)- | | |
| | thiazolidin-2-ylmethyl]-amide | | |
| 151 | rac-3H-Benzoimidazole-5-carboxylic acid [3-(2- | 478.02 | 0.74 |
| | methyl-5-m-tolyl-thiazole-4-carbonyl)-thiazolidin-2- | | |
| | ylmethyl]-amide | | |
| 152 | rac-1-Isopropyl-1H-pyrazole-4-carboxylic acid [3- | 469.94 | 0.89 |
| | (2-methyl-5-m-tolyl-thiazole-4-carbonyl)-thiazolidin- | | |
| | 2-ylmethyl]-amide | | |
| 153 | rac-2-Methyl-benzothiazole-5-carboxylic acid [3-(2- | 432.02 | 0.82 |
| | methyl-5-m-tolyl-thiazole-4-carbonyl)-thiazolidin-2- | | |
| | ylmethyl]-amide | | |
| 154 | rac-1-Methyl-1H-benzotriazole-5-carboxylic acid [3- | 492.99 | 0.90 |
| | (2-methyl-5-m-tolyl-thiazole-4-carbonyl)-thiazolidin- | | |
| | 2-ylmethyl]-amide | | |
| 155 | rac-1-Methyl-1H-benzoimidazole-5-carboxylic acid | 492.01 | 0.76 |
| | [3-(2-methyl-5-m-tolyl-thiazole-4-carbonyl)- | | |
| | thiazolidin-2-ylmethyl]-amide | | |
| 156 | rac-Quinoxaline-5-carboxylic acid [3-(2-methyl-5- | 490.31 | 0.99 |
| | m-tolyl-thiazole-4-carbonyl)-thiazolidin-2-ylmethyl]- | | |
| | amide | | |
| 157 | rac-1H-Indazole-4-carboxylic acid [3-(2-methyl-5- | 478.08 | 0.93 |
| | m-tolyl-thiazole-4-carbonyl)-thiazolidin-2-ylmethyl]- | | |
| | amide | | |
| 158 | rac-4-Chloro-pyridine-2-carboxylic acid [3-(2- | 472.94 | 0.99 |
| | methyl-5-m-tolyl-thiazole-4-carbonyl)-thiazolidin-2- | | |
| | ylmethyl]-amide | | |
| 159 | rac-4-Methoxy-pyridine-2-carboxylic acid [3-(2- | 468.98 | 0.90 |
| | methyl-5-m-tolyl-thiazole-4-carbonyl)-thiazolidin-2- | | |
| | ylmethyl]-amide | | |
| 160 | rac-2-Methoxy-N-[3-(2-methyl-5-m-tolyl-thiazole-4- | 468.99 | 0.95 |
| | carbonyl)-thiazolidin-2-ylmethyl]-nicotinamide | | |
| 161 | rac-6-Methyl-pyridine-2-carboxylic acid [3-(2- | 452.96 | 0.96 |
| | methyl-5-m-tolyl-thiazole-4-carbonyl)-thiazolidin-2- | | |
| | ylmethyl]-amide | | |
| 162 | rac-6-Trifluoromethyl-pyridine-2-carboxylic acid [3- | 506.66 | 1.03 |
| | (2-methyl-5-m-tolyl-thiazole-4-carbonyl)-thiazolidin- | | |
| | 2-ylmethyl]-amide | | |
| 163 | rac-2,5-Dimethyl-oxazole-4-carboxylic acid [3-(2- | 456.97 | 0.94 |
| | methyl-5-m-tolyl-thiazole-4-carbonyl)-thiazolidin-2- | | |
| | ylmethyl]-amide | | |
| 164 | rac-6-Methoxy-pyridine-2-carboxylic acid [3-(2- | 469.03 | 0.99 |
| | methyl-5-m-tolyl-thiazole-4-carbonyl)-thiazolidin-2- | | |
| | ylmethyl]-amide | | |
| 165 | rac-2,4-Dimethyl-thiazole-5-carboxylic acid [3-(2- | 472.83 | 0.91 |
| | methyl-5-m-tolyl-thiazole-4-carbonyl)-thiazolidin-2- | | |
| | ylmethyl]-amide | | |
| 166 | rac-2-Methyl-thiazole-4-carboxylic acid [3-(2- | 458.84 | 0.93 |
| | methyl-5-m-tolyl-thiazole-4-carbonyl)-thiazolidin-2- | | |
| | ylmethyl]-amide | | |
| 167 | rac-4-Methyl-thiazole-5-carboxylic acid [3-(2- | 458.73 | 0.90 |
| | methyl-5-m-tolyl-thiazole-4-carbonyl)-thiazolidin-2- | | |
| | ylmethyl]-amide | | |
| 168 | rac-3-Methyl-quinoxaline-2-carboxylic acid [3-(2- | 504.03 | 1.00 |
| | methyl-5-m-tolyl-thiazole-4-carbonyl)-thiazolidin-2- | | |
| | ylmethyl]-amide | | |
| 169 | rac-5-Chloro-pyridine-2-carboxylic acid [3-(2- | 472.88 | 1.00 |
| | methyl-5-m-tolyl-thiazole-4-carbonyl)-thiazolidin-2- | | |
| | ylmethyl]-amide | | |
| 170 | rac-5-Methyl-thiazole-2-carboxylic acid [3-(2- | 458.69 | 0.97 |
| | methyl-5-m-tolyl-thiazole-4-carbonyl)-thiazolidin-2- | | |
| | ylmethyl]-amide | | |
| 171 | rac-2,6-Dimethoxy-N-[3-(2-methyl-5-m-tolyl- | 498.78 | 1.01 |
| | thiazole-4-carbonyl)-thiazolidin-2-ylmethyl]- | | |
| | nicotinamide | | |
| 172 | rac-Thiazole-4-carboxylic acid [3-(2-methyl-5-m- | 444.97 | 0.90 |
| | tolyl-thiazole-4-carbonyl)-thiazolidin-2-ylmethyl]- | | |
| | amide | | |
| 173 | rac-2,4-Dimethyl-oxazole-5-carboxylic acid [3-(2- | 456.88 | 0.88 |
| | methyl-5-m-tolyl-thiazole-4-carbonyl)-thiazolidin-2- | | |
| | ylmethyl]-amide | | |
| 174 | rac-4-Methyl-thiazole-2-carboxylic acid [3-(2- | 458.81 | 0.97 |
| | methyl-5-m-tolyl-thiazole-4-carbonyl)-thiazolidin-2- | | |
| | ylmethyl]-amide | | |
| 175 | rac-6-Methyl-N-[3-(2-methyl-5-m-tolyl-thiazole-4- | 452.94 | 0.77 |
| | carbonyl)-thiazolidin-2-ylmethyl]-nicotinamide | | |
| 176 | rac-5-Methyl-N-[3-(2-methyl-5-m-tolyl-thiazole-4- | 453.03 | 0.80 |
| | carbonyl)-thiazolidin-2-ylmethyl]-nicotinamide | | |
| 177 | rac-4-Methyl-oxazole-5-carboxylic acid [3-(2- | 442.87 | 0.87 |
| | methyl-5-m-tolyl-thiazole-4-carbonyl)-thiazolidin-2- | | |
| | ylmethyl]-amide | | |
| 178 | rac-[2,6]Naphthyridine-3-carboxylic acid [3-(2- | 489.89 | 0.88 |
| | methyl-5-m-tolyl-thiazole-4-carbonyl)-thiazolidin-2- | | |
| | ylmethyl]-amide | | |
| 179 | rac-[1,5]Naphthyridine-2-carboxylic acid [3-(2- | 489.89 | 0.96 |
| | methyl-5-m-tolyl-thiazole-4-carbonyl)-thiazolidin-2- | | |
| | ylmethyl]-amide | | |
| 180 | rac-Quinoxaline-2-carboxylic acid [3-(2-methyl-5- | 489.92 | 0.99 |
| | m-tolyl-thiazole-4-carbonyl)-thiazolidin-2-ylmethyl]- | | |
| | amide | | |
| 181 | rac-5-Methyl-isoxazole-3-carboxylic acid [3-(2- | 442.88 | 0.94 |
| | methyl-5-m-tolyl-thiazole-4-carbonyl)-thiazolidin-2- | | |
| | ylmethyl]-amide | | |
| 182 | rac-[1,8]Naphthyridine-2-carboxylic acid [3-(2- | 489.93 | 0.87 |
| | methyl-5-m-tolyl-thiazole-4-carbonyl)-thiazolidin-2- | | |
| | ylmethyl]-amide | | |
| 183 | rac-4-Methyl-N-[3-(2-methyl-5-m-tolyl-thiazole-4- | 453 | 0.77 |
| | carbonyl)-thiazolidin-2-ylmethyl]-nicotinamide | | |
| 184 | rac-N-[3-(2-Methyl-5-m-tolyl-thiazole-4-carbonyl)- | 506.70 | 0.99 |
| | thiazolidin-2-ylmethyl]-6-trifluoromethyl- | | |
| | nicotinamide | | |
| 185 | rac-1H-Pyrazolo[3,4-b]pyridine-5-carboxylic acid | 478.07 | 0.87 |
| | [3-(2-methyl-5-m-tolyl-thiazole-4-carbonyl)- | | |
| | thiazolidin-2-ylmethyl]-amide | | |
| 186 | rac-Oxazole-4-carboxylic acid [3-(2-methyl-5-m- | 428.98 | 0.87 |
| | tolyl-thiazole-4-carbonyl)-thiazolidin-2-ylmethyl]- | | |
| | amide | | |
| 187 | rac-N-[3-(2-Methyl-5-m-tolyl-thiazole-4-carbonyl)- | 506.07 | 0.94 |
| | thiazolidin-2-ylmethyl]-4-trifluoromethyl- | | |
| | nicotinamide | | |
| 188 | rac-1H-Pyrazolo[3,2-b]pyridine-6-carboxylic acid | 477.96 | 0.74 |
| | [3-(2-methyl-5-m-tolyl-thiazole-4-carbonyl)- | | |
| | thiazolidin-2-ylmethyl]-amide | | |
| 189 | rac-4-Chloro-N-[3-(2-methyl-5-m-tolyl-thiazole-4- | 472.85 | 0.89 |
| | carbonyl)-thiazolidin-2-ylmethyl]-nicotinamide | | |
| 190 | rac-Benzothiazole-7-carboxylic acid {3-[2- | 539.98 | 0.95 |
| | dimethylamino-5-(3-methoxy-phenyl)-thiazole-4- | | |
| | carbonyl]-thiazolidin-2-ylmethyl}-amide | | |
| 191 | rac-Benzothiazole-7-carboxylic acid {3-[2- | 527.98 | 0.96 |
| | dimethylamino-5-(4-fluoro-phenyl)-thiazole-4- | | |
| | carbonyl]-thiazolidin-2-ylmethyl}-amide | | |
| 192 | rac-Benzothiazole-7-carboxylic acid {3-[2- | 528.00 | 0.97 |
| | dimethylamino-5-(3-fluoro-phenyl)-thiazole-4- | | |
| | carbonyl]-thiazolidin-2-ylmethyl}-amide | | |
| 193 | rac-Benzothiazole-7-carboxylic acid {3-[2- | 537.89 | 1.01 |
| | dimethylamino-5-(3,4-dimethyl-phenyl)-thiazole-4- | | |
| | carbonyl]-thiazolidin-2-ylmethyl}-amide | | |
| 194 | rac-Benzothiazole-7-carboxylic acid [3-(3-m-tolyl- | 475.98 | 0.94 |
| | pyrazine-2-carbonyl)-thiazolidin-2-ylmethyl]-amide | | |
| 195 | rac-Benzothiazole-7-carboxylic acid {3-[3-(3,4- | 489.89 | 0.97 |
| | dimethyl-phenyl)-pyrazine-2-carbonyl]-thiazolidin-2- | | |
| | ylmethyl}-amide | | |
| 196 | rac-Benzothiazole-7-carboxylic acid {3-[3-(3- | 492 | 0.91 |
| | methoxy-phenyl)-pyrazine-2-carbonyl]-thiazolidin- | | |
| | 2-ylmethyl}-amide | | |
| 197 | rac-2,3-Dihydro-benzofuran-4-carboxylic acid {3-[5- | 485.77 | 1.0 |
| | (3-chloro-phenyl)-thiazole-4-carbonyl]-thiazolidin-2- | | |
| | ylmethyl}-amide | | |
| 198 | rac-2,3-Dihydro-benzofuran-4-carboxylic acid {3-[5- | 479.86 | 1.02 |
| | (3,4-dimethyl-phenyl)-thiazole-4-carbonyl]- | | |
| | thiazolidin-2-ylmethyl}-amide | | |
| 199 | rac-2,3-Dihydro-benzofuran-4-carboxylic acid {3-[2- | 513.4 | 1.02 |
| | dimethylamino-5-(4-fluoro-phenyl)-thiazole-4- | | |
| | carbonyl]-thiazolidin-2-ylmethyl}-amide | | |
| 200 | rac-2,3-Dihydro-benzofuran-4-carboxylic acid {3-[2- | 513.4 | 1.02 |
| | dimethylamino-5-(2-fluoro-phenyl)-thiazole-4- | | |
| | carbonyl]-thiazolidin-2-ylmethyl}-amide | | |
| 201 | rac-2,3-Dihydro-benzofuran-4-carboxylic acid {3-[2- | 539.16 | 1.04 |
| | (ethyl-methyl-amino)-5-(3-methoxy-phenyl)- | | |
| | thiazole-4-carbonyl]-thiazolidin-2-ylmethyl}-amide | | |
| 202 | rac-2,3-Dihydro-benzofuran-4-carboxylic acid {3-[2- | 527.8 | 1.05 |
| | (ethyl-methyl-amino)-5-(4-fluoro-phenyl)-thiazole-4- | | |
| | carbonyl]-thiazolidin-2-ylmethyl}-amide | | |
| 203 | rac-2,3-Dihydro-benzofuran-4-carboxylic acid {3-[2- | 525.16 | 1.01 |
| | dimethylamino-5-(3-methoxy-phenyl)-thiazole-4- | | |
| | carbonyl]-thiazolidin-2-ylmethyl}-amide | | |
| 204 | rac-2,3-Dihydro-benzofuran-4-carboxylic acid {3-[5- | 482.45 | 0.97 |
| | (3-methoxy-phenyl)-thiazole-4-carbonyl]- | | |
| | thiazolidin-2-ylmethyl}-amide | | |
| 205 | rac-2,3-Dihydro-benzofuran-4-carboxylic acid [3-(5- | 466.39 | 1.0 |
| | m-tolyl-thiazole-4-carbonyl)-thiazolidin-2-ylmethyl]- | | |
| | amide | | |
| 206 | rac-2,3-Dihydro-benzofuran-4-carboxylic acid [3-(2- | 508.95 | 1.03 |
| | dimethylamino-5-p-tolyl-thiazole-4-carbonyl)- | | |
| | thiazolidin-2-ylmethyl]-amide | | |
| 207 | rac-2,3-Dihydro-benzofuran-4-carboxylic acid {3-[2- | 527.37 | 1.05 |
| | dimethylamino-5-(3-fluoro-4-methyl-phenyl)- | | |
| | thiazole-4-carbonyl]-thiazolidin-2-ylmethyl}-amide | | |
| 208 | rac-2,3-Dihydro-benzofuran-4-carboxylic acid {3-[5- | 528.94 | 1.05 |
| | (3-chloro-phenyl)-2-dimethylamino-thiazole-4- | | |
| | carbonyl]-thiazolidin-2-ylmethyl}-amide | | |
| 209 | rac-2,3-Dihydro-benzofuran-4-carboxylic acid {3-[3- | 474.89 | 0.99 |
| | (3,4-dimethyl-phenyl)-pyrazine-2-carbonyl]- | | |
| | thiazolidin-2-ylmethyl}-amide | | |
| 210 | rac-2,3-Dihydro-benzofuran-4-carboxylic acid {3-[3- | 478.89 | 0.98 |
| | (4-fluoro-3-methyl-phenyl)-pyrazine-2-carbonyl]- | | |
| | thiazolidin-2-ylmethyl}-amide | | |
| 211 | rac-Benzothiazole-7-carboxylic acid {3-[2- | 528.05 | 0.98 |
| | dimethylamino-5-(4-fluoro-phenyl)-thiazole-4- | | |
| | carbonyl]-thiazolidin-2-ylmethyl}-amide | | |
| 212 | rac-Benzothiazole-7-carboxylic acid {3-[2- | 540.07 | 0.97 |
| | dimethylamino-5-(3-methoxy-phenyl)-thiazole-4- | | |
| | carbonyl]-thiazolidin-2-ylmethyl}-amide | | |
| 213 | rac-Benzothiazole-7-carboxylic acid [3-(2- | 524.21 | 1.0 |
| | dimethylamino-5-p-tolyl-thiazole-4-carbonyl)- | | |
| | thiazolidin-2-ylmethyl]-amide | | |
| 214 | rac-Benzothiazole-7-carboxylic acid {3-[2- | 541.88 | 1.02 |
| | dimethylamino-5-(3-fluoro-4-methyl-phenyl)- | | |
| | thiazole-4-carbonyl]-thiazolidin-2-ylmethyl}-amide | | |
| 215 | rac-1-Methyl-1H-indole-3-carboxylic acid {3-[5-(4- | 480.81 | 0.97 |
| | fluoro-phenyl)-thiazole-4-carbonyl]-thiazolidin-2- | | |
| | ylmethyl}-amide | | |
| 216 | rac-1-Methyl-5-trifluoromethyl-1H-pyrazole-4- | 500.16 | 0.94 |
| | carboxylic acid {3-[5-(4-fluoro-phenyl)-thiazole-4- | | |
| | carbonyl]-thiazolidin-2-ylmethyl}-amide | | |
| 217 | rac-1-Methyl-1H-indole-3-carboxylic acid {3-[5-(3- | 480.27 | 0.98 |
| | fluoro-phenyl)-thiazole-4-carbonyl]-thiazolidin-2- | | |
| | ylmethyl}-amide | | |
| 218 | rac-1-Methyl-5-trifluoromethyl-1H-pyrazole-4- | 500.11 | 0.94 |
| | carboxylic acid {3-[5-(3-fluoro-phenyl)-thiazole-4- | | |
| | carbonyl]-thiazolidin-2-ylmethyl}-amide | | |
| 219 | rac-1-Ethyl-3-methyl-1H-pyrazole-4-carboxylic acid | 460.17 | 0.88 |
| | {3-[5-(3-fluoro-phenyl)-thiazole-4-carbonyl]- | | |
| | thiazolidin-2-ylmethyl}-amide | | |
| 220 | rac-1,3-Dimethyl-1H-pyrazole-4-carboxylic acid {3- | 446.11 | 0.85 |
| | [5-(3-fluoro-phenyl)-thiazole-4-carbonyl]-thiazolidin- | | |
| | 2-ylmethyl}-amide | | |
| 221 | rac-Quinoxaline-5-carboxylic acid {3-[5-(3-fluoro- | 479.82 | 0.95 |
| | phenyl)-thiazole-4-carbonyl]-thiazolidin-2-ylmethyl}- | | |
| | amide | | |

### II—Biological Assays

### In Vitro Assay

The orexin receptor antagonistic activity of the compounds of formula (I) is determined in accordance with the following experimental method.

### Experimental Method:

Chinese hamster ovary (CHO) cells expressing the human orexin-1 receptor and the human orexin-2 receptor, respectively, are grown in culture medium (Ham F-12 with L-Glutamine) containing 300 μg/ml G418, 100 μml penicillin, 100 μg/ml streptomycin and 10% heat inactivated fetal calf serum (FCS). The cells are seeded at 20′000 cells/well into 384-well black clear bottom sterile plates (Greiner). The seeded plates are incubated overnight at 37° C. in 5% CO₂.

Human orexin-A as an agonist is prepared as 1 mM stock solution in MeOH:water (1:1), diluted in HBSS containing 0.1% bovine serum albumin (BSA), NaHCO₃: 0.375 g/l and 20 mM HEPES for use in the assay at a final concentration of 3 nM.

Antagonists are prepared as 10 mM stock solution in DMSO, then diluted in 384-well plates using DMSO followed by a transfer of the dilutions into in HBSS containing 0.1% bovine serum albumin (BSA), NaHCO₃: 0.375 g/l and 20 mM HEPES. On the day of the assay, 50 μl of staining buffer (HBSS containing 1% FCS, 20 mM HEPES, NaHCO₃: 0.375 g/l, 5 mM probenecid (Sigma) and 3 μM of the fluorescent calcium indicator fluo-4 AM (1 mM stock solution in DMSO, containing 10% pluronic) is added to each well. The 384-well cell-plates are incubated for 50 min at 37° C. in 5% CO₂ followed by equilibration at rt for 30-120 min before measurement.

Within the Fluorescent Imaging Plate Reader (FLIPR Tetra, Molecular Devices), antagonists are added to the plate in a volume of 10 μl/well, incubated for 10 min and finally 10 it/well of agonist is added. Fluorescence is measured for each well at 1 second intervals, and the height of each fluorescence peak is compared to the height of the fluorescence peak induced by 3 nM orexin-A with vehicle in place of antagonist. For each antagonist, the IC₅₀ value (the concentration of compound needed to inhibit 50% of the agonistic response) is determined and may be normalized using the obtained IC₅₀ value of a on-plate reference compound (normalized values in Table 1 are indicated by an asterisk *). Optimized conditions were achieved by adjustment of pipetting speed and cell splitting regime. The calculated IC₅₀ values of the compounds may fluctuate depending on the daily cellular assay performance. Fluctuations of this kind are known to those skilled in the art.

Antagonistic activities (IC₅₀ values) of all exemplified compounds are in the range of 1-503 nM with an average of 23 nM with respect to the OX1 receptor. IC₅₀ values of all exemplified compounds are in the range of 1-3099 nM with an average of 74 nM with respect to the OX2 receptor. Antagonistic activities of selected compounds are displayed in Table 1.

TABLE 1

| Compound of Example | OX₁ IC₅₀ (nM) | OX₂ IC₅₀ (nM) |
|---|---|---|
| 4 | 2 | 2 |
| 11 | 17 | 63 |
| 17 | 4*² | 6*² |
| 29 | 3 | 7 |
| 32 | 4 | 5 |
| 39 | 8 | 86 |
| 42 | 10 | 31 |
| 50 | 7 | 43 |
| 57 | 4 | 10 |
| 61 | 16 | 28 |
| 72 | 12 | 10 |
| 77 | 4*³ | 4*³ |
| 83 | 14 | 11 |
| 88 | 4*³ | 7*³ |
| 95 | 23 | 68 |
| 104 | 7 | 8 |
| 109 | 7 | 10 |
| 116 | 2 | 5 |
| 120 | 8*² | 7*² |
| 126 | 11 | 11 |
| 128 | 12 | 14 |
| 140 | 9* | 9* |
| 142 | 5* | 12* |
| 160 | 4* | 15* |
| 162 | 5* | 41* |
| 163 | 5* | 8* |
| 165 | 6* | 5* |
| 196 | 3* | 3* |

*IC₅₀value normalized as described above
*²geometric mean of n = 2 values
*³geometric mean of n = 3 values

1. A compound of formula (I) 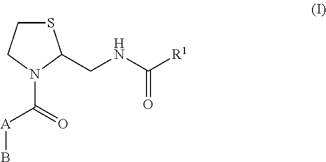
wherein
A represents aryl or heteroaryl, wherein the aryl or heteroaryl is independently unsubstituted or mono- or di-substituted, wherein the substituents are independently selected from the group consisting of (C₁₋₄)alkyl, (C₃₋₆)cycloalkyl, (C₁₋₄)alkoxy, trifluoromethyl, —NR²R³ and halogen;
B represents aryl or heteroaryl, wherein the aryl or heteroaryl is independently unsubstituted or mono-, di-, or tri-substituted, wherein the substituents are independently selected from the group consisting of (C₁₋₄)alkyl, (C₁₋₄)alkoxy, fluoroalkyl, fluoroalkoxy, cyano, and halogen;
R¹ represents aryl or heteroaryl, wherein the aryl or heteroaryl is independently unsubstituted or mono-, di-, or tri-substituted, wherein the substituents are independently selected from the group consisting of (C₁₋₄)alkyl, (C₁₋₄)alkoxy, halogen, cyano, fluoroalkyl, fluoroalkoxy, and —NR²R³; or
R¹ represents heterocyclyl wherein said heterocyclyl is unsubstituted or mono- or di-substituted wherein the substituents are independently selected from the group consisting of (C₁₋₄)alkyl, (C₁₋₄)alkoxy, halogen, and oxo;
R² represents hydrogen or (C₁₋₄)alkyl; and
R³ represents hydrogen or (C₁₋₄)alkyl;
or a salt thereof.

2. A compound according to claim 1, wherein A represents a 5- to 6-membered monocyclic heteroaryl which is unsubstituted or mono-substituted, wherein the substituent is selected from the group consisting of (C₁₋₄)alkyl, (C₃₋₆)cycloalkyl, and —NR²R³;
or a salt thereof.

3. A compound according to claim 1, wherein A represents a group selected from the group consisting thiophen-2-yl, thiophen-3-yl, 2-methyl-oxazol-4-yl, 2-methyl-thiazol-5-yl, thiazol-4-yl, 2-methyl-thiazol-4-yl, 2-amino-thiazol-4-yl, 2-dimethylamino-thiazol-4-yl, 2-bromo-thiazol-4-yl, 2-methoxy-thiazol-4-yl, 2-cyclopropyl-thiazol-4-yl, and pyrazin-2-yl;
or a salt thereof.

4. A compound according to claim 1, wherein B represents aryl which is unsubstituted or mono-, di-, or tri-substituted, wherein the substituents are independently selected from the group consisting of (C₁₋₄)alkyl, (C₁₋₄)alkoxy, fluoroalkyl, fluoroalkoxy, and halogen;
or a salt thereof.

5. A compound according to claim 1, wherein R¹ represents heteroaryl, which is unsubstituted or mono- or di-substituted wherein the substituents are independently selected from the group consisting of (C₁₋₄)alkyl, (C₁₋₄)alkoxy, trifluoromethyl, and halogen; or R¹ represents heterocyclyl wherein said heterocyclyl is unsubstituted or mono- or di-substituted wherein the substituents are independently selected from the group consisting of (C₁₋₄)alkyl, halogen and oxo;
or a salt thereof.

6. A compound according to claim 1, wherein, in case R¹ represents heteroaryl, said heteroaryl is selected from the group consisting of isoxazolyl, pyrazolyl, pyridyl, pyrimidyl, indolyl, benzofuranyl, benzothiophenyl, indazolyl, benzimidazolyl, benzoxazolyl, benzisoxazolyl, benzothiazolyl, benzoisothiazolyl benzotriazolyl, benzo[2,1,3]oxadiazolyl, benzo[2,1,3]thiadiazolyl, benzo[1,2,3]thiadiazolyl, quinolinyl, isoquinolinyl, quinoxalinyl, pyrazolo[1,5-a]pyridyl, imidazo[1,2-a]pyridyl, 1H-pyrrolo[3,2-b]pyridyl, 1H-pyrrolo[2,3-b]pyridyl, 4H-furo[3,2-b]pyrrolyl, pyrrolo[2,1-b]thiazolyl, imidazo[2,1-b]thiazolyl, oxazolyl, and thiazolyl; wherein said heteroaryl is unsubstituted or mono- or di-substituted wherein the substituents are independently selected from the group consisting of (C₁₋₄)alkyl, (C₁₋₄)alkoxy, trifluoromethyl, and halogen;
or a salt thereof.

7. A compound according to claim 1, wherein, in case R¹ represents heterocyclyl, said heterocyclyl is selected from the group consisting of 2,3-dihydro-benzofuranyl, 4H-benzo[1,3]dioxinyl, benzo[1,3]dioxolyl, 3,4-dihydro-2H-benzo[1,4]oxazinyl, 2,3-dihydro-benzo[1,4]dioxinyl, 2H-chromenyl, and chromanyl, wherein said heterocyclyl is unsubstituted or mono- or di-substituted wherein the substituents are independently selected from the group consisting of (C₁₋₄)alkyl, halogen and oxo;
or a salt thereof.

8. A compound according to claim 1 selected from the group consisting of:
2-Methyl-benzofuran-4-carboxylic acid [3-(2-methyl-5-m-tolyl-thiazole-4-carbonyl)-thiazolidin-2-ylmethyl]-amide;
5-Chloro-2-methyl-benzofuran-4-carboxylic acid [3-(2-methyl-5-m-tolyl-thiazole-4-carbonyl)-thiazolidin-2-ylmethyl]-amide;
2-Methyl-benzooxazole-7-carboxylic acid [3-(2-methyl-5-m-tolyl-thiazole-4-carbonyl)-thiazolidin-2-ylmethyl]-amide;
Pyrrolo[2,1-b]thiazole-7-carboxylic acid [3-(2-methyl-5-m-tolyl-thiazole-4-carbonyl)-thiazolidin-2-ylmethyl]-amide;
Benzo[d]isoxazole-3-carboxylic acid [3-(2-methyl-5-m-tolyl-thiazole-4-carbonyl)-thiazolidin-2-ylmethyl]-amide;
7-Chloro-2-methyl-benzofuran-4-carboxylic acid [3-(2-methyl-5-m-tolyl-thiazole-4-carbonyl)-thiazolidin-2-ylmethyl]-amide;
3-Methyl-benzofuran-4-carboxylic acid [3-(2-methyl-5-m-tolyl-thiazole-4-carbonyl)-thiazolidin-2-ylmethyl]-amide;
2-Methyl-benzooxazole-4-carboxylic acid [3-(2-methyl-5-m-tolyl-thiazole-4-carbonyl)-thiazolidin-2-ylmethyl]-amide;
6-Methyl-pyrrolo[2,1-b]thiazole-7-carboxylic acid [3-(2-methyl-5-m-tolyl-thiazole-4-carbonyl)-thiazolidin-2-ylmethyl]-amide;
6-Methyl-imidazo[2,1-b]thiazole-5-carboxylic acid [3-(2-methyl-5-m-tolyl-thiazole-4-carbonyl)-thiazolidin-2-ylmethyl]-amide;
2-Methyl-7-trifluoromethyl-benzofuran-4-carboxylic acid [3-(2-methyl-5-m-tolyl-thiazole-4-carbonyl)-thiazolidin-2-ylmethyl]-amide;
2,3-Dimethyl-benzofuran-4-carboxylic acid [3-(2-methyl-5-m-tolyl-thiazole-4-carbonyl)-thiazolidin-2-ylmethyl]-amide;
Imidazo[1,2-a]pyridine-3-carboxylic acid [3-(2-methyl-5-m-tolyl-thiazole-4-carbonyl)-thiazolidin-2-ylmethyl]-amide;
Imidazo[2,1-b]thiazole-5-carboxylic acid [3-(2-methyl-5-m-tolyl-thiazole-4-carbonyl)-thiazolidin-2-ylmethyl]-amide;
7-Fluoro-2-methyl-benzofuran-4-carboxylic acid [3-(2-methyl-5-m-tolyl-thiazole-4-carbonyl)-thiazolidin-2-ylmethyl]-amide;
2,3-Dihydro-benzofuran-4-carboxylic acid [3-(2-methyl-5-m-tolyl-thiazole-4-carbonyl)-thiazolidin-2-ylmethyl]-amide;
Benzooxazole-4-carboxylic acid [3-(2-methyl-5-m-tolyl-thiazole-4-carbonyl)-thiazolidin-2-ylmethyl]-amide;
2-Methyl-imidazo[1,2-a]pyridine-3-carboxylic acid [3-(2-methyl-5-m-tolyl-thiazole-4-carbonyl)-thiazolidin-2-ylmethyl]-amide;
2-Methyl-6-trifluoromethyl-benzofuran-4-carboxylic acid [3-(2-methyl-5-m-tolyl-thiazole-4-carbonyl)-thiazolidin-2-ylmethyl]-amide;
6-Fluoro-2-methyl-benzofuran-4-carboxylic acid [3-(2-methyl-5-m-tolyl-thiazole-4-carbonyl)-thiazolidin-2-ylmethyl]-amide;
2,3-Dihydro-benzo[1,4]dioxine-5-carboxylic acid [3-(2-methyl-5-m-tolyl-thiazole-4-carbonyl)-thiazolidin-2-ylmethyl]-amide;
Benzo[1,2,5]oxadiazole-4-carboxylic acid [3-(2-methyl-5-m-tolyl-thiazole-4-carbonyl)-thiazolidin-2-ylmethyl]-amide;
1-Methyl-1H-indazole-3-carboxylic acid [3-(2-methyl-5-m-tolyl-thiazole-4-carbonyl)-thiazolidin-2-ylmethyl]-amide;
6-Chloro-2-methyl-benzofuran-4-carboxylic acid [3-(2-methyl-5-m-tolyl-thiazole-4-carbonyl)-thiazolidin-2-ylmethyl]-amide;
Benzooxazole-7-carboxylic acid [3-(2-methyl-5-m-tolyl-thiazole-4-carbonyl)-thiazolidin-2-ylmethyl]-amide;
Benzo[1,2,5]thiadiazole-4-carboxylic acid [3-(2-methyl-5-m-tolyl-thiazole-4-carbonyl)-thiazolidin-2-ylmethyl]-amide;
Benzo[d]isothiazole-3-carboxylic acid [3-(2-methyl-5-m-tolyl-thiazole-4-carbonyl)-thiazolidin-2-ylmethyl]-amide;
Benzothiazole-7-carboxylic acid [3-(2-methyl-5-m-tolyl-thiazole-4-carbonyl)-thiazolidin-2-ylmethyl]-amide;
2,3-Dihydro-benzofuran-4-carboxylic acid {3-[4-(3-chloro-phenyl)-2-methyl-thiazole-5-carbonyl]-thiazolidin-2-ylmethyl}-amide;
1-Methyl-1H-indazole-3-carboxylic acid {3-[4-(3-chloro-phenyl)-2-methyl-thiazole-5-carbonyl]-thiazolidin-2-ylmethyl}-amide;
Benzothiazole-7-carboxylic acid {3-[4-(3-methoxy-phenyl)-2-methyl-thiazole-5-carbonyl]-thiazolidin-2-ylmethyl}-amide;
2,3-Dihydro-benzofuran-4-carboxylic acid {3-[4-(3-methoxy-phenyl)-2-methyl-thiazole-5-carbonyl]-thiazolidin-2-ylmethyl}-amide;
1-Methyl-1H-indazole-3-carboxylic acid {3-[4-(3-methoxy-phenyl)-2-methyl-thiazole-5-carbonyl]-thiazolidin-2-ylmethyl}-amide;
Benzothiazole-7-carboxylic acid {3-[4-(3-chloro-phenyl)-2-methyl-thiazole-5-carbonyl]-thiazolidin-2-ylmethyl}-amide;
Imidazo[1,2-a]pyridine-3-carboxylic acid {3-[4-(3-chloro-phenyl)-2-methyl-thiazole-5-carbonyl]-thiazolidin-2-ylmethyl}-amide;
2,3-Dihydro-benzofuran-4-carboxylic acid {3-[4-(4-fluoro-phenyl)-2-methyl-thiazole-5-carbonyl]-thiazolidin-2-ylmethyl}-amide;
2,3-Dihydro-benzofuran-4-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid {3-[4-(3-chloro-phenyl)-2-methyl-thiazole-5-carbonyl]-thiazolidin-2-ylmethyl}-amide;
1-Methyl-1H-indazole-3-carboxylic acid {3-[4-(4-fluoro-phenyl)-2-methyl-thiazole-5-carbonyl]-thiazolidin-2-ylmethyl}-amide;
Benzothiazole-7-carboxylic acid {3-[4-(4-fluoro-phenyl)-2-methyl-thiazole-5-carbonyl]-thiazolidin-2-ylmethyl}-amide;
1-Methyl-1H-indazole-3-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid [3-(2-methyl-4-p-tolyl-thiazole-5-carbonyl)-thiazolidin-2-ylmethyl]-amide;
2,3-Dihydro-benzo[1,4]dioxine-5-carboxylic acid {3-[4-(3-methoxy-phenyl)-2-methyl-thiazole-5-carbonyl]-thiazolidin-2-ylmethyl}-amide;
2,3-Dihydro-benzo[1,4]dioxine-5-carboxylic acid {3-[4-(4-fluoro-phenyl)-2-methyl-thiazole-5-carbonyl]-thiazolidin-2-ylmethyl}-amide;
Imidazo[1,2-a]pyridine-3-carboxylic acid {3-[4-(3-methoxy-phenyl)-2-methyl-thiazole-5-carbonyl]-thiazolidin-2-ylmethyl}-amide;
Imidazo[1,2-a]pyridine-3-carboxylic acid {3-[4-(4-fluoro-phenyl)-2-methyl-thiazole-5-carbonyl]-thiazolidin-2-ylmethyl}-amide;
Imidazo[1,2-a]pyridine-3-carboxylic acid [3-(2-methyl-4-p-tolyl-thiazole-5-carbonyl)-thiazolidin-2-ylmethyl]-amide;
Benzothiazole-7-carboxylic acid {3-[2-methyl-4-(3-trifluoromethyl-phenyl)-thiazole-5-carbonyl]-thiazolidin-2-ylmethyl}-amide;
2,3-Dihydro-benzo[1,4]dioxine-5-carboxylic acid {3-[2-methyl-4-(3-trifluoromethyl-phenyl)-thiazole-5-carbonyl]-thiazolidin-2-ylmethyl}-amide;
1-Methyl-1H-indazole-3-carboxylic acid {3-[2-methyl-4-(3-trifluoromethyl-phenyl)-thiazole-5-carbonyl}-thiazolidin-2-ylmethyl]-amide;
Imidazo[1,2-a]pyridine-3-carboxylic acid {3-[2-methyl-4-(3-trifluoromethyl-phenyl)-thiazole-5-carbonyl]-thiazolidin-2-ylmethyl}-amide;
2,3-Dihydro-benzofuran-4-carboxylic acid [3-(2-methyl-5-phenyl-thiazole-4-carbonyl)-thiazolidin-2-ylmethyl]-amide;
2,3-Dihydro-benzo[1,4]dioxine-5-carboxylic acid {3-[5-(3-methoxy-phenyl)-2-methyl-thiazole-4-carbonyl]-thiazolidin-2-ylmethyl}-amide;
Benzothiazole-7-carboxylic acid {3-[5-(3-methoxy-phenyl)-2-methyl-thiazole-4-carbonyl]-thiazolidin-2-ylmethyl}-amide;
2,3-Dihydro-benzofuran-4-carboxylic acid {3-[5-(3-methoxy-phenyl)-2-methyl-thiazole-4-carbonyl]-thiazolidin-2-ylmethyl}-amide;
Imidazo[1,2-a]pyridine-3-carboxylic acid {3-[5-(3-methoxy-phenyl)-2-methyl-thiazole-4-carbonyl]-thiazolidin-2-ylmethyl}-amide;
2,3-Dihydro-benzo[1,4]dioxine-5-carboxylic acid [3-(2-methyl-5-phenyl-thiazole-4-carbonyl)-thiazolidin-2-ylmethyl]-amide;
2,3-Dihydro-benzofuran-4-carboxylic acid {3-[5-(3-chloro-phenyl)-2-methyl-thiazole-4-carbonyl]-thiazolidin-2-ylmethyl}-amide;
2,3-Dihydro-benzofuran-4-carboxylic acid {3-[2-methyl-5-(4-trifluoromethyl-phenyl)-thiazole-4-carbonyl]-thiazolidin-2-ylmethyl}-amide;
2,3-Dihydro-benzo[1,4]dioxine-5-carboxylic acid {3-[2-methyl-5-(4-trifluoromethyl-phenyl)-thiazole-4-carbonyl]-thiazolidin-2-ylmethyl}-amide;
1-Methyl-1H-indazole-3-carboxylic acid {3-[2-methyl-5-(4-trifluoromethyl-phenyl)-thiazole-4-carbonyl]-thiazolidin-2-ylmethyl}-amide;
Imidazo[1,2-a]pyridine-3-carboxylic acid {3-[2-methyl-5-(4-trifluoromethyl-phenyl)-thiazole-4-carbonyl]-thiazolidin-2-ylmethyl}-amide;
Chroman-8-carboxylic acid [3-(2-methyl-5-m-tolyl-thiazole-4-carbonyl)-thiazolidin-2-ylmethyl]-amide;
Chroman-5-carboxylic acid [3-(2-methyl-5-m-tolyl-thiazole-4-carbonyl)-thiazolidin-2-ylmethyl]-amide;
3,4-Dihydro-2H-benzo[1,4]oxazine-5-carboxylic acid [3-(2-methyl-5-m-tolyl-thiazole-4-carbonyl)-thiazolidin-2-ylmethyl]-amide;
3,4-Dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3,4-dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3,4-dihydro-2H-benzo[1,4]oxazine-5-carboxylic acid [3-(2-methyl-5-m-tolyl-thiazole-4-carbonyl)-thiazolidin-2-ylmethyl]-amide;
3-Oxo-3,4-dihydro-2H-benzo[1,4]oxazine-8-carboxylic acid [3-(2-methyl-5-m-tolyl-thiazole-4-carbonyl)-thiazolidin-2-ylmethyl]-amide;
4-Methyl-3-oxo-3,4-dihydro-2H-benzo[1,4]oxazine-8-carboxylic acid [3-(2-methyl-5-m-tolyl-thiazole-4-carbonyl)-thiazolidin-2-ylmethyl]-amide;
2,3-Dihydro-benzofuran-4-carboxylic acid [3-(3′,4′-dimethyl-biphenyl-2-carbonyl)-thiazolidin-2-ylmethyl]-amide;
Benzothiazole-7-carboxylic acid [3-(3′,4′-dimethyl-biphenyl-2-carbonyl)-thiazolidin-2-ylmethyl]-amide;
2,3-Dihydro-benzo[1,4]dioxine-5-carboxylic acid [3-(3′,4′-dimethyl-biphenyl-2-carbonyl)-thiazolidin-2-ylmethyl]-amide;
2,3-Dihydro-benzofuran-4-carboxylic acid {3-[2-methyl-4-(3-trifluoromethyl-phenyl)-thiazole-5-carbonyl]-thiazolidin-2-ylmethyl}-amide;
2,3-Dihydro-benzo[1,4]dioxine-5-carboxylic acid {3-[5-(4-fluoro-phenyl)-2-methyl-thiazole-4-carbonyl]-thiazolidin-2-ylmethyl}-amide;
Imidazo[1,2-a]pyridine-3-carboxylic acid {3-[5-(4-fluoro-phenyl)-2-methyl-thiazole-4-carbonyl]-thiazolidin-2-ylmethyl}-amide;
2,3-Dihydro-benzofuran-4-carboxylic acid (3-[5-(4-fluoro-phenyl)-2-methyl-thiazole-4-carbonyl]-thiazolidin-2-ylmethyl)-amide;
Benzothiazole-7-carboxylic acid {3-[5-(4-fluoro-phenyl)-2-methyl-thiazole-4-carbonyl]-thiazolidin-2-ylmethyl}-amide;
1-Methyl-1H-indazole-3-carboxylic acid [3-(2-methyl-5-phenyl-thiazole-4-carbonyl)-thiazolidin-2-ylmethyl]-amide;
2,3-Dihydro-benzofuran-4-carboxylic acid [3-(2-methyl-5-m-tolyl-oxazole-4-carbonyl)-thiazolidin-2-ylmethyl]-amide;
2,3-Dihydro-benzo[1,4]dioxine-5-carboxylic acid [3-(2-methyl-5-m-tolyl-oxazole-4-carbonyl)-thiazolidin-2-ylmethyl]-amide;
1-Methyl-1H-indazole-3-carboxylic acid [3-(3′,4′-dimethyl-biphenyl-2-carbonyl)-thiazolidin-2-ylmethyl]-amide;
Benzothiazole-7-carboxylic acid [3-(2-methyl-5-m-tolyl-oxazole-4-carbonyl)-thiazolidin-2-ylmethyl]-amide;
Imidazo[1,2-a]pyridine-3-carboxylic acid [3-(2-methyl-5-m-tolyl-oxazole-4-carbonyl)-thiazolidin-2-ylmethyl]-amide;
2,3-Dihydro-benzofuran-4-carboxylic acid {3-[2-methyl-5-(3-trifluoromethoxy-phenyl)-oxazole-4-carbonyl]-thiazolidin-2-ylmethyl}-amide;
2,3-Dihydro-benzo[1,4]dioxine-5-carboxylic acid {3-[2-methyl-5-(3-trifluoromethoxy-phenyl)-oxazole-4-carbonyl]-thiazolidin-2-ylmethyl}-amide;
1-Methyl-1H-indazole-3-carboxylic acid {3-[2-methyl-5-(3-trifluoromethoxy-phenyl)-oxazole-4-carbonyl]-thiazolidin-2-ylmethyl}-amide;
Benzothiazole-7-carboxylic acid [3-(2-methyl-5-phenyl-oxazole-4-carbonyl)-thiazolidin-2-ylmethyl]-amide;
1-Methyl-1H-indazole-3-carboxylic acid [3-(2-methyl-5-phenyl-oxazole-4-carbonyl)-thiazolidin-2-ylmethyl]-amide;
2,3-Dihydro-benzo[1,4]dioxine-5-carboxylic acid [3-(2-methyl-5-phenyl-oxazole-4-carbonyl)-thiazolidin-2-ylmethyl]-amide;
2,3-Dihydro-benzofuran-4-carboxylic acid [3-(2-methyl-5-phenyl-oxazole-4-carbonyl)-thiazolidin-2-ylmethyl]-amide;
Imidazo[1,2-a]pyridine-3-carboxylic acid [3-(2-methyl-5-phenyl-oxazole-4-carbonyl)-thiazolidin-2-ylmethyl]-amide;
Benzothiazole-7-carboxylic acid {3-[5-(3-fluoro-phenyl)-2-methyl-oxazole-4-carbonyl]-thiazolidin-2-ylmethyl}-amide;
2,3-Dihydro-benzofuran-4-carboxylic acid {3-[5-(3-fluoro-phenyl)-2-methyl-oxazole-4-carbonyl]-thiazolidin-2-ylmethyl}-amide;
2,3-Dihydro-benzo[1,4]dioxine-5-carboxylic acid {3-[5-(3-fluoro-phenyl)-2-methyl-oxazole-4-carbonyl]-thiazolidin-2-ylmethyl}-amide;
1-Methyl-1H-indazole-3-carboxylic acid {3-[5-(3-fluoro-phenyl)-2-methyl-oxazole-4-carbonyl]-thiazolidin-2-ylmethyl}-amide;
Imidazo[1,2-a]pyridine-3-carboxylic acid {3-[5-(3-fluoro-phenyl)-2-methyl-oxazole-4-carbonyl]-thiazolidin-2-ylmethyl}-amide;
Benzothiazole-7-carboxylic acid {3-[5-(3-chloro-phenyl)-2-methyl-oxazole-4-carbonyl]-thiazolidin-2-ylmethyl}-amide;
1-Methyl-1H-indazole-3-carboxylic acid {3-[5-(3-chloro-phenyl)-2-methyl-oxazole-4-carbonyl]-thiazolidin-2-ylmethyl}-amide;
2,3-Dihydro-benzofuran-4-carboxylic acid {3-[5-(3-chloro-phenyl)-2-methyl-oxazole-4-carbonyl]-thiazolidin-2-ylmethyl}-amide;
2,3-Dihydro-benzo[1,4]dioxine-5-carboxylic acid {3-[5-(3-chloro-phenyl)-2-methyl-oxazole-4-carbonyl]-thiazolidin-2-ylmethyl}-amide;
Imidazo[1,2-a]pyridine-3-carboxylic acid {3-[5-(3-chloro-phenyl)-2-methyl-oxazole-4-carbonyl]-thiazolidin-2-ylmethyl}-amide;
2,3-Dihydro-benzofuran-4-carboxylic acid {3-[5-(3-methoxy-phenyl)-2-methyl-oxazole-4-carbonyl]-thiazolidin-2-ylmethyl}-amide;
Benzothiazole-7-carboxylic acid {3-[5-(3-methoxy-phenyl)-2-methyl-oxazole-4-carbonyl]-thiazolidin-2-ylmethyl}-amide;
2,3-Dihydro-benzo[1,4]dioxine-5-carboxylic acid {3-[5-(3-methoxy-phenyl)-2-methyl-oxazole-4-carbonyl]-thiazolidin-2-ylmethyl}-amide;
1-Methyl-1H-indazole-3-carboxylic acid {3-[5-(3-methoxy-phenyl)-2-methyl-oxazole-4-carbonyl]-thiazolidin-2-ylmethyl}-amide;
Imidazo[1,2-a]pyridine-3-carboxylic acid {3-[5-(3-methoxy-phenyl)-2-methyl-oxazole-4-carbonyl]-thiazolidin-2-ylmethyl}-amide;
2,3-Dihydro-benzofuran-4-carboxylic acid [3-(2-m-tolyl-thiophene-3-carbonyl)-thiazolidin-2-ylmethyl]-amide;
Benzothiazole-7-carboxylic acid [3-(2-m-tolyl-thiophene-3-carbonyl)-thiazolidin-2-ylmethyl]-amide;
2,3-Dihydro-benzo[1,4]dioxine-5-carboxylic acid [3-(2-m-tolyl-thiophene-3-carbonyl)-thiazolidin-2-ylmethyl]-amide;
Benzothiazole-7-carboxylic acid {3-[5-(3-fluoro-phenyl)-thiazole-4-carbonyl]-thiazolidin-2-ylmethyl}-amide;
1-Methyl-1H-indazole-3-carboxylic acid {3-[5-(3-fluoro-phenyl)-thiazole-4-carbonyl]-thiazolidin-2-ylmethyl}-amide;
2,3-Dihydro-benzofuran-4-carboxylic acid {3-[5-(3-fluoro-phenyl)-thiazole-4-carbonyl]-thiazolidin-2-ylmethyl}-amide;
2,3-Dihydro-benzo[1,4]dioxine-5-carboxylic acid {3-[5-(3-fluoro-phenyl)-thiazole-4-carbonyl]-thiazolidin-2-ylmethyl}-amide;
Imidazo[1,2-a]pyridine-3-carboxylic acid {3-[5-(3-fluoro-phenyl)-thiazole-4-carbonyl]-thiazolidin-2-ylmethyl}-amide;
2,3-Dihydro-benzofuran-4-carboxylic acid {3-[2-cyclopropyl-5-(3-fluoro-phenyl)-thiazole-4-carbonyl]-thiazolidin-2-ylmethyl}-amide;
2,3-Dihydro-benzo[1,4]dioxine-5-carboxylic acid {3-[2-cyclopropyl-5-(3-fluoro-phenyl)-thiazole-4-carbonyl]-thiazolidin-2-ylmethyl}-amide;
2,3-Dihydro-benzo[1,4]dioxine-5-carboxylic acid {3-[5-(3-fluoro-4-methyl-phenyl)-2-methyl-thiazole-4-carbonyl]-thiazolidin-2-ylmethyl}-amide;
2,3-Dihydro-benzofuran-4-carboxylic acid {3-[5-(3-fluoro-4-methyl-phenyl)-2-methyl-thiazole-4-carbonyl]-thiazolidin-2-ylmethyl}-amide;
Benzothiazole-7-carboxylic acid {3-[5-(3-fluoro-4-methyl-phenyl)-2-methyl-thiazole-4-carbonyl]-thiazolidin-2-ylmethyl}-amide;
2,3-Dihydro-benzofuran-4-carboxylic acid {3-[5-(3,4-difluoro-phenyl)-2-methyl-thiazole-4-carbonyl]-thiazolidin-2-ylmethyl}-amide;
2,3-Dihydro-benzo[1,4]dioxine-5-carboxylic acid {3-[5-(3,4-difluoro-phenyl)-2-methyl-thiazole-4-carbonyl]-thiazolidin-2-ylmethyl}-amide;
Benzothiazole-7-carboxylic acid {3-[5-(2,3-difluoro-4-methyl-phenyl)-2-methyl-thiazole-4-carbonyl]-thiazolidin-2-ylmethyl}-amide;
Imidazo[1,2-a]pyridine-3-carboxylic acid {3-[5-(2,3-difluoro-4-methyl-phenyl)-2-methyl-thiazole-4-carbonyl]-thiazolidin-2-ylmethyl}-amide;
2,3-Dihydro-benzofuran-4-carboxylic acid {3-[5-(2,3-difluoro-4-methyl-phenyl)-2-methyl-thiazole-4-carbonyl]-thiazolidin-2-ylmethyl}-amide;
1-Methyl-1H-indazole-3-carboxylic acid {3-[5-(2,3-difluoro-4-methyl-phenyl)-2-methyl-thiazole-4-carbonyl]-thiazolidin-2-ylmethyl}-amide;
2,3-Dihydro-benzo[1,4]dioxine-5-carboxylic acid {3-[5-(2,3-difluoro-4-methyl-phenyl)-2-methyl-thiazole-4-carbonyl]-thiazolidin-2-ylmethyl}-amide;
2,3-Dihydro-benzofuran-4-carboxylic acid [3-(2-dimethylamino-5-m-tolyl-thiazole-4-carbonyl)-thiazolidin-2-ylmethyl]-amide;
Imidazo[1,2-a]pyridine-3-carboxylic acid [3-(3′,4′-dimethyl-biphenyl-2-carbonyl)-thiazolidin-2-ylmethyl]-amide;
1H-Indazole-7-carboxylic acid [3-(2-methyl-5-m-tolyl-thiazole-4-carbonyl)-thiazolidin-2-ylmethyl]-amide;
6-Fluoro-4H-benzo[1,3]dioxine-8-carboxylic acid [3-(2-methyl-5-m-tolyl-thiazole-4-carbonyl)-thiazolidin-2-ylmethyl]-amide;
1-Methyl-1H-indole-4-carboxylic acid [3-(2-methyl-5-m-tolyl-thiazole-4-carbonyl)-thiazolidin-2-ylmethyl]-amide;
1-Methyl-1H-indole-7-carboxylic acid [3-(2-methyl-5-m-tolyl-thiazole-4-carbonyl)-thiazolidin-2-ylmethyl]-amide;
2,3-Dihydro-benzofuran-7-carboxylic acid [3-(2-methyl-5-m-tolyl-thiazole-4-carbonyl)-thiazolidin-2-ylmethyl]-amide;
1-Methyl-1H-indole-3-carboxylic acid [3-(2-methyl-5-m-tolyl-thiazole-4-carbonyl)-thiazolidin-2-ylmethyl]-amide;
5-Chloro-1,3-dimethyl-1H-pyrazole-4-carboxylic acid [3-(2-methyl-5-m-tolyl-thiazole-4-carbonyl)-thiazolidin-2-ylmethyl]-amide;
3-Ethyl-5-methyl-isoxazole-4-carboxylic acid [3-(2-methyl-5-m-tolyl-thiazole-4-carbonyl)-thiazolidin-2-ylmethyl]-amide;
1-Ethyl-3-methyl-1H-pyrazole-4-carboxylic acid [3-(2-methyl-5-m-tolyl-thiazole-4-carbonyl)-thiazolidin-2-ylmethyl]-amide;
1H-Benzoimidazole-4-carboxylic acid [3-(2-methyl-5-m-tolyl-thiazole-4-carbonyl)-thiazolidin-2-ylmethyl]-amide;
5-Ethyl-3-methyl-isoxazole-4-carboxylic acid [3-(2-methyl-5-m-tolyl-thiazole-4-carbonyl)-thiazolidin-2-ylmethyl]-amide;
3,5-Dimethyl-isoxazole-4-carboxylic acid [3-(2-methyl-5-m-tolyl-thiazole-4-carbonyl)-thiazolidin-2-ylmethyl]-amide;
1-Methyl-5-trifluoromethyl-1H-pyrazole-4-carboxylic acid [3-(2-methyl-5-m-tolyl-thiazole-4-carbonyl)-thiazolidin-2-ylmethyl]-amide;
1,3-Dimethyl-1H-pyrazole-4-carboxylic acid [3-(2-methyl-5-m-tolyl-thiazole-4-carbonyl)-thiazolidin-2-ylmethyl]-amide;
Benzo[1,2,3]thiadiazole-5-carboxylic acid [3-(2-methyl-5-m-tolyl-thiazole-4-carbonyl)-thiazolidin-2-ylmethyl]-amide;
Benzothiazole-6-carboxylic acid [3-(2-methyl-5-m-tolyl-thiazole-4-carbonyl)-thiazolidin-2-ylmethyl]-amide;
1,3,5-Trimethyl-1H-pyrazole-4-carboxylic acid [3-(2-methyl-5-m-tolyl-thiazole-4-carbonyl)-thiazolidin-2-ylmethyl]-amide;
2,2-Dimethyl-2,3-dihydro-benzofuran-7-carboxylic acid [3-(2-methyl-5-m-tolyl-thiazole-4-carbonyl)-thiazolidin-2-ylmethyl]-amide;
2,2-Difluoro-benzo[1,3]dioxole-4-carboxylic acid [3-(2-methyl-5-m-tolyl-thiazole-4-carbonyl)-thiazolidin-2-ylmethyl]-amide;
1-Methyl-1H-indole-5-carboxylic acid [3-(2-methyl-5-m-tolyl-thiazole-4-carbonyl)-thiazolidin-2-ylmethyl]-amide;
2,3-Dihydro-benzo[1,4]dioxine-6-carboxylic acid [3-(2-methyl-5-m-tolyl-thiazole-4-carbonyl)-thiazolidin-2-ylmethyl]-amide;
3H-Benzoimidazole-5-carboxylic acid [3-(2-methyl-5-m-tolyl-thiazole-4-carbonyl)-thiazolidin-2-ylmethyl]-amide;
1-Isopropyl-1H-pyrazole-4-carboxylic acid [3-(2-methyl-5-m-tolyl-thiazole-4-carbonyl)-thiazolidin-2-ylmethyl]-amide;
2-Methyl-benzothiazole-5-carboxylic acid [3-(2-methyl-5-m-tolyl-thiazole-4-carbonyl)-thiazolidin-2-ylmethyl]-amide;
1-Methyl-1H-benzotriazole-5-carboxylic acid [3-(2-methyl-5-m-tolyl-thiazole-4-carbonyl)-thiazolidin-2-ylmethyl]-amide;
1-Methyl-1H-benzoimidazole-5-carboxylic acid [3-(2-methyl-5-m-tolyl-thiazole-4-carbonyl)-thiazolidin-2-ylmethyl]-amide;
Quinoxaline-5-carboxylic acid [3-(2-methyl-5-m-tolyl-thiazole-4-carbonyl)-thiazolidin-2-ylmethyl]-amide;
1H-Indazole-4-carboxylic acid [3-(2-methyl-5-m-tolyl-thiazole-4-carbonyl)-thiazolidin-2-ylmethyl]-amide;
4-Chloro-pyridine-2-carboxylic acid [3-(2-methyl-5-m-tolyl-thiazole-4-carbonyl)-thiazolidin-2-ylmethyl]-amide;
4-Methoxy-pyridine-2-carboxylic acid [3-(2-methyl-5-m-tolyl-thiazole-4-carbonyl)-thiazolidin-2-ylmethyl]-amide;
2-Methoxy-N-[3-(2-methyl-5-m-tolyl-thiazole-4-carbonyl)-thiazolidin-2-ylmethyl]-nicotinamide;
6-Methyl-pyridine-2-carboxylic acid [3-(2-methyl-5-m-tolyl-thiazole-4-carbonyl)-thiazolidin-2-ylmethyl]-amide;
6-Trifluoromethyl-pyridine-2-carboxylic acid [3-(2-methyl-5-m-tolyl-thiazole-4-carbonyl)-thiazolidin-2-ylmethyl]-amide;
2,5-Dimethyl-oxazole-4-carboxyl ic acid [3-(2-methyl-5-m-tolyl-thiazole-4-carbonyl)-thiazolidin-2-ylmethyl]-amide;
6-Methoxy-pyridine-2-carboxylic acid [3-(2-methyl-5-m-tolyl-thiazole-4-carbonyl)-thiazolidin-2-ylmethyl]-amide;
2,4-Dimethyl-thiazole-5-carboxylic acid [3-(2-methyl-5-m-tolyl-thiazole-4-carbonyl)-thiazolidin-2-ylmethyl]-amide;
2-Methyl-thiazole-4-carboxylic acid [3-(2-methyl-5-m-tolyl-thiazole-4-carbonyl)-thiazolidin-2-ylmethyl]-amide;
4-Methyl-thiazole-5-carboxylic acid [3-(2-methyl-5-m-tolyl-thiazole-4-carbonyl)-thiazolidin-2-ylmethyl]-amide;
3-Methyl-quinoxaline-2-carboxylic acid [3-(2-methyl-5-m-tolyl-thiazole-4-carbonyl)-thiazolidin-2-ylmethyl]-amide;
5-Chloro-pyridine-2-carboxyl ic acid [3-(2-methyl-5-m-tolyl-thiazole-4-carbonyl)-thiazolidin-2-ylmethyl]-amide;
5-Methyl-thiazole-2-carboxylic acid [3-(2-methyl-5-m-tolyl-thiazole-4-carbonyl)-thiazolidin-2-ylmethyl]-amide;
2,6-Dimethoxy-N-[3-(2-methyl-5-m-tolyl-thiazole-4-carbonyl)-thiazolidin-2-ylmethyl]-nicotinamide;
Thiazole-4-carboxylic acid [3-(2-methyl-5-m-tolyl-thiazole-4-carbonyl)-thiazolidin-2-ylmethyl]-amide;
2,4-Dimethyl-oxazole-5-carboxylic acid [3-(2-methyl-5-m-tolyl-thiazole-4-carbonyl)-thiazolidin-2-ylmethyl]-amide;
4-Methyl-thiazole-2-carboxylic acid [3-(2-methyl-5-m-tolyl-thiazole-4-carbonyl)-thiazolidin-2-ylmethyl]-amide;
6-Methyl-N-[3-(2-methyl-5-m-tolyl-thiazole-4-carbonyl)-thiazolidin-2-ylmethyl]-nicotinamide;
5-Methyl-N-[3-(2-methyl-5-m-tolyl-thiazole-4-carbonyl)-thiazolidin-2-ylmethyl]-nicotinamide;
4-Methyl-oxazole-5-carboxylic acid [3-(2-methyl-5-m-tolyl-thiazole-4-carbonyl)-thiazolidin-2-ylmethyl]-amide;
[2,6]Naphthyridine-3-carboxylic acid [3-(2-methyl-5-m-tolyl-thiazole-4-carbonyl)-thiazolidin-2-ylmethyl]-amide;
[1,5]Naphthyridine-2-carboxylic acid [3-(2-methyl-5-m-tolyl-thiazole-4-carbonyl)-thiazolidin-2-ylmethyl]-amide;
Quinoxaline-2-carboxylic acid [3-(2-methyl-5-m-tolyl-thiazole-4-carbonyl)-thiazolidin-2-ylmethyl]-amide;
5-Methyl-isoxazole-3-carboxylic acid [3-(2-methyl-5-m-tolyl-thiazole-4-carbonyl)-thiazolidin-2-ylmethyl]-amide;
[1,8]Naphthyridine-2-carboxylic acid [3-(2-methyl-5-m-tolyl-thiazole-4-carbonyl)-thiazolidin-2-ylmethyl]-amide;
4-Methyl-N-[3-(2-methyl-5-m-tolyl-thiazole-4-carbonyl)-thiazolidin-2-ylmethyl]-nicotinamide;
N-[3-(2-Methyl-5-m-tolyl-thiazole-4-carbonyl)-thiazolidin-2-ylmethyl]-6-trifluoromethyl-nicotinamide;
1H-Pyrazolo[3,4-b]pyridine-5-carboxylic acid [3-(2-methyl-5-m-tolyl-thiazole-4-carbonyl)-thiazolidin-2-ylmethyl]-amide;
Oxazole-4-carboxylic acid [3-(2-methyl-5-m-tolyl-thiazole-4-carbonyl)-thiazolidin-2-ylmethyl]-amide;
N-[3-(2-Methyl-5-m-tolyl-thiazole-4-carbonyl)-thiazolidin-2-ylmethyl]-4-trifluoromethyl-nicotinamide;
1H-Pyrazolo[3,2-b]pyridine-6-carboxylic acid [3-(2-methyl-5-m-tolyl-thiazole-4-carbonyl)-thiazolidin-2-ylmethyl]-amide;
4-Chloro-N-[3-(2-methyl-5-m-tolyl-thiazole-4-carbonyl)-thiazolidin-2-ylmethyl]-nicotinamide;
Benzothiazole-7-carboxylic acid {3-[2-dimethylamino-5-(3-methoxy-phenyl)-thiazole-4-carbonyl]-thiazolidin-2-ylmethyl}-amide;
Benzothiazole-7-carboxylic acid {3-[2-dimethylamino-5-(4-fluoro-phenyl)-thiazole-4-carbonyl]-thiazolidin-2-ylmethyl}-amide;
Benzothiazole-7-carboxylic acid {3-[2-dimethylamino-5-(3-fluoro-phenyl)-thiazole-4-carbonyl]-thiazolidin-2-ylmethyl}-amide;
Benzothiazole-7-carboxylic acid {3-[2-dimethylamino-5-(3,4-dimethyl-phenyl)-thiazole-4-carbonyl]-thiazolidin-2-ylmethyl}-amide;
Benzothiazole-7-carboxylic acid [3-(3-m-tolyl-pyrazine-2-carbonyl)-thiazolidin-2-ylmethyl]-amide;
Benzothiazole-7-carboxylic acid {3-[3-(3,4-dimethyl-phenyl)-pyrazine-2-carbonyl]-thiazolidin-2-ylmethyl}-amide; and
Benzothiazole-7-carboxylic acid {3-[3-(3-methoxy-phenyl)-pyrazine-2-carbonyl]-thiazolidin-2-ylmethyl}-amide;
or a salt thereof.

9. A compound according to claim 1 selected from the group consisting of:
2,3-Dihydro-benzofuran-4-carboxylic acid {3-[5-(3-chloro-phenyl)-thiazole-4-carbonyl]-thiazolidin-2-ylmethyl}-amide;
2,3-Dihydro-benzofuran-4-carboxylic acid {3-[5-(3,4-dimethyl-phenyl)-thiazole-4-carbonyl]-thiazolidin-2-ylmethyl}-amide;
2,3-Dihydro-benzofuran-4-carboxylic acid {3-[2-dimethylamino-5-(4-fluoro-phenyl)-thiazole-4-carbonyl]-thiazolidin-2-ylmethyl}-amide;
2,3-Dihydro-benzofuran-4-carboxylic acid {3-[2-dimethylamino-5-(2-fluoro-phenyl)-thiazole-4-carbonyl]-thiazolidin-2-ylmethyl}-amide;
2,3-Dihydro-benzofuran-4-carboxylic acid {3-[2-(ethyl-methyl-amino)-5-(3-methoxy-phenyl)-thiazole-4-carbonyl]-thiazolidin-2-ylmethyl}-amide;
2,3-Dihydro-benzofuran-4-carboxylic acid {3-[2-(ethyl-methyl-amino)-5-(4-fluoro-phenyl)-thiazole-4-carbonyl]-thiazolidin-2-ylmethyl}-amide;
2,3-Dihydro-benzofuran-4-carboxylic acid {3-[2-dimethylamino-5-(3-methoxy-phenyl)-thiazole-4-carbonyl]-thiazolidin-2-ylmethyl}-amide;
2,3-Dihydro-benzofuran-4-carboxylic acid {3-[5-(3-methoxy-phenyl)-thiazole-4-carbonyl]-thiazolidin-2-ylmethyl}-amide;
2,3-Dihydro-benzofuran-4-carboxylic acid [3-(5-m-tolyl-thiazole-4-carbonyl)-thiazolidin-2-ylmethyl]-amide;
2,3-Dihydro-benzofuran-4-carboxylic acid [3-(2-dimethylamino-5-p-tolyl-thiazole-4-carbonyl)-thiazolidin-2-ylmethyl]-amide;
2,3-Dihydro-benzofuran-4-carboxylic acid {3-[2-dimethylamino-5-(3-fluoro-4-methyl-phenyl)-thiazole-4-carbonyl]-thiazolidin-2-ylmethyl}-amide;
2,3-Dihydro-benzofuran-4-carboxylic acid {3-[5-(3-chloro-phenyl)-2-dimethylamino-thiazole-4-carbonyl]-thiazolidin-2-ylmethyl}-amide;
2,3-Dihydro-benzofuran-4-carboxylic acid {3-[3-(3,4-dimethyl-phenyl)-pyrazine-2-carbonyl]-thiazolidin-2-ylmethyl}-amide;
2,3-Dihydro-benzofuran-4-carboxylic acid {3-[3-(4-fluoro-3-methyl-phenyl)-pyrazine-2-carbonyl]-thiazolidin-2-ylmethyl}-amide;
Benzothiazole-7-carboxylic acid {3-[2-dimethylamino-5-(4-fluoro-phenyl)-thiazole-4-carbonyl]-thiazolidin-2-ylmethyl}-amide;
Benzothiazole-7-carboxylic acid {3-[2-dimethylamino-5-(3-methoxy-phenyl)-thiazole-4-carbonyl]-thiazolidin-2-ylmethyl}-amide;
Benzothiazole-7-carboxylic acid [3-(2-dimethylamino-5-p-tolyl-thiazole-4-carbonyl)-thiazolidin-2-ylmethyl]-amide;
Benzothiazole-7-carboxylic acid {3-[2-dimethylamino-5-(3-fluoro-4-methyl-phenyl)-thiazole-4-carbonyl]-thiazolidin-2-ylmethyl}-amide;
1-Methyl-1H-indole-3-carboxylic acid {3-[5-(4-fluoro-phenyl)-thiazole-4-carbonyl]-thiazolidin-2-ylmethyl}-amide;
1-Methyl-5-trifluoromethyl-1H-pyrazole-4-carboxylic acid {3-[5-(4-fluoro-phenyl)-thiazole-4-carbonyl]-thiazolidin-2-ylmethyl}-amide;
1-Methyl-1H-indole-3-carboxylic acid {3-[5-(3-fluoro-phenyl)-thiazole-4-carbonyl]thiazolidin-2-ylmethyl}-amide;
1-Methyl-5-trifluoromethyl-1H-pyrazole-4-carboxylic acid {3-[5-(3-fluoro-phenyl)-thiazole-4-carbonyl]-thiazolidin-2-ylmethyl}-amide;
1-Ethyl-3-methyl-1H-pyrazole-4-carboxylic acid {3-[5-(3-fluoro-phenyl)-thiazole-4-carbonyl]-thiazolidin-2-ylmethyl}-amide;
1,3-Dimethyl-1H-pyrazole-4-carboxylic acid {3-[5-(3-fluoro-phenyl)-thiazole-4-carbonyl]-thiazolidin-2-ylmethyl}-amide; and
Quinoxaline-5-carboxylic acid {3-[5-(3-fluoro-phenyl)-thiazole-4-carbonyl]-thiazolidin-2-ylmethyl}-amide;
or a salt thereof.

10. A pharmaceutical composition containing, as active principle, a compound according to claim 1, or a pharmaceutically acceptable salt thereof, and at least one therapeutically inert excipient.

11. A pharmaceutical composition according to claim 10, wherein the compound is in the form of a pharmaceutically acceptable salt thereof.

12. A method for the prevention or treatment of diseases selected from the group consisting of all types of sleep disorders, of stress-related syndromes, of psychoactive substance use, abuse, seeking and reinstatement, of cognitive dysfunctions in the healthy population and in psychiatric and neurologic disorders, of eating or drinking disorders comprising administering a compound according to claim 1, or a pharmaceutically acceptable salt thereof, to a patient in need thereof.

13. (canceled)
